(19) United States
(12) Patent Application Publication
(10) Pub. No.: US 2025/0295632 A1
(43) Pub. Date: Sep. 25, 2025

(54) COMPOUNDS AND METHODS FOR TREATING FRIEDREICH'S ATAXIA

(71) Applicant: Design Therapeutics, Inc., Carlsbad, CA (US)

(72) Inventors: Chengzhi ZHANG, Carlsbad, CA (US); Abhijit BHAT, Carlsbad, CA (US)

(21) Appl. No.: 18/863,942

(22) PCT Filed: May 8, 2023
(86) PCT No.: PCT/US2023/021335

§ 371 Date: Nov. 7, 2024

Related U.S. Application Data
(60) Provisional application No. 63/339,708, filed on May 9, 2022.

(51) Int. Cl. A61K 31/4164; A61P 25/14

(52) U.S. Cl. CPC A61K 31/4164; A61P 25/14

(57) ABSTRACT

The present disclosure relates to compounds and methods for modulating the expression of fxn, and treating diseases and conditions in which fxn plays an active role.

### CROSS REFERENCE TO RELATED APPLICATIONS

This application claims the benefit of U.S. Application No. 63/339,708, filed May 9, 2022, which is hereby incorporated by reference in its entirety.

### FIELD OF THE DISCLOSURE

Disclosed herein are new chimeric heterocyclic polyamide compounds and compositions and their application as pharmaceuticals for the treatment of disease. Methods to modulate the expression of fxn in a human or animal subject are also provided for the treatment diseases such as Friedreich's ataxia.

### BACKGROUND OF THE DISCLOSURE

The disclosure relates to the treatment of inherited genetic diseases characterized by overproduction of mRNA.

Friedreich's ataxia (“FA” or “FRDA”) is an autosomal recessive neurodegenerative disorder caused by mutations in the fxn gene, which encodes the protein frataxin (“FXN”), an iron-binding mitochondrial protein involved in electron transport and metabolism. In most subjects with FA, a GAA trinucleotide repeat (from about 66 to over 1000 trinucleotides) is included in the first intron of fxn, and this hyperexpansion is responsible for the observed pathology. Hyperexpansion of the GAA repeats results in reduced expression of FXN.

Friedreich's ataxia is characterized by progressive degradation of the nervous system, particularly sensory neurons. In addition, cardiomyocytes and pancreatic beta cells are susceptible to frataxin depletion. Symptoms usually present by age 18; however, later diagnoses of FA are not uncommon. FA patients develop neurodegeneration of the large sensory neurons and spinocerebellar tracts, as well as cardiomyopathy and diabetes mellitus. Clinical symptoms of FA include ataxia, gait ataxia, muscle weakness, loss of upper body strength, loss of balance, lack of reflexes in lower limbs and tendons, loss of sensation, particularly to vibrations, impairment of position sense, impaired perception of temperature, touch, and pain, hearing and vision impairment, including distorted color vision and involuntary eye movements, irregular foot configuration, including pes cavus and inversion, hearing impairment, dysarthria, dysphagia, impaired breathing, scoliosis, diabetes, intolerance to glucose and carbohydrates, cardiac dysfunctions including hypertrophic cardiomyopathy, arrhythmia, myocardial fibrosis, and cardiac failure. Currently there is no cure for FA, with medical treatments being limited to surgical intervention for the spine and the heart, as well as therapy to assist with balance, coordination, motion, and speech.

### SUMMARY OF THE DISCLOSURE

This disclosure utilizes regulatory molecules present in cell nuclei that control gene expression. Eukaryotic cells provide several mechanisms for controlling gene replication, transcription, and/or translation. Regulatory molecules that are produced by various biochemical mechanisms within the cell can modulate the various processes involved in the conversion of genetic information to cellular components. Several regulatory molecules are known to modulate the production of mRNA and, if directed to fxn, could modulate the production of fxn mRNA that causes Friedreich's ataxia, and thus, reverse the progress of the disease.

The disclosure provides compounds and methods for recruiting a regulatory molecule into close proximity to fxn. The compounds disclosed herein contain: (a) a recruiting moiety that will bind to a regulatory molecule, linked to (b) a DNA binding moiety that will selectively bind to fxn. The compounds will counteract the expression of defective fxn in the following manner:
(1) The DNA binding moiety will bind selectively the characteristic GAA trinucleotide repeat sequence of fxn;
(2) The recruiting moiety, linked to the DNA binding moiety, will thus be held in proximity to fxn; (3) The recruiting moiety, now in proximity to fxn, will recruit the regulatory molecule into proximity with the gene; and
(4) The regulatory molecule will modulate expression, and therefore counteract the expression of defective fxn by direct interaction with the gene.

The mechanism set forth above will provide an effective treatment for Friedreich's ataxia, which is caused by the expression of defective fxn gene. Correction of the expression of the defective fxn gene thus represents a promising method for the treatment of Friedreich's ataxia.

The disclosure provides recruiting moieties that will bind to regulatory molecules. Small molecule inhibitors of regulatory molecules serve as templates for the design of recruiting moieties, since these inhibitors generally act via noncovalent binding to the regulatory molecules.

The disclosure further provides for DNA binding moieties that will selectively bind to one or more copies of the GAA trinucleotide repeat that is characteristic of the defective fxn gene. Selective binding of the DNA binding moiety to fxn, made possible due to the high GAA count associated with the defective fxn gene, will direct the recruiting moiety into proximity of the gene, and recruit the regulatory molecule into position to up-regulate gene transcription.

The DNA binding moiety will comprise a polyamide segment that will bind selectively to the target GAA sequence. Polyamides have been designed by Dervan (U.S. Pat. Nos. 9,630,950 and 8,524,899) and others that can selectively bind to selected DNA sequences. These polyamides sit in the minor groove of double helical DNA and form hydrogen bonding interactions with the Watson-Crick base pairs. Polyamides that selectively bind to particular DNA sequences can be designed by linking monoamide building blocks according to established chemical rules. One building block is provided for each DNA base pair, with each building block binding noncovalently and selectively to one of the DNA base pairs: A/T, T/A, G/C, and C/G. Following this guideline, trinucleotides will bind to molecules with three amide units, i.e. triamides. In general, these polyamides will orient in either direction of a DNA sequence, so that the 5′-GAA-3′ trinucleotide repeat sequence of fxn can be targeted by the polyamides selective either for GAA or for AAG. Furthermore, polyamides that bind to the complementary sequence, in this case, TTC or CTT, will also bind to the trinucleotide repeat sequence of fxn and can be employed as well.

In principle, longer DNA sequences can be targeted with higher specificity and/or higher affinity by combining a larger number of monoamide building blocks into longer polyamide chains. Ideally, the binding affinity for a polyamide would simply be equal to the sum of each individual monoamide/DNA base pair interaction. In practice, however, due to the geometric mismatch between the fairly rigid polyamide and DNA structures, longer polyamide sequences do not bind to longer DNA sequences as tightly as would be expected from a simple additive contribution. The geometric mismatch between longer polyamide sequences and longer DNA sequences induces an unfavorable geometric strain that subtracts from the binding affinity that would be otherwise expected.

The disclosure, therefore, provides DNA moieties that comprise triamides that are connected by flexible spacers. The spacers alleviate the geometric strain that would otherwise decrease binding affinity of a larger polyamide sequence.

Disclosed herein are compounds that comprise a polyamide which can bind to one or more copies of the trinucleotide repeat sequence GAA, and can modulate the expression of the defective fxn gene. Treatment of a subject with these compounds may counteract the expression of the defective fxn gene, and this can reduce the occurrence, severity, and/or frequency of symptoms associated with Friedreich's ataxia. Certain compounds disclosed herein may provide higher binding affinity and/or selectivity than has been observed previously for this class of compound.

In another aspect disclosed herein is a pharmaceutical composition comprising a compound disclosed herein or a pharmaceutically acceptable salt thereof; and a pharmaceutically acceptable excipient.

In another aspect disclosed herein a method of modulation of the expression of fxn comprising contacting fxn with a compound disclosed, or a pharmaceutically acceptable salt thereof.

In another aspect disclosed herein is a method of treating a disease or condition caused by expression of a defective fxn in a patient in need thereof, comprising administering to the patient therapeutically effective amount of a compound of disclosed herein, or a pharmaceutically acceptable salt thereof. In some embodiments, the disease is Friedreich's ataxia (FA).

Other objects, features, and advantages of the compounds, methods, and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE DISCLOSURE

The disclosed herein are compounds (i.e., transcription modulator molecules) that contain DNA binding moieties that can selectively bind to one or more copies of the GAA trinucleotide repeat that is characteristic of the defective fxn gene. The compounds also contains moieties that bind to regulatory proteins. The selective binding of the target gene can bring the regulatory protein into proximity to the target gene and thus downregulates transcription of the target gene. The compounds disclosed herein provide higher binding affinity and selectivity than has been observed previously for this class of compounds and can be more effective in treating diseases associated with the defective fxn gene.

The compounds described herein can recruit the regulatory molecule to modulate the expression of the defective fxn gene and effectively treat and/or and alleviate the symptoms associated with diseases such as Friedreich's ataxia.

### Compounds

The compounds disclosed herein possess useful activity for modulating the transcription of a target gene having one or more GAA repeats (e.g., fxn), and may be used in the treatment or prophylaxis of a disease or condition in which the target gene (e.g., fxn) plays an active role. Thus, some embodiments also provide for pharmaceutical compositions comprising one or more compounds disclosed herein together with a pharmaceutically acceptable carrier, as well as methods of making and using the compounds and compositions. Some embodiments provide methods for modulating the expression of fxn. Other embodiments provide methods for treating a fxn-mediated disorder in a patient in need of such treatment, comprising administering to the patient a therapeutically effective amount of a compound or composition according to the present disclosure. Also provided herein are methods of treating a disease or condition that would be ameliorated by the modulation of the expression of fxn.

In an aspect, provided herein is a transcription modulator molecule having a first terminus, a second terminus, and a linker moiety, wherein:
(a) the first terminus comprises a DNA-binding moiety capable of binding a nucleotide repeat comprising GAA;
(b) the second terminus comprises a protein-binding moiety capable of binding to a regulatory molecule that modulates expression of a gene having the expanded GAA repeat; and
(c) the linker comprising an oligomeric backbone that connects the first terminus and the second terminus.

In some embodiments, the DNA-binding moiety is a polyamide.

In some embodiments, the second terminus is a bromodomain binding moiety. In some embodiments, the bromodomain binding moiety is a BET binding moiety that is not BRD4. In some embodiments, the bromodomain binding moiety is a non-BET binding moiety.

### First Terminus—DNA Binding Moiety

The first terminus interacts and binds with the gene, particularly with the minor grooves of the GAA sequence. In one aspect, the compounds of the present disclosure provide a polyamide sequence for interaction of a single polyamide subunit to each base pair in the GAA repeat sequence. In one aspect, the compounds of the present disclosure provide a turn component (e.g., aliphatic amino acid moiety), in order to enable hairpin binding of the compound to the GAA, in which each nucleotide pair interacts with two subunits of the polyamide.

In an aspect, the compounds disclosed herein are more likely to bind to the repeated GAA of fxn than to GAA elsewhere in the subject's DNA, due to the high number of GAA repeats associated with fxn.

In some embodiments, the compounds disclosed herein provide more than one copy of the polyamide sequence for noncovalent binding to GAA. In some embodiments, the compounds of the present disclosure bind to fxn with an affinity that is greater than a corresponding compound that contains a single polyamide sequence.

In some embodiments, the DNA recognition or binding moiety binds in the minor groove of DNA.

In some embodiments, the DNA recognition or binding moiety comprises a polymeric sequence of monomers, wherein each monomer in the polymer selectively binds to a certain DNA base pair.

In some embodiments, the DNA recognition or binding moiety comprises a polyamide moiety.

In some embodiments, the DNA recognition or binding moiety comprises a polyamide moiety comprising heteroaromatic monomers, wherein each heteroaromatic monomer binds noncovalently to a specific nucleotide, and each heteroaromatic monomer is attached to its neighbor or neighbors via amide bonds.

In some embodiments, the DNA recognition moiety binds to a sequence comprising at least 1000 trinucleotide repeats. In some embodiments, the DNA recognition moiety binds to a sequence comprising at least 500 trinucleotide repeats. In some embodiments, the DNA recognition moiety binds to a sequence comprising at least 200 trinucleotide repeats. In some embodiments, the DNA recognition moiety binds to a sequence comprising at least 100 trinucleotide repeats. In some embodiments, the DNA recognition moiety binds to a sequence comprising at least 50 trinucleotide repeats. In some embodiments, the DNA recognition moiety binds to a sequence comprising at least 20 trinucleotide repeats.

The form of the polyamide selected can vary based on the target gene. The first terminus can include a polyamide selected from the group consisting of a linear polyamide, a hairpin polyamide, a H-pin polyamide, an U-pin polyamide, an overlapped polyamide, a slipped polyamide, a cyclic polyamide, a tandem polyamide, and an extended polyamide. In some embodiments, the first terminus comprises a linear polyamide. In some embodiments, the first terminus comprises a hairpin polyamide.

The binding affinity between the polyamide and the target gene can be adjusted based on the composition of the polyamide. In some embodiments, the polyamide is capable of binding the DNA with an affinity of less than about 600 nM, about 500 nM, about 400 nM, about 300 nM, about 250 nM, about 200 nM, about 150 nM, about 100 nM, or about 50 nM. In some embodiments, the polyamide is capable of binding the DNA with an affinity of less than about 300 nM. In some embodiments, the polyamide is capable of binding the DNA with an affinity of less than about 200 nM.

The binding affinity between the polyamide and the target DNA can be determined using a quantitative footprint titration experiment. The experiment involve measuring the dissociation constant K_{d} of the polyamide for target sequence at either 24° C. or 37° C., and using either standard polyamide assay solution conditions or approximate intracellular solution conditions.

The binding affinity between the regulatory protein and the ligand on the second terminus can be determined using an assay suitable for the specific protein. The experiment involve measuring the dissociation constant K_{d} of the ligand for protein and using either standard protein assay solution conditions or approximate intracellular solution conditions.

In some embodiments, the DNA-binding moiety comprises a polyamide of one or more of the following subunits selected from

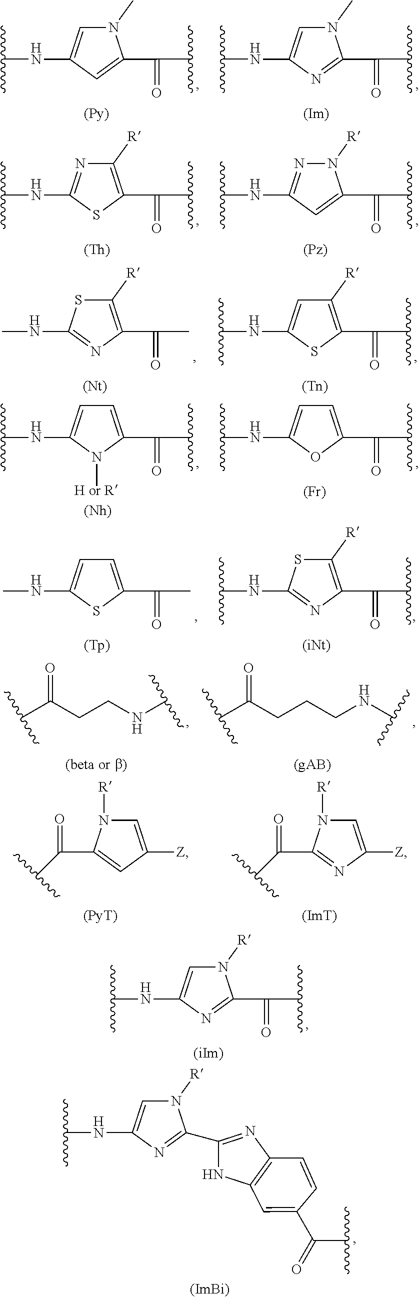

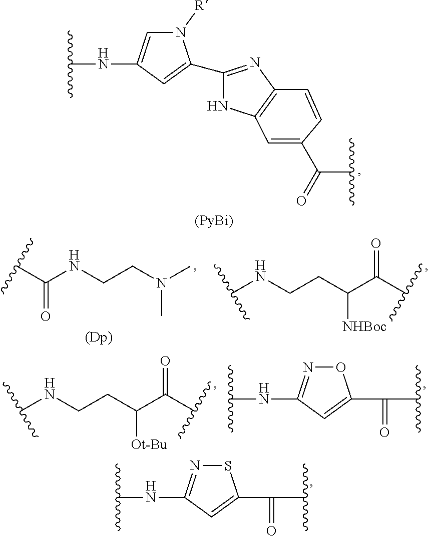

—NH-benzopyrazinylene-C(O)—, —NH-phenylene-C(O)—, —NH-pyridinylene-C(O)—, —NH-piperidinylene-C(O)—, —NH-pyrirnidinylene-C(O)—, —NH-anthracenylene-C(O)—, —NH-quinolinylene-C(O)—, and

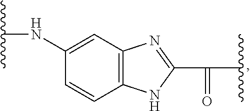

wherein each R′ is independently hydrogen, optionally substituted C₁-C₂₀ alkyl, C₁-C₂₀ heteroalkyl, C₁-C₂₀ haloalkyl, or C₁-C₂₀ alkylamino; and Z is H, NH₂, C₁₋₆ alkyl, C₁-C₆ haloalkyl or C₁-C₆ alkyl-NH₂.

In some embodiments, the monomer element is independently selected from the group consisting of optionally substituted pyrrole carboxamide monomer, optionally substituted imidazole carboxamide monomer, optionally substituted C—C linked heteromonocyclic/heterobicyclic moiety, and D-alanine. In some embodiments, one or more of the polyamide backbone carbonyl groups (C═O), is replaced with an oxetane. In some embodiments, at least one of the polyamide backbone carbonyl groups is replaced with an oxetane.

In some embodiments, the first terminus comprises one or more subunits selected from the group consisting of optionally substituted N-methylpyrrole, optionally substituted N-methylimidazole, and (3-alanine.

In some embodiments, the first terminus comprises a polyamide having the structure of Formula (A), or a pharmaceutically acceptable salt thereof:

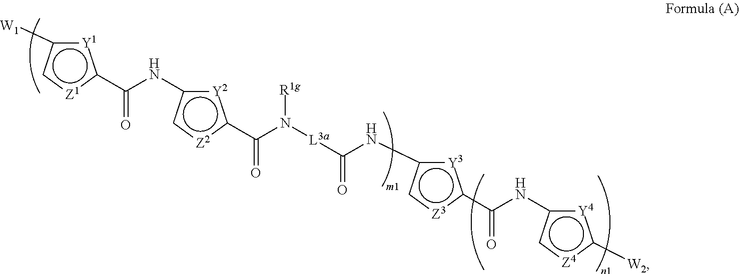

wherein;
m₁ is 1-4;
n₁ is 0-2;
each Y¹, Y², Y³, and Y⁴ is independently CH or N;
each Z¹, Z², Z³, and Z⁴ is independently O, S. or NR²;
W₁ is hydrogen, deuterium, halogen, optionally substituted C₁-C₁₀ alkyl, —NR^{1e}C(O)NR^{1e}R^{1f}, —C(O)NR^{1e}R^{1f}, —O—C(O)NR^{1e}R^{1f}, —NR^{1e}C(O)—OR^{1f}, or (AA)₁₋₁₀;
W₂ is hydrogen, deuterium, halogen, optionally substituted C₁-C₁₀ alkyl, —C(O)NR^{1e}R^{1f}, or (AA)₁₋₁₀; wherein
   each R^{1e} is independently hydrogen, optionally substituted C₁-C₅₀ alkyl, optionally substituted C₁-C₅₀ alkenyl, optionally substituted C₁-C₅₀ alkynyl, optionally substituted C₁-C₅₀ heteroalkyl, optionally substituted C₁-C₅₀ heteroalkenyl, optionally substituted C₁-C₅₀ heteroalkynyl, or PEG₁₋₅₀;
   each R^{1f} is independently hydrogen, optionally substituted C₁-C₂₀ alkyl, optionally substituted C₁-C₂₀ alkenyl, optionally substituted C₁-C₂₀ alkynyl, optionally substituted C₁-C₂₀ heteroalkyl, optionally substituted C₁-C₂₀ heteroalkenyl, optionally substituted C₁-C₂₀ heteroalkynyl, PEG₁₋₂₀, or one or more AA;
   or R^{1e} and R^{1f} together with the nitrogen atom to which they are attached form an optionally substituted heterocycloalkyl;
   each AA is independently a naturally occurring amino acid;
each R² is independently hydrogen, optionally substituted C₁-C₅₀ alkyl, optionally substituted C₁-C₅₀ alkenyl, optionally substituted C₁-C₅₀ alkynyl, optionally substituted C₁-C₅₀ heteroalkyl, optionally substituted C₁-C₅₀ heteroalkyl, optionally substituted C₁-C₅₀ heteroalkenyl, optionally substituted C₁-C₅₀ heteroalkynyl, optionally substituted C₃-C₈ cycloalkyl, or optionally substituted 3 to 8-membered heterocycloalkyl, or PEG₁₋₅₀;
each L^{3a} is an optionally substituted C₁-C₆ alkylene, optionally substituted C₃-C₇ cycloalkylene, optionally substituted 3 to 7-membered heterocyclene, or optionally substituted 5 to 6-membered heteroarylene;
each R^{1g} is hydrogen or C₁-C₆ alkyl;
or R^{1g} and L^{3a} together with the atom(s) to which they are attached form an optionally substituted 4 to 8-membered heterocycloalkyl.

In some embodiments, each L^{3a} is an optionally substituted C₁-C₆ alkylene. In some embodiments, L^{3a} is a C₂, C₃, C₄, or C₅ alkylene optionally substituted with one or more hydrogen, halogen, hydroxyl, C₁-C₆ alkyl, C₁-C₆ heteroalkyl, C₁-C₆ haloalkyl, C₁-C₆ hydroxyalkyl, C₃-C₆ cycloalkyl or 4 to 7-membered heterocycloalkyl ring. In some embodiments. L^{3a} is a C₂ or C₃ alkylene optionally substituted with one or more hydrogen, halogen, C₁-C₆ alkyl, C₁-C₆ heteroalkyl, C₃-C₆ cycloalkyl or 4 to 7-membered heterocycloalkyl ring. In some embodiments. L^{3a} is a C₂ alkylene optionally substituted with one or two hydrogen, C₁-C₆ alkyl, C₁-C₆ heteroalkyl, C₃-C₆ cycloalkyl or 4 to 7-membered heterocycloalkyl ring.

In some embodiments, each L^{3a} is independently C₃-C₇ cycloalkylene. In some embodiments, L^{3a} is a cyclobutylene, cyclopentylene, cyclohexylene, or cycloheptylene ring. In some embodiments, L³ is cyclobutylene. In some embodiments, L^{3a} is cyclopentylene. In some embodiments, L^{3a} is cyclohexylene.

In some embodiments, each L^{3a} is 3 to 7-membered heterocyclene. In some embodiments, L^{3a} is a 4-membered, 5-membered, or 6-membered heterocyclene.

In some embodiments, each R^{1g} is independently hydrogen. In some embodiments, each R^{1g} is independently C₁-C₆ alkyl.

In some embodiments, L^{3a} and R^{1g} together with the atoms to which they are attached form a 4 to 7-membered heterocycloalkyl. In some embodiments, the ring is a 4-membered heterocycloalkyl. In some embodiments, the ring is a 5-membered heterocycloalkyl. In some embodiments, the ring is a 6-membered heterocycloalkyl. In some embodiments, the ring is a 7-membered heterocycloalkyl.

In some embodiments, the first terminus comprises a polyamide having the structure of Formula (A-1), or a pharmaceutically acceptable salt thereof:

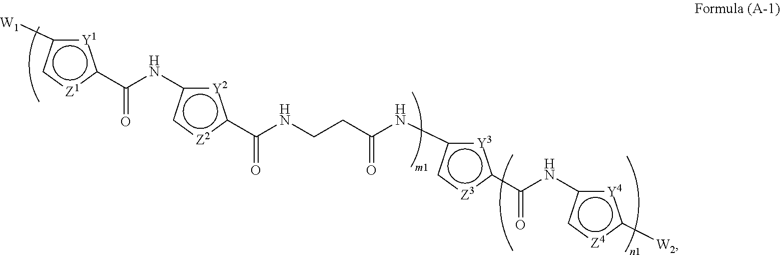

wherein;
m₁ is 1-4;
n₁ is 0-2;
each Y¹, Y², Y³, and Y⁴ is independently CH or N;
each Z¹, Z², Z³, and Z⁴ is independently O, S. or NR²; W₁ is hydrogen, deuterium, halogen, optionally substituted C₁-C₁₀ alkyl, —NR^{1e}C(O)NR^{1e}R^{1f}, —C(O)NR^{1e}R^{1f}, —O—C(O)NR^{1e}R^{1f}, —NR^{1e}C(O)—OR^{1f}, or (AA)₁₋₁₀;
W₂ is hydrogen, deuterium, halogen, optionally substituted C₁-C₁₀ alkyl, —C(O)NR^{1e}R^{1f}, or (AA)₁₋₁₀; wherein
   each R^{1e} is independently hydrogen, optionally substituted C₁-C₅₀ alkyl, optionally substituted C₁-C₅₀ alkenyl, optionally substituted C₁-C₅₀ alkynyl, optionally substituted C₁-C₅₀ heteroalkyl, optionally substituted C₁-C₅₀ heteroalkenyl, optionally substituted C₁₋₅₀ heteroalkynyl, or PEG₁₋₅₀;
   each R^{1f} is independently hydrogen, optionally substituted C₁-C₂₀ alkyl, optionally substituted C₁-C₂₀ alkenyl, optionally substituted C₁-C₂₀ alkynyl, optionally substituted C₁-C₂₀ heteroalkyl, optionally substituted C₁-C₂₀ heteroalkenyl, optionally substituted C₁-C₂₀ heteroalkynyl, PEG₁₋₂₀, or one or more AA, wherein each AA is independently a naturally occurring amino acid;
   or R^{1e} and R^{1f} together with the nitrogen atom which they are attached form an optionally substituted heterocycloalkyl; and
each R² is independently hydrogen, optionally substituted C₁-C₅₀ alkyl, optionally substituted C₁-C₅₀ alkenyl, optionally substituted C₁-C₅₀ alkynyl, optionally substituted C₁-C₅₀ heteroalkyl, optionally substituted C₁-C₅₀ heteroalkyl, C₁-C₅₀ heteroalkenyl, C₁-C₅₀ heteroalkynyl, C₃-C₈ cycloalkyl or 3 to 8-membered heterocycloalkyl, or PEG₁₋₅₀.

In some embodiments, the linker moiety is connected to the DNA binding moiety (i.e., a polyamide) at W₂. In some embodiments, W₂ is optionally substituted C₁-C₆ alkyl, —C(O)NR^{1e}R^{1f}, or (AA)₁₋₁₀. In some embodiments, W₂ is a —C(O)NR^{1e}R^{1f}. In some embodiments, W₂ is —C(O)NH(CH₂)₂C(O)—.

In some embodiments, W₂ is hydrogen.

In some embodiments, W₂ is (AA)₁₋₁₀. In some embodiments, each AA is independently β-alanine.

In some embodiments, the first terminus comprises a polyamide having the structure of Formula (A-2), or a pharmaceutically acceptable salt thereof:

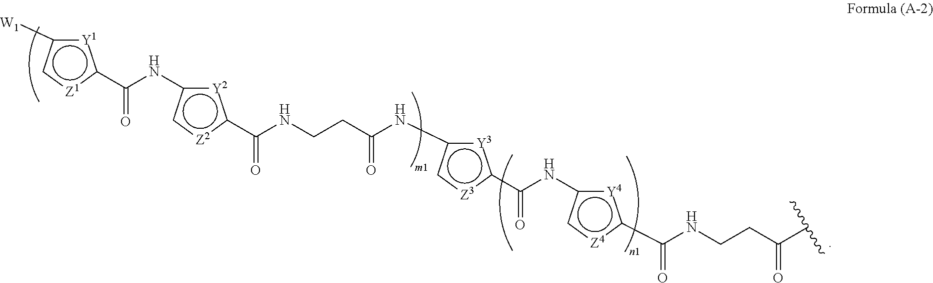

In some embodiments, each R^{1e} and R^{1f} is independently hydrogen, optionally substituted C₁-C₂₀ alkyl, optionally substituted C₁-C₂₀ heteroalkyl, or PEG₁₋₂₀. In some embodiments, each R^{1e} and R^{1f} is independently hydrogen, optionally substituted C₁-C₁₀ alkyl, optionally substituted C₁-C₁₀ heteroalkyl, or PEG₁₋₂₀.

In some embodiments, each R^{1e} is independently optionally substituted C₁-C₂₀ alkyl, optionally substituted C₁-C₂₀ heteroalkyl, or PEG₁₋₂₀, each of which is optionally substituted with amido, alkyl, alkynyl, azido, amino, halogen, haloalkyl, hydroxy, nitro, oxo (═O), phosphorous hydroxide, or PEG. In some embodiments, each R^{1e} is independently optionally substituted C₁-C₂₀, optionally substituted with —CN, —NH₂, —N₃, —OH, CF₃, —OP(O)(OH)₂, —OP(O)(OCH₃)₂, —OP(O)(OCH₃)(OH), or —OP(O)₂OH. In some embodiments, each R^{1e} is independently PEG₁₋₅₀.

In some embodiments, each Z¹, Z², Z³, and Z⁴ is independently NR², wherein R² is optionally substituted C₁-C₂₀ alkyl or optionally substituted C₁-C₂₀ heteroalkyl.

In some embodiments, R^{1e} and R^{1f} together with the nitrogen atom to which they are attached form an optionally substituted heterocycloalkyl.

In some embodiments, each Z¹, Z², Z³, and Z⁴ is independently NCH₃.

In some embodiments, each Z¹, Z², Z³, and Z⁴ is independently NH.

In some embodiments, the first terminus comprises a polyamide having the structure of Formula (A-3), or a pharmaceutically acceptable salt thereof:

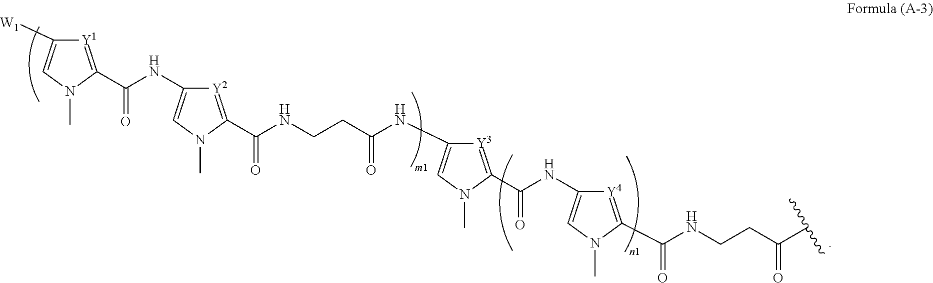

In some embodiments, each Y¹ and Y³ are N; and each Y² and Y⁴ are independently CH or N. In some embodiments, each Y² and Y⁴ is independently CH. In some embodiments, each Y² and Y⁴ is independently N. In some embodiments, Y² is CH and Y⁴ is N. In some embodiments, Y² is N and Y⁴ is CH.

In some embodiments, the linker moiety is connected to the DNA binding moiety through W₁. In some embodiments, W₁ is —C(O)NR^{1e}R^{1f}, wherein R^{1e} is hydrogen; and R^{1f} is hydrogen, optionally substituted C₁-C₁₀ alkyl, or PEG₁₋₂₀.

In some embodiments, W₁ is hydrogen.

In some embodiments, the first terminus comprises a polyamide having the structure of Formula (A-4), or a pharmaceutically acceptable salt thereof:

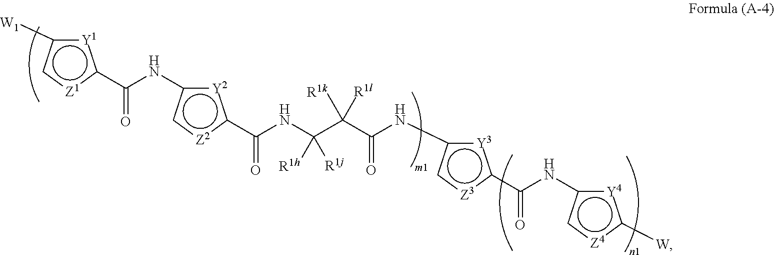

wherein,
each R^{1h}, R^{1j}, R^{1k}, and R^{1l} is independently hydrogen, halogen, —OH, C₁-C₆ alkyl, C₁-C₆ heteroalkyl, C₁-C₆ haloalkyl, or C₁-C₆ hydroxyalkyl; or
R^{1h} and R^{1j} or R^{1l} and R^{1k} combine together with the atom to which they are attached to form a C₃-C₆ cycloalkyl or 4 to 7-membered heterocycloalkyl.

In some embodiments, each R^{1h}, R^{1j}, R^{1k}, and R^{1l} is independently hydrogen, halogen, C₁-C₆ alkyl, C₁-C₆ heteroalkyl, C₁-C₆ haloalkyl, or C₁-C₆ hydroxyalkyl. In some embodiments, each R^{1h}, R^{1j}, R^{1k}, and R^{1l} is independently hydrogen, halogen, or C₁-C₆ alkyl. In some embodiments, each R^{1h}, R^{1j}, R^{1k}, and R^{1l} is independently halogen. In some embodiments, each R^{1h}, R^{1j}, R^{1k}, and R^{1l} is independently C₁-C₆ alkyl. In some embodiments, each R^{1h}, R^{1j}, R^{1k}, and R^{1l} is independently hydrogen.

In some embodiments, R^{1h} and R^{1j} or R^{1l} and R^{1k} combine together with the atom to which they are attached to form an optionally substituted C₃-C₆ cycloalkyl or 4 to 7-membered heterocycloalkyl. In some embodiments, R^{1h} and R^{1j} or R^{1l} and R^{1k} combine together with the atom to which they are attached to form a C₃-C₆ cycloalkyl. In some embodiments, R^{1h} and R^{1j} or R^{1l} and R^{1k} combine together with the atom to which they are attached to form a 4 to 7-membered heterocycloalkyl.

In some embodiments, the first terminus comprises a polyamide having the structure of Formula (A-5), or a pharmaceutically acceptable salt thereof:

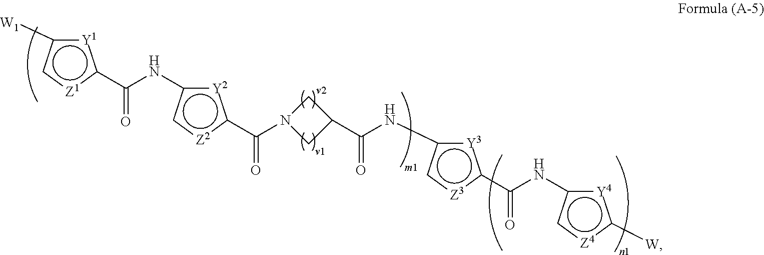

wherein; each v₁ and v₂ are independently 1-3.

In some embodiments, each unit m₁ and n₁ are different or the same. In some embodiments, each unit m₁ is different. In some embodiments, each unit m₁ is the same. In some embodiments, each unit n₁ is different. In some embodiments, each unit n₁ is the same.

In some embodiments, m₁ is 2 or 3; and n₁ is 0 or 1.

In some embodiments, m₁ is 2. In some embodiments, m₁ is 1.

In some embodiments, n₁ is 0. In some embodiments, n₁ is 1.

In some embodiments, each v₁ is independently 1. In some embodiments, each v₁ is independently 2. In some embodiments, each v₁ is independent 3. In some embodiments, each v₂ is independently 1. In some embodiments, each v₂ is independently 2. In some embodiments, each v₂ is independently 3.

In some embodiments, the first terminus comprises a polyamide having the structure of Formula (A-6), or a pharmaceutically acceptable salt thereof:

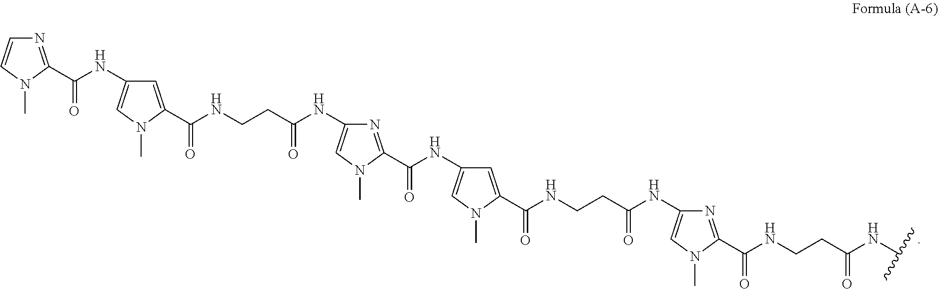 [SMILES: *NC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C]

In some embodiments, the first terminus comprises a polyamide having the structure of Formula (A-7) or a pharmaceutically acceptable salt thereof:

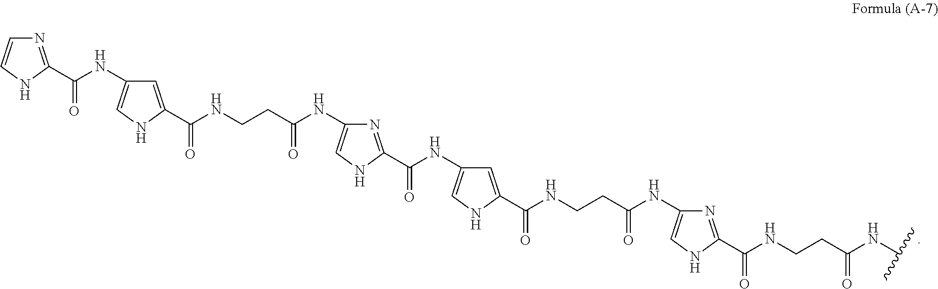 [SMILES: *NC(=O)CCNC(=O)c1nc(NC(=O)CCNC(=O)c2cc(NC(=O)c3nc(NC(=O)CCNC(=O)c4cc(NC(=O)c5ncc[nH]5)c[nH]4)c[nH]3)c[nH]2)c[nH]1]

In some embodiments, the first terminus comprises a polyamide having the structure of Formula (A-8), or a pharmaceutically acceptable salt thereof:

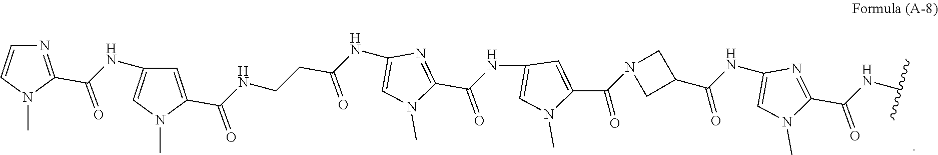 [SMILES: *NC(=O)c1nc(NC(=O)C2CN(C(=O)c3cc(NC(=O)c4nc(NC(=O)CCNC(=O)c5cc(NC(=O)c6nccn6C)cn5C)cn4C)cn3C)C2)cn1C]

The first terminus in the compounds described herein has a high binding affinity to a sequence having multiple repeats of GAA and binds to the target nucleotide repeats preferentially over other nucleotide repeats or nucleotide sequences. In some embodiments, the first terminus has a higher binding affinity to a sequence having multiple repeats of GAA than to a sequence having repeats of CGG. In some embodiments, the first terminus has a higher binding affinity to a sequence having multiple repeats of GAA than to a sequence having repeats of CCG. In some embodiments, the first terminus has a higher binding affinity to a sequence having multiple repeats of GAA than to a sequence having repeats of CCTG. In some embodiments, the first terminus has a higher binding affinity to a sequence having multiple repeats of GAA than to a sequence having repeats of TGGAA. In some embodiments, the first terminus has a higher binding affinity to a sequence having multiple repeats of GAA than to a sequence having repeats of GGGGCC. In some embodiments, the first terminus has a higher binding affinity to a sequence having multiple repeats of GAA than to a sequence having repeats of CAG. In some embodiments, the first terminus has a higher binding affinity to a sequence having multiple repeats of GAA than to a sequence having repeats of CTG.

Due to the preferential binding between the first terminus and the target nucleotide repeat, the transcription modulation molecules described herein become localized around regions having multiple repeats of GAA. In some embodiments, the local concentration of the first terminus or the molecules described herein is higher near a sequence having multiple repeats of GAA than near a sequence having repeats of CGG. In some embodiments, the local concentration of the first terminus or the molecules described herein is higher near a sequence having multiple repeats of GAA than near a sequence having repeats of CCG. In some embodiments, the local concentration of the first terminus or the molecules described herein is higher near a sequence having multiple repeats of GAA than near a sequence having repeats of CCTG. In some embodiments, the local concentration of the first terminus or the molecules described herein is higher near a sequence having multiple repeats of GAA than near a sequence having repeats of TGGAA. In some embodiments, the local concentration of the first terminus or the molecules described herein is higher near a sequence having multiple repeats of GAA than near a sequence having repeats of GGGGCC. In some embodiments, the local concentration of the first terminus or the molecules described herein is higher near a sequence having multiple repeats of GAA than near a sequence having repeats of CTG. In some embodiments, the local concentration of the first terminus or the molecules described herein is higher near a sequence having multiple repeats of GAA than near a sequence having repeats of CAG.

The first terminus is localized to a sequence having multiple repeats of GAA and binds to the target nucleotide repeats preferentially over other nucleotide repeats. In some embodiments, the sequence has at least 2, 3, 4, 5, 8, 10, 12, 15, 20, 25, 30, 40, 50, 100, 200, 300, 400, or 500 repeats of GAA. In some embodiments, the sequence comprises at least 1000 nucleotide repeats of GAA. In some embodiments, the sequence comprises at least 500 nucleotide repeats of GAA. In some embodiments, the sequence comprises at least 200 nucleotide repeats of GAA. In some embodiments, the sequence comprises at least 100 nucleotide repeats of GAA. In some embodiments, the sequence comprises at least 50 nucleotide repeats of GAA. In some embodiments, the sequence comprises at least 20 nucleotide repeats of GAA.

In an aspect, the compounds of the present disclosure can bind to the repeated GAA of fxn than to GAA elsewhere in the subject's DNA

The polyamide composed of a pre-selected combination of subunits can selectively bind to the DNA in the minor groove. In their hairpin structure, antiparallel side-by-side pairings of two aromatic amino acids bind to DNA sequences, with a polyamide ring selected specifically against each DNA base. N-Methylpyrrole (Py) favors T, A, and C bases, excluding G; N-methylimidazole (Tm) is a G-reader; and 3-hydroxyl-N-methylpyrrol (Hp) is specific for thymine base. The nucleotide base pairs can be recognized using different pairings of the amino acid subunits using the paring principle shown in Table 1A and 1 below. For example, an Im/Py pairing reads G·C by symmetry, a Py/Im pairing reads C·G, an Hp/Py pairing can distinguish T·A from A·T, G·C, and C·G, and a Py/Py pairing nonspecifically discriminates both A·T and T·A from G·C and C·G.

In some embodiments, the first terminus comprises Im corresponding to the nucleotide G; Py or beta corresponding to the nucleotide A; Py corresponding to the nucleotide A, wherein Im is N-alkyl imidazole, Py is N-alkyl pyrrole, and beta is β-alanine. In some embodiments, the first terminus comprises Im/Py to correspond to the nucleotide pair G/C, Py/beta or Py/Py to correspond to the nucleotide pair A/T, and wherein Im is N-alkyl imidazole (e.g., N-methyl imidazole), Py is N-alkyl pyrrole (e.g., N-methyl pyrrole), and beta is β-alanine.

TABLE 1A
Base paring for single amino acid subunit (Favored (+), disfavored (−).

| Subunit | G | C | A | T |
|---|---|---|---|---|
| Py | − | + | + | + |
| Im | + | − | − | |
| Hp 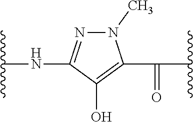 [SMILES: *Nc1nn(C)c(C(*)=O)c1O] | − | − | − | + |
| Th 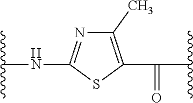 [SMILES: *Nc1nc(C)c(C(*)=O)s1] | − | − | + | + |
| Pz 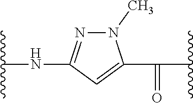 [SMILES: *Nc1cc(C(*)=O)n(C)n1] | − | − | + | + |
| Tp 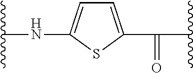 [SMILES: *Nc1ccc(C(*)=O)s1] | − | − | + | + |
| Nt 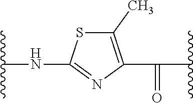 [SMILES: *Nc1nc(C(*)=O)c(C)s1] | + | − | − | − |
| Ht 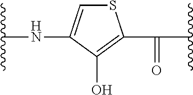 [SMILES: *Nc1csc(C(*)=O)c1O] | − | − | − | + |
| iPTA 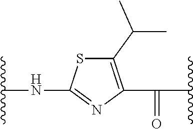 [SMILES: *Nc1nc(C(*)=O)c(C(C)C)s1] | + | − | − | − |
| CTh 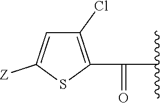 | − | − | − | + |
| PEG 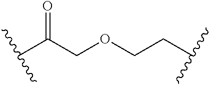 [SMILES: *CCOCC(*)=O] | − | + | + | + |
| iIm 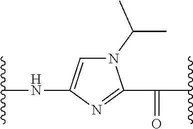 [SMILES: *Nc1cn(C(C)C)c(C(*)=O)n1] | + | − | − | − |
| Ip 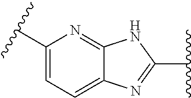 [SMILES: *c1ccc2nc(*)[nH]c2n1] | + | − | − | − |
| Hz 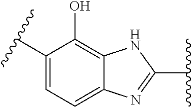 [SMILES: *c1nc2ccc(*)c(O)c2[nH]1] | − | − | − | + |
| Bi 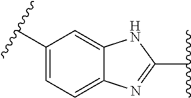 [SMILES: *c1ccc2nc(*)[nH]c2c1] | − | − | − | + |
| Gly 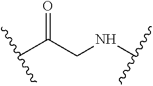 [SMILES: *NCC(*)=O] | − | − | − | − |
| B or bcta 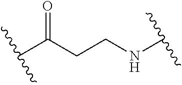 [SMILES: *NCCC(*)=O] | − | − | + | + |
| gAB 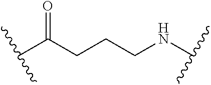 [SMILES: *NCCCC(*)=O] | − | − | + (as a part of the turn) | + (as a part of the turn) |
| Alx 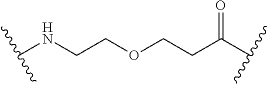 [SMILES: *NCCOCCC(*)=O] | − | + | − | − |
| Da 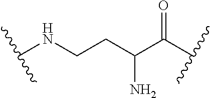 [SMILES: *NCCC(N)C(*)=O] | − | − | + | + |
| Dp 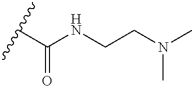 [SMILES: *C(=O)NCCN(C)C] | − | − | + | + |
| iPP 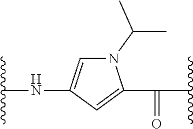 [SMILES: *Nc1cc(C(*)=O)n(C(C)C)c1] | − | − | + | + |
| CTh 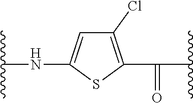 [SMILES: *Nc1cc(Cl)c(C(*)=O)s1] | + | + | − | − |
| Dab 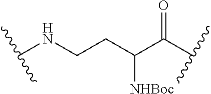 [SMILES: *NCCC(NC(=O)OC(C)(C)C)C(*)=O] | − | − | + | + |
| gAH 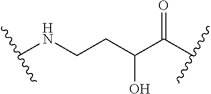 [SMILES: *NCCC(O)C(*)=O] | − | − | + | + |

| | |
|---|---|
| HpBi 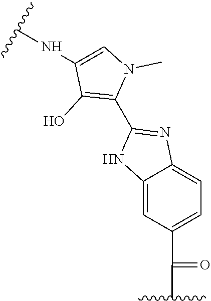 [SMILES: *Nc1cn(C)c(-c2nc3ccc(C(*)=O)cc3[nH]2)c1O] | WW* (bind to two nucleotides with same selectivity as Hp—Py) |
| PyBi 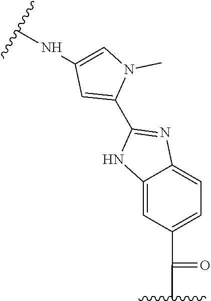 [SMILES: *Nc1cc(-c2nc3ccc(C(*)=O)cc3[nH]2)n(C)c1] | WW* (bind to two nucleotides with same selectivity as Py—Py) |
| ImBi 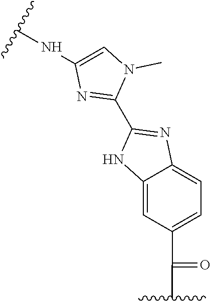 [SMILES: *Nc1cn(C)c(-c2nc3ccc(C(*)=O)cc3[nH]2)n1] | GW* (bind to two nucleotides with same selectivity as Im—Py) |

*The subunit HpBi, ImBi, and PyBi function as a conjugate of two monomer subunits and bind to two nucleotides. The binding property of HpBi, ImBi, and PyBi corresponds to Hp—Py, Im—Py, and Py—Py respectively.

TABLE 1B
Base pairing for hairpin polyamide.

| | G•C | C•G | T•A | A•T |
|---|---|---|---|---|
| Im/β | + | − | − | − |
| β/Im | − | + | − | − |
| Py/β | − | − | + | + |
| β/Py | − | − | + | + |
| β/β | − | − | + | + |
| Py/Py | − | − | + | + |
| Im/Im | − | − | − | − |
| Im/Py | + | − | − | − |
| Py/Im | − | + | − | − |
| Th/Py | − | − | + | − |
| Py/Th | − | − | − | + |
| Th/Im | + | − | − | − |
| Im/Th | − | + | − | − |
| β/Th | − | − | + | − |
| Th/β | − | − | − | + |
| Hp/Py, | − | − | + | − |
| Py/Hp, | − | − | − | + |
| Hp/Im | + | − | − | − |
| Im/Hp | − | + | − | − |
| Tn/Py | − | − | + | + |
| Py/Tn, | − | − | + | + |
| Ht/Py, | − | − | + | + |
| Py/Ht, | − | − | + | + |
| Bi/Py, | − | − | + | + |
| Py/Bi, | − | − | + | + |
| β/Bi | − | − | + | + |
| Bi/β | − | − | + | + |
| Bi/Im, | − | + | − | − |
| Im/Bi, | + | − | − | − |
| Tp/Py, | − | − | + | + |
| Py/Tp, | − | − | + | + |
| β/Tp | − | − | + | + |
| Tp/β | − | − | + | + |
| Tp/Im, | − | + | − | − |
| Im/Tp | + | − | − | − |
| Tp/Tp | − | − | + | + |
| Tp/Tn | − | − | + | + |
| Tn/Tp | − | − | + | + |
| Hz/Py, | − | − | + | − |
| Py/Hz, | − | − | − | + |
| Ip/Py | + | − | − | − |
| Py/Ip, | − | + | − | − |
| Bi/Hz, | − | − | + | + |
| Hz/Bi, | − | − | + | + |
| Bi/Bi | − | + | + | + |
| Th/Py, | − | − | + | + |
| Py/Th | − | − | + | + |
| Im/gAB | + | − | − | − |
| gAB/Im | − | + | − | − |
| Py/gAB | + | − | − | − |
| gAB/Py | − | + | − | − |
| gAB/β | − | − | + | + |
| β/gAB | − | − | + | + |
| Im/Dp | + | − | − | − |
| Dp/Im | − | + | − | − |
| Py/Dp | − | − | + | + |
| Dp/Py | − | − | + | + |
| Dp/β | − | − | + | + |

Each of HpBi, ImBi, and PyBi can bind to two nucleotides and have binding properties corresponding to Hp-Py, Im-Py, and Py-Py respectively. HpBi, ImBi, and PyBi can be paired with two monomer subunits or with themselves in a hairpin structure to bind to two nucleotide pairs.

The monomer subunits of the polyamide can be strung together based on the paring principles shown in Table 1A and Table 1B. The monomer subunits of the polyamide can be strung together based on the paring principles shown in Table 1C.

Table 1C shows an example of the monomer subunits that can bind to the specific nucleotide. The first terminus can include a polyamide described as having several monomer subunits strung together, with a monomer subunit selected from each row. For example, the polyamide can include Im-β-Py that binds to GAA, with Im selected from the first G column, β from the A column, and Py from the second A column. The polyamide can be any combinations that bind to the subunits of GAA, with a subunit selected from each column in Table 1C, wherein the subunits are strung together following the GAA order.

The polyamide can include monomer subunits that bind to 2, 3, 4, or 5 nucleotides of GAA. For example, the polyamide can bind to GA, AA, GAA, AAG, AGA, GAAG, AAGA, GAAGA or GAAGAA. The polyamide can include monomer subunits that bind to 3, 4, 5, 6, 7, 8, 9, or 10 nucleotides of GAA repeats.

The monomer subunit, when positioned as a terminal unit, does not have an amine, carbonyl, or a carboxylic acid group at the terminal. The carboxylic acid group in the terminal is replaced by a hydrogen.

For example, Py, when used as a terminal unit, is understood to have the structure of

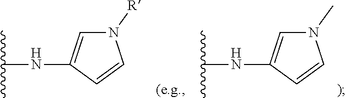

and Im, when positioned as a terminal unit, is understood to have the structure of

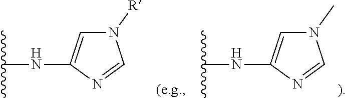

The linear polyamide can have nonlimiting examples including but not limited β-Py-Im, Im-Py-β-Im-Py-β-Im-Py, Im-Py-β-Im-Py-Py-Im-β, Im-Py-Py-Im-Py-β-Im-β, and any combinations thereof.

TABLE 1C
Examples of monomer subunits in a
linear polyamide that binds to GAA.

| Nucleotide | G | A | A |
|---|---|---|---|
| Subunit that selectively binds to nucleotide | Im or ImT | Py | Py |
| | iIm or iImT | Th | Th |
| | PEG | Pz | Pz |
| | CTh | Tp | Tp |
| | Nt | PEG | PEG |
| | iPTA | β | β |
| | Ip | iPP | iPP |
| | CTh | Da | Da |
| | | Dp | Dp |
| | | Dab | Dab |
| | | gAH | gAH |

### Second Terminus—Regulatory Protein Binding Moiety

In some embodiments, the second terminus comprises a protein-binding moiety capable of binding to a regulatory molecule that modulates expression of a gene having the expanded GAA repeat.

In some embodiments, the second terminus comprises a bromodomain binding moiety.

In some embodiments, the second terminus comprises a moiety capable of binding to a bromodomain and extra terminal domain (BET) family member. In some embodiments, the second terminus comprises a moiety capable of binding to an extra terminal domain (BET) family member.

In some embodiments, the BET family member is BRD2, BRD3, BRD4, or BRDT. In some embodiments, the BET family member is BRD2. In some embodiments, the BET family member is BRD3. In some embodiments, the BET family member is BRD4.

In some embodiments, the second terminus comprises a moiety capable of binding to a bromodomain and extra terminal domain (BET) family member, wherein the BET family member is not BRD4.

In some embodiments, the bromodomain is CBP/p300, PCAF (P300/CBP-Associated Factor), CECR2 (cat eye syndrome chromosome region candidate 2), BRPF (bromodomain and PHD finger-containing protein), ATAD2/ATAD2B (chromatin remodeling proteins), TRIM24 (Tripartite motif-containing 24), BAZ2 (Bromodomain Adjacent to Zinc finger), TAF1 (TBP associated factors), BRD 8 (bromodomain-containing protein 8), or BRD 7/9 (bromodomain-containing protein 7, 9).

In some embodiments, the bromodomain is CBP/p300.

In some embodiments, the bromodomain is PCAF (P300/CBP-Associated Factor).

In some embodiments, the bromodomain is CECR2 (cat eye syndrome chromosome region candidate 2).

In some embodiments, the bromodomain is BRPF (bromodomain and PHD finger-containing protein).

In some embodiments, the bromodomain is a ATAD2 or ATAD2B chromatin remodeling protein.

In some embodiments, the bromodomain is BAZ2 (Bromodomain Adjacent Zinc Finger.

In some embodiments, the bromodomain is TAF1 (TBP associated factor).

In some embodiments, the bromodomain is TRIM24 (tripartite motif-containing 24).

In some embodiments, the bromodomain is BRD 8 (bromodomain-containing protein 8).

In some embodiments, the bromodomain is BRD 7/9 (bromodomain-containing protein 7, 9).

In some embodiments, the bromodomain protein is a non-BET bromodomain protein (a bromodomain containing protein that does not belong to the BET protein family).

In some embodiments, the regulatory molecule modulates the rearrangement of histones.

In some embodiments, the regulatory molecule modulates the glycosylation, phosphorylation, alkylation, or acylation of histones.

In some embodiments, the regulatory molecule is a transcription factor.

In some embodiments, the regulatory molecule is an RNA polymerase.

In some embodiments, the regulatory molecule is a moiety that regulates the activity of RNA polymerase.

In some embodiments, the recruiting moiety binds to the regulatory molecule but does not inhibit the activity of the regulatory molecule. In some embodiments, the recruiting moiety binds to the regulatory molecule and inhibits the activity of the regulatory molecule. In some embodiments, the recruiting moiety binds to the regulatory molecule and increases the activity of the regulatory molecule.

In certain embodiments, the recruiting moiety binds to the active site of the regulatory molecule. In certain embodiments, the recruiting moiety binds to a regulatory site of the regulatory molecule.

The binding affinity between the regulatory protein and the second terminus can be adjusted based on the composition of the molecule or type of protein. In some embodiments, the second terminus binds the regulatory molecule with an affinity of less than about 600 nM, about 500 nM, about 400 nM, about 300 nM, about 250 nM, about 200 nM, about 150 nM, about 100 nM, or about 50 nM. In some embodiments, the second terminus binds the regulatory molecule with an affinity of less than about 300 nM. In some embodiments, the second terminus binds the regulatory molecule with an affinity of less than about 200 nM.

In some embodiments, the second terminus comprises Formula (2-A), or a pharmaceutically acceptable salt thereof:

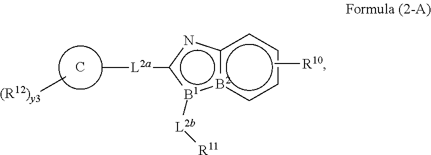

wherein;
Ring C is absent, optionally substituted 6-membered aryl, or optionally substituted 6-membered heteroaryl;
B¹ and B² are each independently C or N, wherein one of B¹ or B² is N;
L^{2a} and L^{2b} are each independently absent, optionally substituted alkylene, —O—, or —NR^{12a}—, wherein
   R^{12a} is hydrogen, deuterium, or optionally substituted C₁-C₁₀ alkyl;
R¹⁰ is an optionally substituted 5 to 6-membered heteroaryl;
R¹¹ is hydrogen or an optionally substituted C₃-C₅ cycloalkyl or 3 to 8-membered heterocycloalkyl; and
each R¹² is independently hydrogen, halogen, —OH, —CN, —NO₂, —NH₂, C₁-C₁₀alkyl, C₁-C₁₀ haloalkyl, or C₁-C₁₀ hydroxyalkyl;
y₃ is 1-4; and
wherein Formula (2-A) is connected to the linker at R¹¹ or one of R¹².

In some embodiments, B¹ is N and B² is C. In some embodiments, B¹ is C and B² is N.

In some embodiments, L²a an optionally substituted alkylene. In some embodiments, L²a is C₁-C₄ alkylene. In some embodiments, L²a is an unsubstituted C₁-C₄ alkylene. In some embodiments, L²a is —O—, or —NR^{12a}—. In some embodiments, L²a is absent.

In some embodiments, Formula (2-A) is connected to the linker through one of R¹². In some embodiments, Formula (2-A) is connected to the linker through R¹¹.

In some embodiments, the second terminus comprises Formula (2-B), or a pharmaceutically acceptable salt thereof:

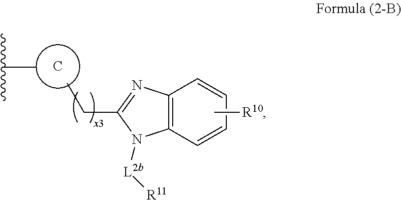

wherein; x₃ is 1-4.

In some embodiments, ring C is an optionally substituted monocyclic 6-membered aryl or optionally substituted 5 to 6-membered heteroaryl. In some embodiments, ring C is an optionally substituted monocyclic 6-membered aryl. In some embodiments, ring C is an optionally substituted phenyl.

In some embodiments, R¹¹ is an optionally substituted C₃-C₅ cycloalkyl. In some embodiments, R¹¹ is optionally substituted 4 to 7 membered heteroaryl. In some embodiments, R¹¹ is hydrogen.

In some embodiments, the second terminus comprises Formula (2-C), or a pharmaceutically acceptable salt thereof:

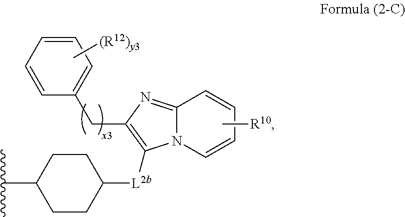

wherein;
R¹⁰ is an optionally substituted 5 to 6-membered heteroaryl;
each R¹² is independently hydrogen, halogen, —OH, —CN, —NO₂, —NH₂, C₁-C₁₀ alkyl, C₁-C₁₀haloalkyl, or C₁-C₁₀ hydroxyalkyl;
x₃ is 1-4; and
y₃ is 1-4.

In some embodiments, L^{2b} an optionally substituted alkylene. In some embodiments, L^{2b} is C₁-C₄ alkylene, optionally substituted with one or more halogen, —OH, —CN, —NO₂, —NH₂, C₁-C₁₀ alkyl, C₁-C₁₀ haloalkyl, or C₁-C₁₀ hydroxyalkyl. In some embodiments, L^{2b} is C₁-C₄ alkylene, optionally substituted with one or more C₁-C₁₀ alkyl, C₁-C₁₀ haloalkyl, or C₁-C₁₀ hydroxyalkyl. In some embodiments, L^{2b} is C₁-C₄ alkylene, optionally substituted with one or more —CH₃, —CH₂CH₃, or —CH(CH₃)₂. In some embodiments. L^{2b} is C₁, C₂, or C₃ alkylene. In some embodiments, L^{2b} is —O— or —NR^{12a}—. In some embodiments, L^{2b} is —NH—. In some embodiments, L²b is absent.

In some embodiments, R¹⁰ is an optionally substituted 5 membered heteroaryl. In some embodiments, R¹⁰ is optionally substituted oxazole, oxadiazole, thiazole, thiadiazole, pyrrole, or pyrazole. In some embodiments, R¹⁰ is optionally substituted oxazole.

In some embodiments, each R¹² is independently halogen, —CN, optionally substituted C₁-C₆ alkyl, optionally substituted C₁-C₆ haloalkyl, or optionally substituted C₁-C₆ hydroxyalkyl. In some embodiments, each R¹² is independently halogen.

In some embodiments, x₃ is an integer from 1. In some embodiments, x₃ is 2. In some embodiments, x₃ is 3. In some embodiments, x₃ is 4.

In some embodiments, y₃ is 1 or 2. In some embodiments, y₄ is 1. In some embodiments, y₃ is 2.

In some embodiments, y₃ is 3. In some embodiments, y₃ is 4.

In some embodiments, the second terminus comprises Formula (2-D) or (2-E), or a pharmaceutically acceptable salt thereof:

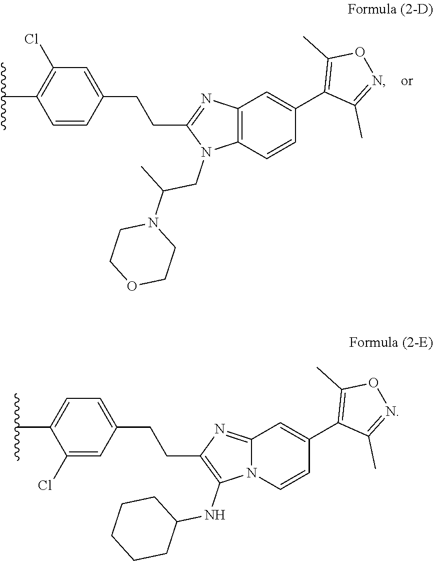 [SMILES: *c1ccc(CCc2nc3cc(-c4c(C)noc4C)ccc3n2CC(C)N2CCOCC2)cc1Cl ; *c1ccc(CCc2nc3cc(-c4c(C)noc4C)ccn3c2NC2CCCCC2)cc1Cl]

In some embodiments, the second terminus comprises Formula (3-A), or a pharmaceutically acceptable salt thereof:

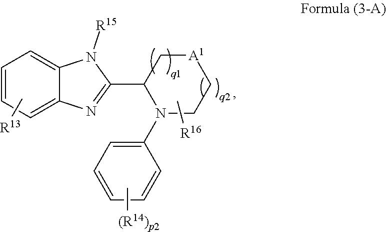

wherein;
A¹ is —CR¹⁷R¹⁷— or —NR¹⁷—; wherein
   R¹⁷ is hydrogen or optionally substituted C₁-C₁₀ alkyl;
R¹³ is an optionally substituted 5 to 6-membered heteroaryl;
each R¹⁴ is independently hydrogen, halogen, C₁-C₆ alkyl, or C₁-C₆ haloalkyl;
R¹⁵ is an optionally substituted C₁-C₁₀ alkyl, optionally substituted C₃-C₈ cycloalkyl, or optionally substituted 3 to 8-membered heterocycloalkyl;
R¹⁶ is hydrogen, halogen, —OH, —CN, —NO₂. —NH₂, oxo (═O), ═S, C₁-C₁₀haloalkyl, or C₁-C₁₀ hydroxyalkyl;
p₂ is 1-4;
q₁ and q₂ are each independently 0-2; and
wherein the linker is attached to Formula (3-A) at either R¹⁵ or R¹⁷.

In some embodiments, R¹³ is an optionally substituted 5-membered heteroaryl. In some embodiments, R¹³ is optionally substituted oxazole, oxadiazole, thiazole, thiadiazole, pyrrole, or pyrazole. In some embodiments, R¹³ is optionally substituted oxazole.

In some embodiments, each R¹⁴ is independently halogen, C₁-C₆ alkyl, or C₁-C₆ haloalkyl. In some embodiments, each R¹⁴ is independently halogen.

In some embodiments, R¹⁵ is an optionally substituted C₁-C₁₀alkyl. In some embodiments, R¹⁵ is an optionally substituted C₃-C₈ cycloalkyl or optionally substituted 3 to 8-membered heterocycloalkyl. In some embodiments, R¹⁵ is a 3- to 8-membered heterocycloalkyl.

In some embodiments, R¹⁶ is hydrogen, halogen, —OH, —CN, —NO₂, —NH₂, C₁-C₁₀ haloalkyl, or C₁-C₁₀ hydroxyalkyl. In some embodiments, R¹⁶ is oxo or ═S. In some embodiments, R¹⁶ is oxo. In some embodiments, R¹⁶ is ═S.

In some embodiments, A¹ is —NR¹⁷. In some embodiments, A¹ is —NH. In some embodiments, A¹ is —NCH₃. In some embodiments, A¹ is —CR¹⁷R¹⁷. In some embodiments, A¹ is —CH₂—.

In some embodiments, R¹⁷ is optionally substituted C₁-C₁₀ alkyl. In some embodiments, R¹⁷ is hydrogen.

In some embodiments, p₂ is 3 or 4. In some embodiments, p₂ is 2. In some embodiments, p₂ is 1.

In some embodiments, q₁ is 1 and q₂ is 1. In some embodiments, q₁ is 2 and q₂ is 0.

In some embodiments, the linker is attached to Formula (3-A) through R¹⁵. In some embodiments, the linker is attached to Formula (3-A) through R⁷.

In some embodiments, the second terminus comprises Formula (3-B), or a pharmaceutically acceptable salt thereof:

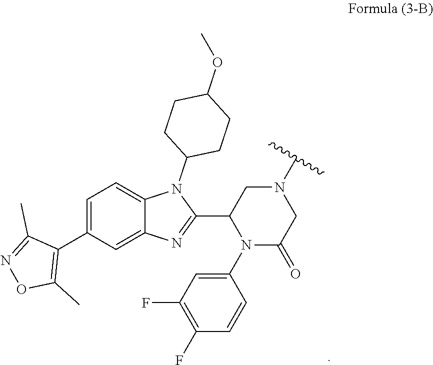 [SMILES: *N1CC(=O)N(c2ccc(F)c(F)c2)C(c2nc3cc(-c4c(C)noc4C)ccc3n2C2CCC(OC)CC2)C1]

In some embodiments, the second terminus comprises Formula (3-C), or a pharmaceutically acceptable salt thereof:

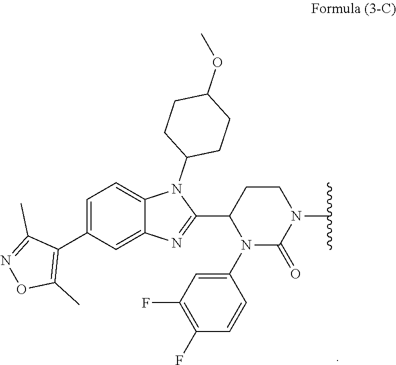 [SMILES: *N1CCC(c2nc3cc(-c4c(C)noc4C)ccc3n2C2CCC(OC)CC2)N(c2ccc(F)c(F)c2)C1=O]

In some embodiments, the second terminus comprises Formula (3-D), or a pharmaceutically acceptable salt thereof:

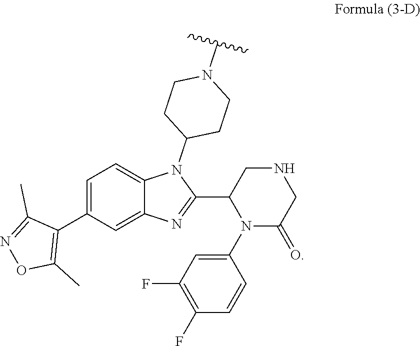 [SMILES: *N1CCC(n2c(C3CNCC(=O)N3c3ccc(F)c(F)c3)nc3cc(-c4c(C)noc4C)ccc32)CC1]

In some embodiments, the second terminus comprises Formula (3-E), or a pharmaceutically acceptable salt thereof:

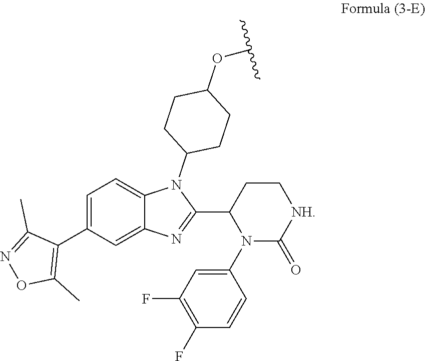 [SMILES: *OC1CCC(n2c(C3CCNC(=O)N3c3ccc(F)c(F)c3)nc3cc(-c4c(C)noc4C)ccc32)CC1]

In some embodiments, the second terminus comprises Formula (4-A), or a pharmaceutically acceptable salt thereof:

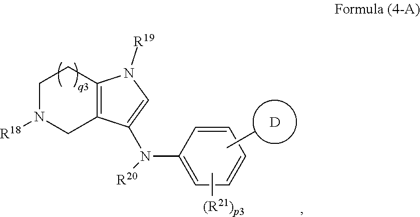

wherein;
ring D is absent or optionally substituted 5 to 6-membered heteroaryl;
R¹⁸ is optionally substituted C₁-C₆ alkyl, optionally substituted C₃-C₈ cycloalkyl, —C(O)R^{18a}, —C(O)—, or —C(O)—NR^{18a}R^{18b}, wherein
   R^{18a} and R^{18b} are each independently an optionally substituted C₁-C₁₀ alkyl or optionally substituted C₃-C₈ cycloalkyl;
R¹⁹ is an optionally substituted C₁-C₁₀ alkyl, optionally substituted C₁-C₁₀ haloalkyl, optionally substituted C₃-C₅ cycloalkyl, or optionally substituted 3 to 8 membered heterocycloalkyl;
R²⁰ is hydrogen or optionally substituted C₁-C₁₀ alkyl;
each R²¹ is independently hydrogen, halogen, —OH, —CN, —NO₂, —NH₂, optionally substituted C₁-C₁₀ alkyl, optionally substituted C₁-C₁₀haloalkyl. C₁-C₁₀ hydroxyalkyl, optionally substituted C₂-C₁₀alkenyl, optionally substituted C₂-C₁₀ alkynyl, optionally substituted C₃-C₅-cycloalkyl, or optionally substituted 3 to 8-membered heterocycle;
or R²⁰ and one of R² together with the atoms to which they are attached form an optionally substituted 5 to 8-membered heterocycloalkyl;
p₃ is 1-4;
q₃ is 0 or 1; and
wherein Formula (4-A) is connected to the linker at ring D or at R¹⁸.

In some embodiments, R¹⁸ is optionally substituted C₁-C₆ alkyl or optionally substituted C₃-C₈ cycloalkyl. In some embodiments, R¹⁸ is —C(O)R^{18a}. In some embodiments, R¹⁸ is —C(O)CH₃ or —C(O)CH₂CH₃. In some embodiments, R¹⁸ is —C(O)—NR^{18a}R^{18b}.

In some embodiments, R^{18a} is optionally substituted C₁-C₁₀ alkyl. In some embodiments, R^{18a} is optionally substituted C₃-C₈ cycloalkyl.

In some embodiments, R^{18b} is optionally substituted C₁-C₁₀ alkyl. In some embodiments, R^{18b} is optionally substituted C₃-C₈ cycloalkyl.

In some embodiments, R¹⁹ is optionally substituted C₁-C₁₀ alkyl or optionally substituted C₁-C₁₀ haloalkyl. In some embodiments, R¹⁹ is optionally substituted C₃-C₈ cycloalkyl or optionally substituted 3 to 8 membered heterocycloalkyl. In some embodiments, R¹⁹ is optionally substituted 3- to 8-membered heterocycloalkyl ring.

In some embodiments, R²⁰ is optionally substituted C₁-C₁₆ alkyl. In some embodiments. R²⁰ is hydrogen.

In some embodiments, each R¹ is independently halogen, —OH, —CN, —NO₂, —NH₂, optionally substituted C₁-C₁₀ alkyl, optionally substituted C₁-C₁₀haloalkyl, C₁-C₁₀ hydroxyalkyl, optionally substituted C₂-C₁₀ alkenyl, optionally substituted C₂-C₁₀ alkynyl, optionally substituted C₃-C₅-cycloalkyl, or optionally substituted 3- to 8-membered heterocycle. In some embodiments, each R²¹ is independently halogen or C₁-C₁₀ haloalkyl.

In some embodiments, R²⁰ and one of R²¹ together with the atoms to which they are attached form an optionally substituted 5 to 8-membered heterocycloalkyl. In some embodiments, R²⁰ and one of R²¹ together with the atoms to which they are attached form a 5, 6, 7, or 8-membered heterocycloalkyl.

In some embodiments, p₃ is 3 or 4. In some embodiments, p₃ is 2. In some embodiments, p₃ is 1.

In some embodiments, q₃ is 1. In some embodiments, q₃ is 0.

In some embodiments, ring D is an optionally substituted 5-membered heteroaryl. In some embodiments, ring D is absent.

In some embodiments, Formula (4-A) is connected to the linker at ring D. In some embodiments, Formula (4-A) is connected to the linker at R¹⁸.

In some embodiments, the second terminus comprises Formula (4-B), or a pharmaceutically acceptable salt thereof:

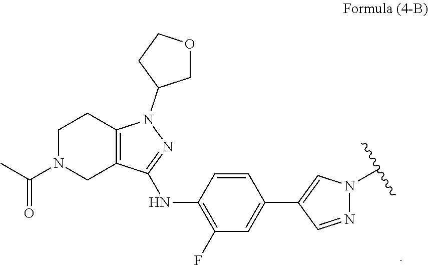 [SMILES: *n1cc(-c2ccc(Nc3nn(C4CCOC4)c4c3CN(C(C)=O)CC4)c(F)c2)cn1]

In some embodiments, the second terminus comprises Formula (4-C1) or Formula (4-C₂), or a pharmaceutically acceptable salt thereof:

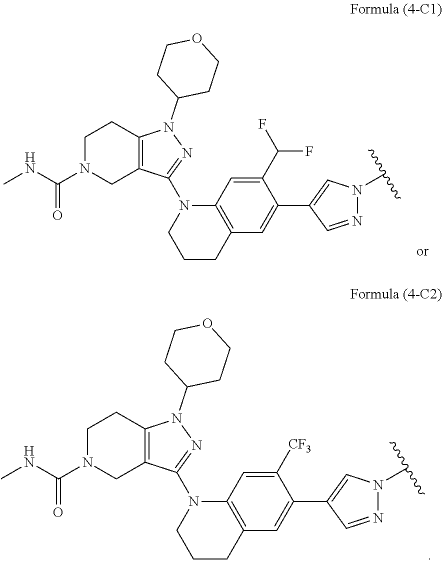 [SMILES: *n1cc(-c2cc3c(cc2C(F)F)N(c2nn(C4CCOCC4)c4c2CN(C(=O)NC)CC4)CCC3)cn1 ; *n1cc(-c2cc3c(cc2C(F)(F)F)N(c2nn(C4CCOCC4)c4c2CN(C(=O)NC)CC4)CCC3)cn1]

In some embodiments, the second terminus comprises Formula (4-D1) or Formula (4-D2), or a pharmaceutically acceptable salt thereof:

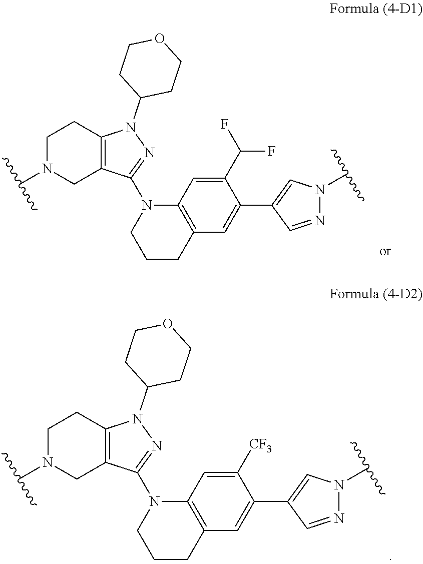 [SMILES: *N1CCc2c(c(N3CCCc4cc(-c5cnn(*)c5)c(C(F)F)cc43)nn2C2CCOCC2)C1 ; *N1CCc2c(c(N3CCCc4cc(-c5cnn(*)c5)c(C(F)(F)F)cc43)nn2C2CCOCC2)C1]

In some embodiments, the second terminus comprises Formula (5-A), or a pharmaceutically acceptable salt thereof:

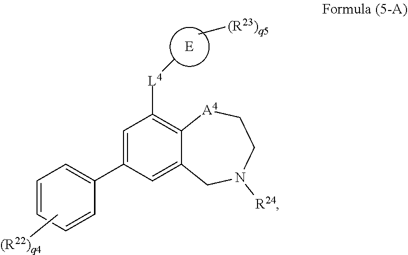

wherein;
ring E is a 5 to 6-membered heterocycloalkyl;
A⁴ is absent, CH₂, —NH—, or —O—;
L⁴ is alkylene or heteroalkylene;
each R²² is independently halogen. —OH, —CN, —NO₂, —NH₂, optionally substituted C₁-C₁₀ alkyl, optionally substituted C₁-C₁₀ haloalkyl, C₁-C₁₀ hydroxyalkyl, optionally substituted C₂-C₁₀ alkenyl, optionally substituted C₂-C₁₀ alkynyl, optionally substituted C₃-C₅-cycloalkyl, or optionally substituted 3 to 8-membered heterocycloalkyl;
each R²³ is independently hydrogen, halogen, —OH, —CN, —NO₂, —NH₂, optionally substituted C₁-C₁₀ alkyl, optionally substituted C₁-C₁₀haloalkyl, optionally substituted C₁-C₁₀ hydroxyalkyl, optionally substituted C₂-C₁₀ alkenyl, optionally substituted C₂-C₁₀ alkynyl, optionally substituted C₃-C₈-cycloalkyl, or optionally substituted 3 to 8-membered heterocycloalkyl;
R²⁴ is optionally substituted C₁-C₁₀ alkyl, —C(O)R^{24a}, or —C(O)—NR^{24a}R^{24b}, wherein R^{24a} and R^{24b} are each independently optionally substituted C₁-C₁₀ alkyl or optionally substituted C₃-C₈ cycloalkyl;
q₄ is 2-3; or
q₅ is 0-2; and
wherein the Formula (5-A) is connected to the linker through ring E or through one of R².

In some embodiments, A⁴ is absent. In some embodiments, A⁴ is —NH— or —O—. In some embodiments, A⁴ is —NH—. In some embodiments, A⁴ is —O—.

In some embodiments, L⁴ is alkylene. In some embodiments, L⁴ is C₁-C₅ alkylene.

In some embodiments, L⁴ is heteroalkylene. In some embodiments, L⁴ is C₁-C₄ heteroalkylene-. In some embodiments, L⁴ is —O—CH₂— or —O—CH₂CH₂—.

In some embodiments, each R²² is independently halogen, —OH, —CN, —NO₂, —NH₂, optionally substituted C₁-C₁₀ alkyl, optionally substituted C₁-C₁₀ haloalkyl, C₁-C₁₀ hydroxyalkyl, optionally substituted C₂-C₁₀ alkenyl, or optionally substituted C₂-C₁₀ alkynyl. In some embodiments, each R²² is independently optionally substituted C₁-C₁₀ alkyl or optionally substituted C₁-C₁₀ hydroxyalkyl. In some embodiments, each R²² is independently C₁-C₁₀ hydroxyalkyl. In some embodiments, each R²² is independently —OCH₃ or —OCH₂CH₃.

In some embodiments, each R²³ is independently hydrogen, halogen, —OH, —CN, —NO₂, —NH₂, optionally substituted C₁-C₁₀ alkyl, optionally substituted C₁-C₁₀haloalkyl, or optionally substituted C₁-C₁₀ hydroxyalkyl. In some embodiments, each R²³ is independently hydrogen.

In some embodiments, R²⁴ is optionally substituted C₁-C₁₀ alkyl. In some embodiments. R²⁴ is —C(O)R^{24a}. In some embodiments, R²⁴ is —C(O)CH₃ or —C(O)CH₂CH₃. In some embodiments, —C(O)—NR^{24a}R^{24b}.

In some embodiments, R^{24a} is optionally substituted C₁-C₁₀ alkyl. In some embodiments, R^{24a} is optionally substituted C₃-C₅ cycloalkyl.

In some embodiments, R^{24b} is optionally substituted C₁-C₁₀ alkyl. In some embodiments, R^{24b} is an optionally substituted C₃-C₅ cycloalkyl.

In some embodiments, ring E is a 6-membered heterocycloalkyl.

In some embodiments, q₄ is 3. In some embodiments, q₄ is 2.

In some embodiments, q₅ is 2. In some embodiments, q₅ is 1. In some embodiments, q₅ is 0.

In some embodiments, Formula (5-A) is connected to the linker through ring E. In some embodiments, Formula (5-A) is connected to the linker through one of R²².

In some embodiments, the second terminus comprises Formula (5-B), or a pharmaceutically acceptable salt thereof:

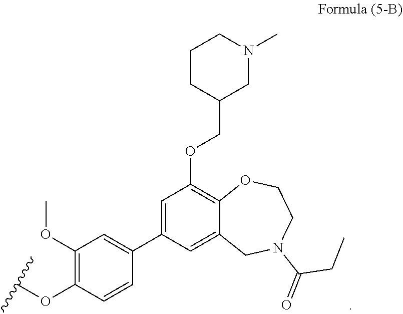 [SMILES: *Oc1ccc(-c2cc3c(c(OCC4CCCN(C)C4)c2)OCCN(C(=O)CC)C3)cc1OC]

In some embodiments, the second terminus comprises Formula (5-C), or a pharmaceutically acceptable salt thereof:

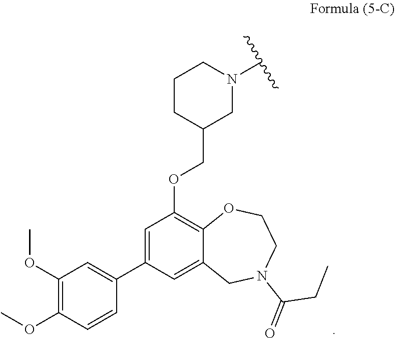 [SMILES: *N1CCCC(COc2cc(-c3ccc(OC)c(OC)c3)cc3c2OCCN(C(=O)CC)C3)C1]

In some embodiments, the second terminus comprises Formula (6-A), or a pharmaceutically acceptable salt thereof:

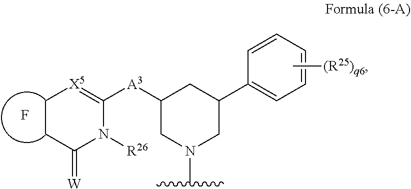

wherein;
ring F is an optionally substituted 5 to 6-membered heteroaryl;
A³ is —O—, —NH—, or —CH₂—;
X⁵ is CH or N;
W is O or S;
each R²⁵ is hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, optionally substituted C₂-C₁₀ alkynyl, optionally substituted C₃-C₅-cycloalkyl, or optionally substituted 3- to 8-membered heterocycloalkyl;
or two R²⁵ together with the atoms to which they are attached form an optionally substituted C₅-C₈ cycloalkyl or optionally substituted 5 to 8-membered heterocycloalkyl;
R²⁶ is hydrogen or optionally substituted C₁-C₁₀ alkyl; and
q₆ is 1-4.

In some embodiments, the second terminus comprises Formula (6-B), or a pharmaceutically acceptable salt thereof:

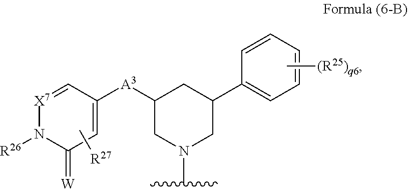

wherein;
A³ is —O—, —NH—, or —CH₂—;
X⁷ is CH or N;
W is O or S;
each R²⁵ is independently hydrogen, halogen, —OH, —CN, —NO₂, —NH₂, optionally substituted C₁-C₁₀ alkyl, optionally substituted C₁-C₁₀haloalkyl, optionally substituted C₁-C₁₀ hydroxyalkyl, optionally substituted C₁-C₁₀ alkenyl, optionally substituted C₂-C₁₀ alkynyl, optionally substituted C₃-C₅-cycloalkyl, or optionally substituted 3 to 8-membered heterocycloalkyl;
or two R²⁵ together with the atoms to which they are attached form an optionally substituted C₅-C₅ cycloalkyl or optionally substituted 5 to 8-membered heterocycloalkyl;
R²⁶ is hydrogen or optionally substituted C₁-C₁₀ alkyl;
R²⁷ is hydrogen, halogen, —OH, —CN, —NO₂. —NH₂, optionally substituted C₁-C₁₀ alkyl, optionally substituted C₁-C₁₀ haloalkyl, or optionally substituted C₁-C₁₀ hydroxyalkyl; and
q₆ is 1-4.

In some embodiments, ring F is an optionally substituted 6-membered heteroaryl. In some embodiments, ring F is an optionally substituted 5-membered heteroaryl.

In some embodiments, A³ is —O— or —CH₂—. In some embodiments, A³ is —O—. In some embodiments, A³ is —CH₂—. In some embodiments, A³ is —NH—.

In some embodiments, X⁵ is CH. In some embodiments, X⁵ is N.

In some embodiments, X⁷ is CH. In some embodiments, X⁷ is N.

In some embodiments, W is O. In some embodiments, W is S.

In some embodiments, each R²⁵ is independently hydrogen, halogen, —OH, —CN, —NO₂, —NH₂, optionally substituted C₁-C₁₀ alkyl, optionally substituted C₁-C₁₀haloalkyl, optionally substituted C₁-C₁₀ hydroxyalkyl, optionally substituted C₂-C₁₀ alkenyl, or optionally substituted C₂-C₁₀ alkynyl. In some embodiments, each R²⁵ is independently hydrogen, optionally substituted C₁-C₁₀ alkyl, or optionally substituted C₁-C₁₀ hydroxyalkl. In some embodiments, each R²⁵ is independently C₁-C₁₀ hydroxyalkyl.

In some embodiments, two R²⁵ tother with the atoms to which they are attached form an optionally substituted C₅-C₈ cycloalkyl or 5 to 8-membered heterocycloalkyl. In some embodiments, two R²⁵ together with the atoms to which they are attached form an optionally substituted 5 to 8-membered heterocycloalkyl.

In some embodiments, R²⁶ is an optionally substituted C₁-C₁₀ alkyl. In some embodiments, R²⁶ is —CH₃, CH₂CH₃, or —CH(CH₃)₂.

In some embodiments, R²⁷ is hydrogen, halogen, —OH, —CN, —NO₂, —NH₂, or optionally substituted C₁-C₁₀ alkyl. In some embodiments, R²⁷ is halogen. In some embodiments. R²⁷ is hydrogen.

In some embodiments, q₆ is 3 or 4. In some embodiments, q₆ is 2. In some embodiments, q₆ is 1.

In some embodiments, the second terminus comprise Formula (6-C), or a pharmaceutically acceptable salt thereof:

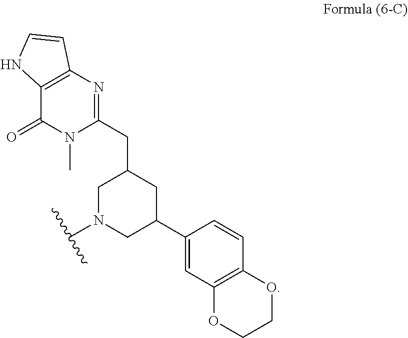 [SMILES: *N1CC(Cc2nc3cc[nH]c3c(=O)n2C)CC(c2ccc3c(c2)OCCO3)C1]

In some embodiments, the second terminus comprises Formula (6-D), or a pharmaceutically acceptable salt thereof:

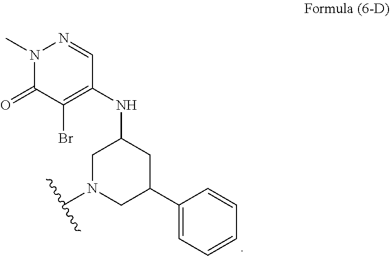 [SMILES: *N1CC(Nc2cnn(C)c(=O)c2Br)CC(c2ccccc2)C1]

In some embodiments, the second terminus comprises Formula (7-A), or a pharmaceutically acceptable salt thereof:

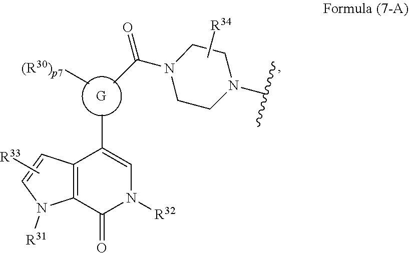

wherein;
ring G is an aryl or heteroaryl;
each R³⁰ is hydrogen, halogen, —OH, —CN, —NO₂, —NH₂, C₁-C₁₀alkyl, C₁-C₁₀haloalkyl, or C₁-C₁₀ hydroxyalkyl;
R³¹ and R³² are each independently hydrogen, optionally substituted C₁-C₁₀ alkyl, optionally substituted C₁-C₁₀ haloalkyl, optionally substituted C₂-C₁₀ alkenyl, or optionally substituted C₂-C₁₀ alkynyl;
R³³ is hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, or optionally substituted C₂-C₁₀ alkynyl;
R³⁴ is hydrogen, halogen, —OH, —CN, —NO₂. —NH₂, optionally substituted C₁-C₁₀ alkyl, optionally substituted C₁-C₁₀ haloalkyl, or optionally substituted C₁-C₁₀ hydroxyalkyl; and
p₇ is 1-4.

In some embodiments, ring G is an aryl In some embodiments, the aryl is phenyl.

In some embodiments, the second terminus comprises Formula (7-B), or a pharmaceutically acceptable salt thereof:

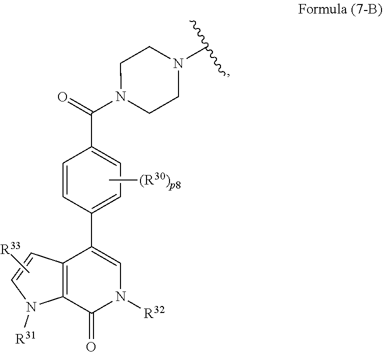

wherein; p₈ is 1-3.

In some embodiments, ring G is a bicyclic heteroaryl comprising 1-2 heteroatoms selected from N, O, or S.

In some embodiments, the second terminus comprises Formula (7-C), or a pharmaceutically acceptable salt thereof:

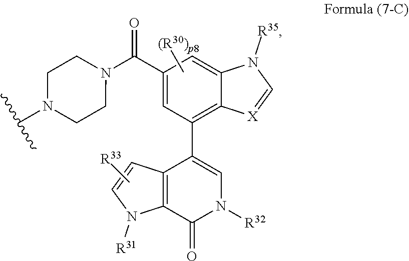

wherein;
X is CR³⁰ or N;
R³⁵ is hydrogen or optionally substituted C₁-C₁₀ alkyl; and
p₈ is 1-3.

In some embodiments, each R³⁰ is hydrogen, halogen, —OH, —CN, —NO₂, —NH₂, C₁-C₁₀ alkyl, C₁-C₁₀ haloalkyl, or optionally substituted C₁-C₁₀ hydroxyalkyl.

In some embodiments, R^{3′} is hydrogen, optionally substituted C₁-C₁₀ alkyl, optionally substituted C₁-C₁₀ haloalkyl, optionally substituted C₂-C₁₀ alkenyl, or optionally substituted C₂-C₁₀ alkynyl. In some embodiments, R³¹ is an optionally substituted C₁-C₁₀ alkyl. In some embodiments, R³¹ is methyl, ethyl, isopropyl, or tert-butyl. In some embodiments, R³¹ is hydrogen.

In some embodiments, R³² is hydrogen, optionally substituted C₁-C₁₀ alkyl, optionally substituted C₁-C₁₀ haloalkyl, optionally substituted C₂-C₁₀ alkenyl, or optionally substituted C₂-C₁₀ alkynyl. In some embodiments, R³² is an optionally substituted C₁-C₁₀ alkyl or optionally substituted C₂-C₁₀ alkenyl. In some embodiments, R³² is hydrogen.

In some embodiments, R³³ is hydrogen, halogen, —OH, —CN, —NO₂, —NH₂, optionally substituted C₁-C₁₀ alkyl, optionally substituted C₁-C₁₀haloalkyl, optionally substituted C₁-C₁₀ hydroxyalkyl, optionally substituted C₂-C₁₀ alkenyl, or optionally substituted C₂-C₁₀ alkynyl.

In some embodiments, R³⁴ is hydrogen, halogen, —OH, —CN, —NO₂, —NH₂, optionally substituted C₁-C₁₀ alkyl, optionally substituted C₁-C₁₀ haloalkyl, or optionally substituted C₁-C₁₀ hydroxyalkyl.

In some embodiments, R³⁴ is hydrogen.

In some embodiments, R¹⁵ is hydrogen or optionally substituted C₁-C₁₀ alkyl. In some embodiments, R³⁵ is an optionally substituted C₁-C₁₀ alkyl. In some embodiments, R³⁵ is methyl, ethyl, isopropyl, or tert-butyl. In some embodiments, R³⁵ is hydrogen.

In some embodiments, p₇ is 4. In some embodiments, p₇ is 3. In some embodiments, p₇ is 2. In some embodiments, p₇ is 1.

In some embodiments, the second terminus comprises Formula (7-D1), (7-D2), or (7-D3), or a pharmaceutically acceptable salt thereof:

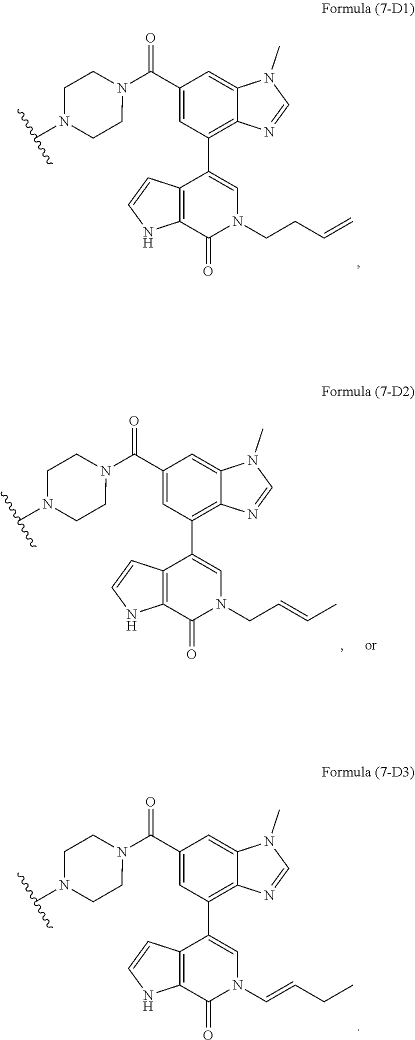 [SMILES: *N1CCN(C(=O)c2cc(-c3cn(CCC=C)c(=O)c4[nH]ccc34)c3ncn(C)c3c2)CC1 ; *N1CCN(C(=O)c2cc(-c3cn(C/C=C/C)c(=O)c4[nH]ccc34)c3ncn(C)c3c2)CC1 ; *N1CCN(C(=O)c2cc(-c3cn(/C=C/CC)c(=O)c4[nH]ccc34)c3ncn(C)c3c2)CC1]

In some embodiments, the second terminus comprises Formula (7-E1), (7-E2), or (7-E3), or a pharmaceutically acceptable salt thereof:

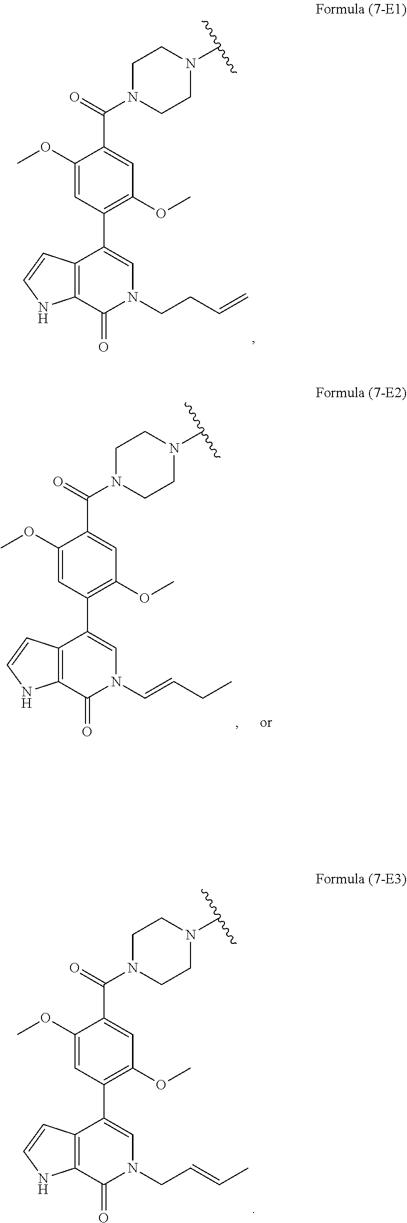

In some embodiments, the second terminus comprises Formula (8-A), or a pharmaceutically acceptable salt thereof:

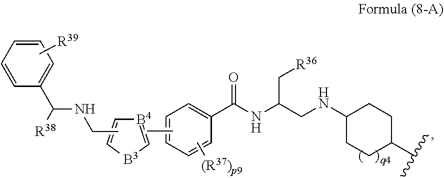

wherein;
B³ is —O—, —NH—, or S;
B⁴ is N or CH;
R³⁶ is an optionally substituted aryl or heteroaryl;
each R³⁷ is independently halogen, —OH, —CN, —NO₂, —NH₂, optionally substituted C₁-C₁₀ alkyl, optionally substituted C₁-C₁₀ haloalkyl, or optionally substituted C₁-C₁₀ hydroxyalkl;
R³⁸ is hydrogen, optionally substituted C₁-C₁₀ alkyl, optionally substituted C₁-C₁₀ haloalkyl, or optionally substituted C₁-C₁₀ hydroxyalkyl;
R³⁹ is halogen, —OH, —CN, —NO₂, —NH₂, optionally substituted C₁-C₁₀ alkyl, optionally substituted C₁-C₁₀ haloalkyl, or optionally substituted C₁-C₁₀ hydroxyalkyl;
p₉ is 1-3; and
q₄ is 0-2.

In some embodiments, B³ is —O— or —S—. In some embodiments. B³ is —O—. In some embodiments, B³ is —S—.

In some embodiments, B⁴ is N. In some embodiments, B⁴ is CH.

In some embodiments, R³⁶ is an optionally substituted aryl. In some embodiments, R³⁶ is phenyl optionally substituted with one or more halogen, —CN, —NH₂, —OH, C₁-C₁₀ alkyl, C₁-C₁₀ haloalkyl, or C₁-C₁₀ hydroxyalkyl.

In some embodiments, each R³⁷ is independently halogen, —OH, —CN, —NH₂, optionally substituted C₁-C₁₀ alkyl, optionally substituted C₁-C₁₀ haloalkyl, or optionally substituted C₁-C₁₀ hydroxyalkyl.

In some embodiments, R³⁸ is optionally substituted C₁-C₁₀ alkyl, optionally substituted C₁-C₁₀ haloalkyl, or optionally substituted C₁-C₁₀ hydroxyalkyl. In some embodiments, R³³ is optionally substituted C₁-C₁₀ alkyl.

In some embodiments, R³⁹ is halogen, —OH, —CN, —NO₂, —NH₂, or optionally substituted C₁-C₁₀ alkyl.

In some embodiments, p₉ is 3. In some embodiments, p₉ is 2. In some embodiments, p₉ is 1.

In some embodiments, q₄ is 2. In some embodiments, q₄ is 1. In some embodiments, q₄ is 0.

In some embodiments, the second terminus comprises Formula (8-B), or a pharmaceutically acceptable salt thereof:

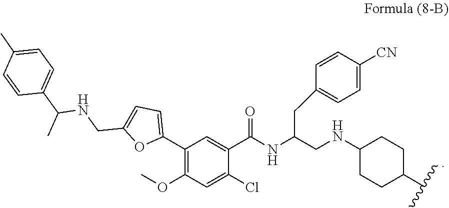 [SMILES: *C1CCC(NCC(Cc2ccc(C#N)cc2)NC(=O)c2cc(-c3ccc(CNC(C)c4ccc(C)cc4)o3)c(OC)cc2Cl)CC1]

In some embodiments, the second terminus comprises Formula (9-A), or a pharmaceutically acceptable salt thereof:

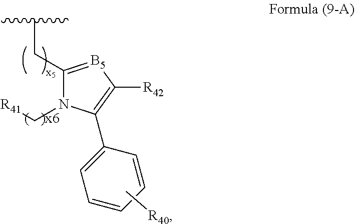

wherein:
B⁵ is N or CH;
R⁴⁰ is hydrogen, halogen, —OH, —CN, —NO₂, —NH₂, C₁-C₁₀ alkyl, C₁-C₁₀ haloalkyl, or C₁-C₁₀ hydroxyalkyl;
R⁴¹ and R⁴² are each independently an optionally substituted 5-membered heteroaryl; and
x₅ and x₆ are each independently 0-4.

In some embodiments, B⁵ is N. In some embodiments, B⁵ is CH.

In some embodiments, R⁴⁰ is halogen, —OH, —CN, —NO₂, —NH₂, C₁-C₁₀ alkyl, C₁-C₁₀ haloalkyl, or C₁-C₁₀ hydroxyalkyl. In some embodiments, R⁴⁰ is halogen, —OH, —CN, —NO₂, —NH₂, or —CH₃.

In some embodiments, R⁴¹ is optionally substituted oxazole, oxadiazole, thiazole, thiadiazole, pyrrole, or pyrazole. In some embodiments, R⁴¹ is pyrrole or pyrazole.

In some embodiments, R⁴² is optionally substituted oxazole, oxadiazole, thiazole, thiadiazole, pyrrole, or pyrazole. In some embodiments, R⁴² is pyrrole or pyrazole.

In some embodiments, x₅ is 2 or 3. In some embodiments, x; is 1. In some embodiments, x₅ is 0.

In some embodiments, x₆ is 3. In some embodiments, x₆ is 2. In some embodiments, x₆ is 1. In some embodiments, x₆ is 0.

In some embodiments, the second terminus comprise Formula (9-B), or a pharmaceutically acceptable salt thereof:

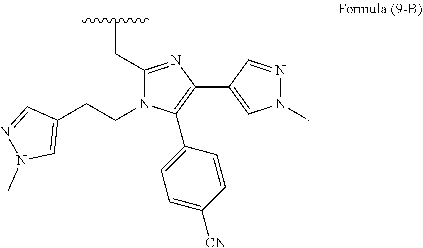

In some embodiments, the second terminus comprises Formula (10-A), or a pharmaceutically acceptable salt thereof:

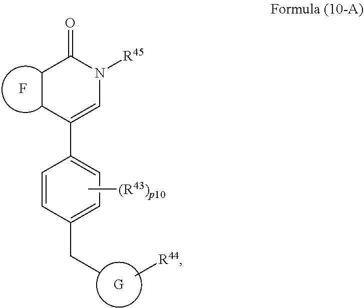

wherein;
ring F is aryl or heteroaryl;
ring G is absent or a 4 to 8-membered heterocycloalkyl; A⁵ is —O—, —NH—, or —CH₂—;
each R⁴³ is independently halogen. —OH, —CN, —NO₂, —NH₂, C₁-C₁₀ alkyl, C₁-C₁₀haloalkyl, or C₁-C₁₀ hydroxyalkyl;
R⁴⁴ is hydrogen, —OH, —NH₂, C₁-C₁₀ alkyl, C₁-C₁₀ haloalkyl, C₁-C₁₀ hydroxyalkyl, or —NH—C₁-C₁₀ alkyl;
R⁴⁵ is hydrogen or optionally substituted C₁-C₁₀ alkyl; and
p₁₀ is 1-4; and
wherein Formula (11-A) is connected to the linker through R⁴⁴.

In some embodiments, ring F is an aryl, optionally substituted with one or more halogen, CN, NH₂, OH, C₁-C₁₀ alkyl, C₁-C₁₀ haloalkyl, or C₁-C₁₀ hydroxyalkyl. In some embodiments, ring F is phenyl. In some embodiments, ring F is an optionally substituted 6-membered heteroaryl, optionally substituted with one or more halogen, CN, NH₂, OH, C₁-C₁₀ alkyl, C₁-C₁₀haloalkyl, or C₁-C₁₀ hydroxyalkyl. In some embodiments, ring F is an optionally substituted pyridine.

In some embodiments, ring G is a 4 to 8-membered heterocycloalkyl. In some embodiments, ring G is a 4-membered heterocycloalkyl. In some embodiments, ring G is a 5-membered heterocycloalkyl. In some embodiments, ring G is a 6-membered heterocycloalkyl. In some embodiments, ring G is absent.

In some embodiments, A⁵ is —O— or —NH—. In some embodiments, A⁵ is —CH₂—.

In some embodiments, each R⁴³ is independently —OH, —NH₂, C₁-C₁₀alkyl, C₁-C₁₀haloalkyl, or C₁-C₁₀ hydroxyalkyl. In some embodiments, each R⁴³ is independently C₁-C₁₀ alkyl, or C₁-C₁₀ hydroxyalkyl. In some embodiments, each R⁴³ is independently C₁-C₁₀ hydroxyalkyl.

In some embodiments, R⁴⁴ is —OH, —NH₂, C₁-C₁₀ hydroxyalkyl, or —NH—C₁-C₁₀ alkyl. In some embodiments, R⁴⁴ is hydrogen.

In some embodiments, R⁴⁵ is optionally substituted C₁-C₁₀ alkyl. In some embodiments, R⁴⁵ is methyl. In some embodiments, R⁴⁵ is hydrogen.

In some embodiments, p₁₀ is 3 or 4. In some embodiments, p₁₀ is 2. In some embodiments, p₁₀ is 1.

In some embodiments, the second terminus comprises Formula (11-A), or a pharmaceutically acceptable salt thereof:

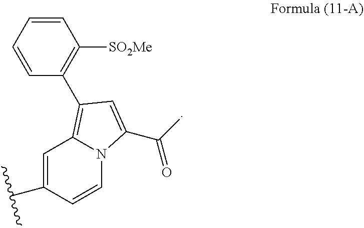

In some embodiments, the second terminus is selected from the group consisting of:

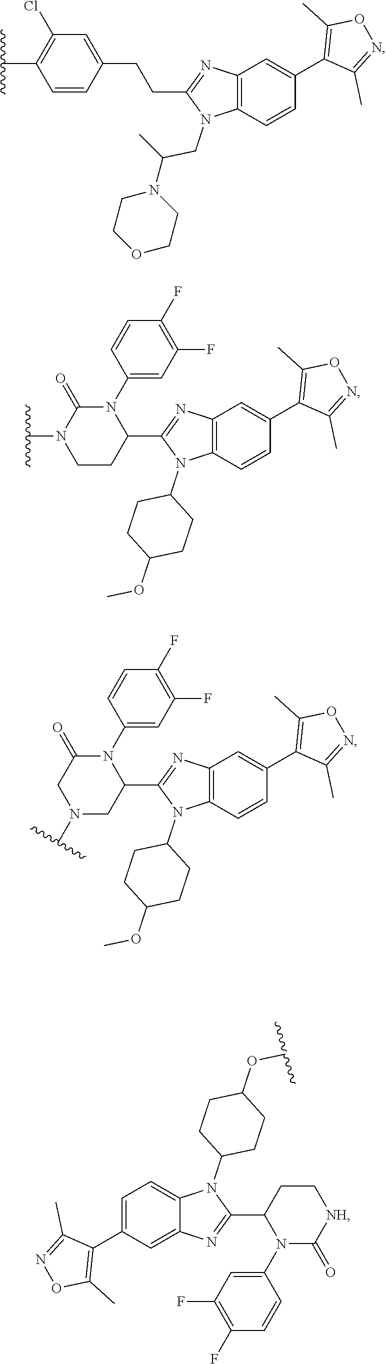

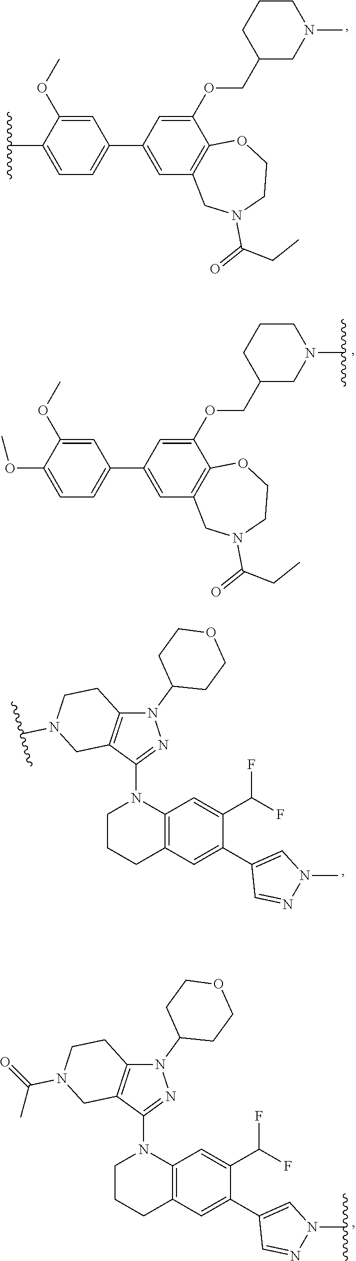

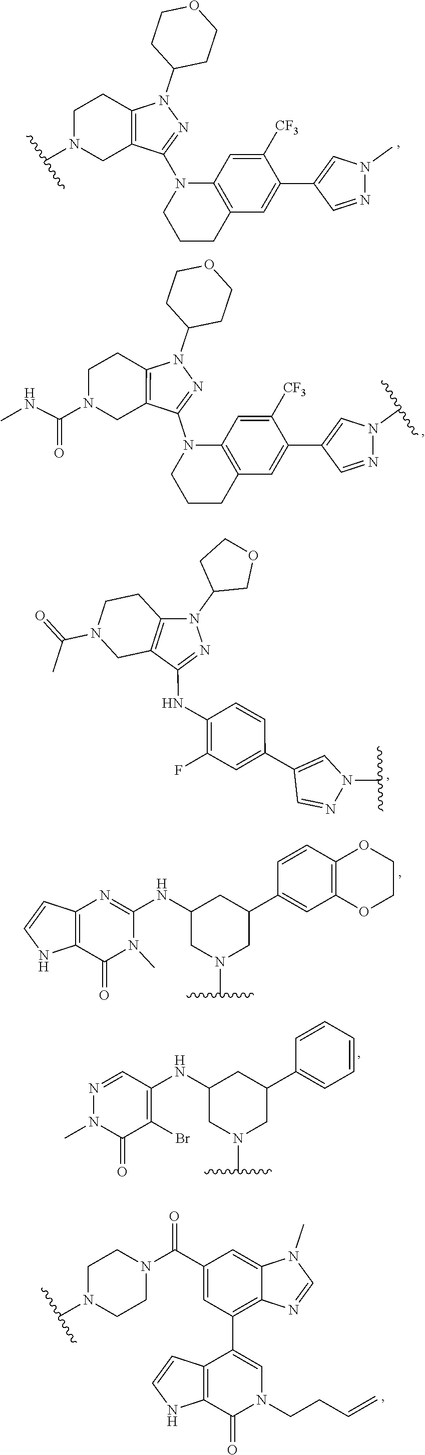

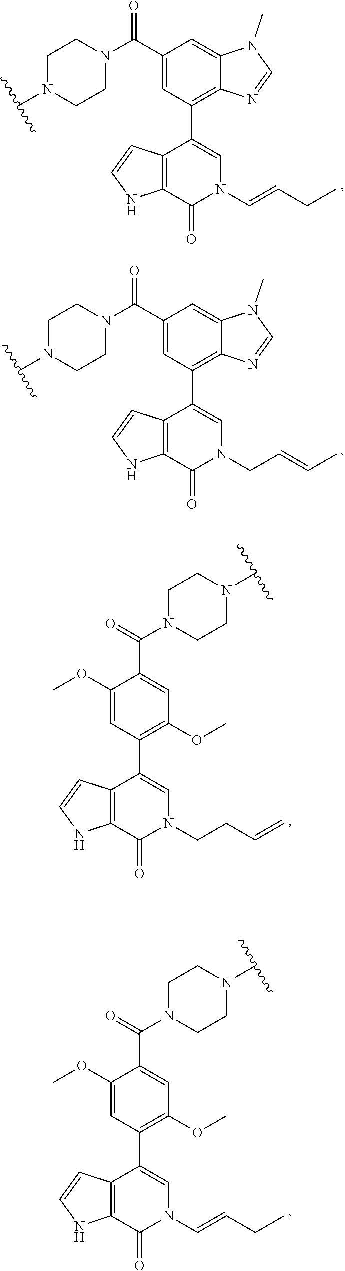

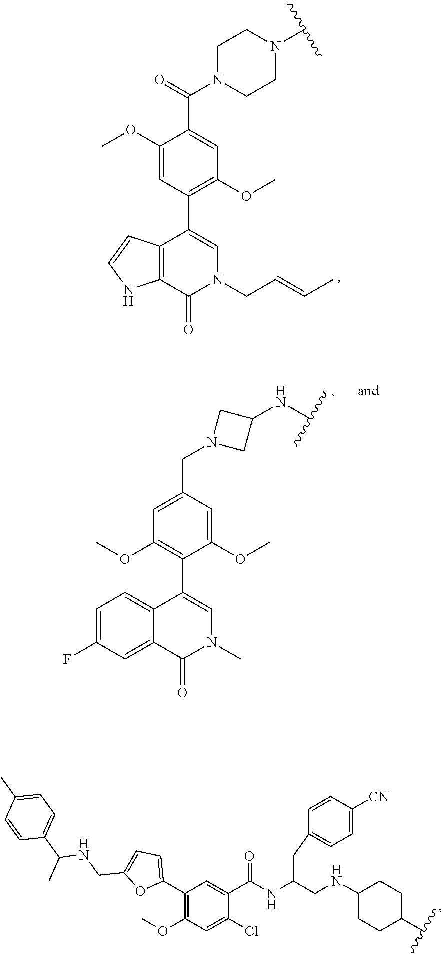 [SMILES: *N1CCN(C(=O)c2cc(OC)c(-c3cn(C/C=C/C)c(=O)c4[nH]ccc34)cc2OC)CC1 ; *NC1CN(Cc2cc(OC)c(-c3cn(C)c(=O)c4cc(F)ccc34)c(OC)c2)C1 ; *C1CCC(NCC(Cc2ccc(C#N)cc2)NC(=O)c2cc(-c3ccc(CNC(C)c4ccc(C)cc4)o3)c(OC)cc2Cl)CC1]

and pharmaceutically acceptable salts thereof.

In some embodiments, the second terminus is selected from a moiety described in Table 2.

TABLE 2
Exemplary bromodomain binding moieties.

| Structure | Binder |
|---|---|
| 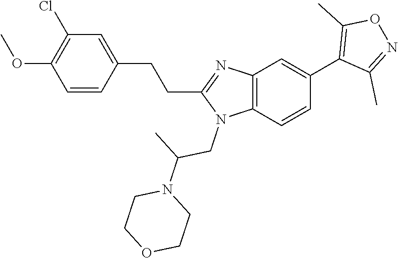 [SMILES: COc1ccc(CCc2nc3cc(-c4c(C)noc4C)ccc3n2CC(C)N2CCOCC2)cc1Cl] | CBP/P300 |
| 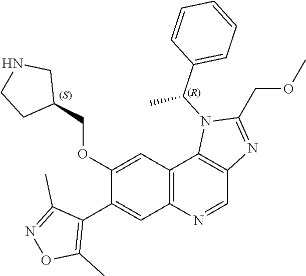 | BET(BD1) |
| 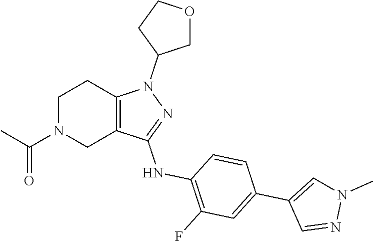 [SMILES: CC(=O)N1CCc2c(c(Nc3ccc(-c4cnn(C)c4)cc3F)nn2C2CCOC2)C1] | PCAF |
| 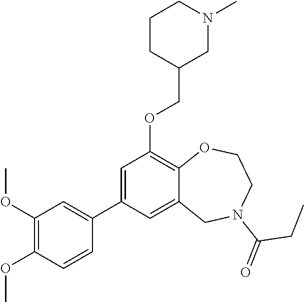 [SMILES: CCC(=O)N1CCOc2c(cc(-c3ccc(OC)c(OC)c3)cc2OCC2CCCN(C)C2)C1] | CBP/P300 |
| 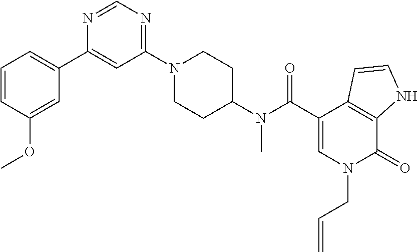 [SMILES: C=CCn1cc(C(=O)N(C)C2CCN(c3cc(-c4cccc(OC)c4)ncn3)CC2)c2cc[nH]c2c1=O] | CBP/P300 |
| 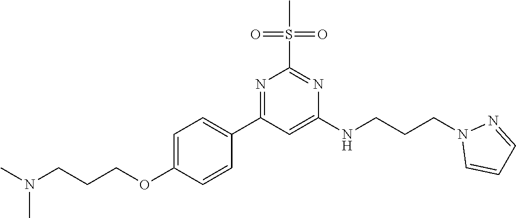 [SMILES: CN(C)CCCOc1ccc(-c2cc(NCCCn3cccn3)nc(S(C)(=O)=O)n2)cc1] | CECR2 |
| 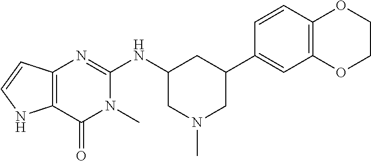 [SMILES: CN1CC(Nc2nc3cc[nH]c3c(=O)n2C)CC(c2ccc3c(c2)OCCO3)C1] | BPTF |
| 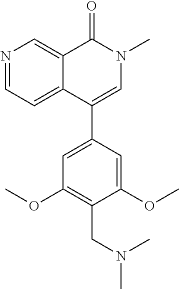 [SMILES: COc1cc(-c2cn(C)c(=O)c3cnccc23)cc(OC)c1CN(C)C] | PCAF |
| 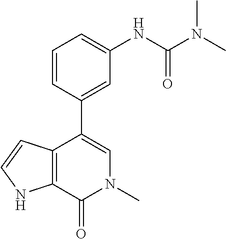 [SMILES: CN(C)C(=O)Nc1cccc(-c2cn(C)c(=O)c3[nH]ccc23)c1] | BRD7/9 |
| 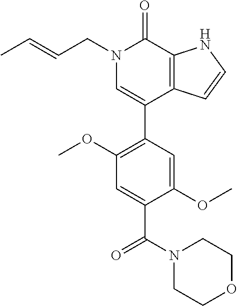 [SMILES: C/C=C/Cn1cc(-c2cc(OC)c(C(=O)N3CCOCC3)cc2OC)c2cc[nH]c2c1=O] | TAF1 |
| 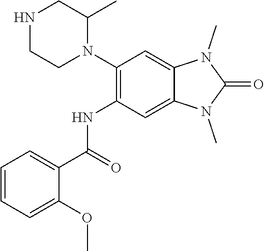 [SMILES: COc1ccccc1C(=O)Nc1cc2c(cc1N1CCNCC1C)n(C)c(=O)n2C] | BRD7/9 |
| 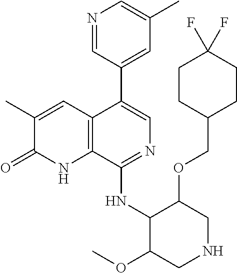 [SMILES: COC1CNCC(OCC2CCC(F)(F)CC2)C1Nc1ncc(-c2cncc(C)c2)c2cc(C)c(=O)[nH]c12] | BRPF |
| 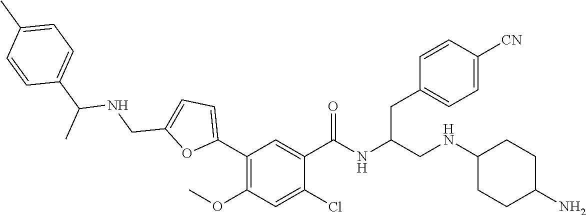 [SMILES: COc1cc(Cl)c(C(=O)NC(CNC2CCC(N)CC2)Cc2ccc(C#N)cc2)cc1-c1ccc(CNC(C)c2ccc(C)cc2)o1] | ATAD2/ATAD2B |
| 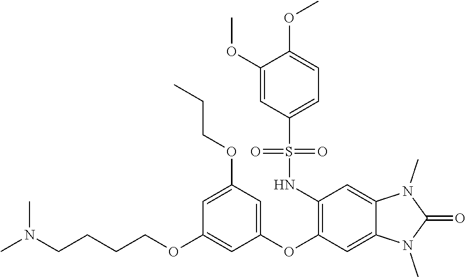 [SMILES: CCCOc1cc(OCCCCN(C)C)cc(Oc2cc3c(cc2NS(=O)(=O)c2ccc(OC)c(OC)c2)n(C)c(=O)n3C)c1] | TRIM24 |
| 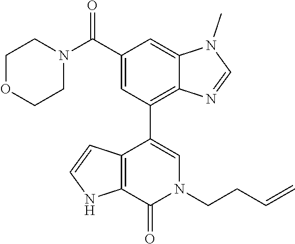 [SMILES: C=CCCn1cc(-c2cc(C(=O)N3CCOCC3)cc3c2ncn3C)c2cc[nH]c2c1=O] | TAF1 |

In some embodiments, the second terminus is not a bromodomain 4 (BRD4) ligand.

In some embodiments, the second terminus does not have a triazolodiazepine structure. In some embodiments, the second terminus does not comprise JQ1.

### Linker—Oligomeric Backbone

The oligomeric backbone contains a linker that connects the first terminus and the second terminus and brings the regulatory molecule in proximity to the target gene to modulate gene expression.

The length of the oligomeric backbone and/or linker depends on the type of regulatory protein and also the target gene. In some embodiments, the oligomeric backbone has a length of less than about 50 Angstroms. In some embodiments, the oligomeric backbone has a length of about 20 to 30 Angstroms.

In some embodiments, the oligomeric backbone comprises between 5 and 50 chain atoms.

In some embodiments, the oligomeric backbone comprises a multimer having 2 to 50 spacing moieties, wherein
each spacing moiety is independently selected from the group consisting of —((CR^{3a}R^{3b})ₓ—O)_{y}—, —((CR^{3a}R^{3b})ₓ—NR^{4a})_{y}—, —((CR^{3a}R^{3b})ₓ—CH═CH—(CR^{3a}R^{3b})ₓO)_{y}—, optionally substituted C₁-C₁₂ alkyl, optionally substituted C₂-C₁₀ alkenyl, optionally substituted C₂-C₁₀ alkynyl, optionally substituted C₆-C₁₀ arylene, optionally substituted C₃-C₇ cycloalkylene, optionally substituted 5- to 10-membered heteroarylene, optionally substituted 4- to 10-membered heterocycloalkylene, amino acid residue, —O—, —C(O)NR^{4a}_, —NR^{4a}C(O)—, —C(O)—, —NR^{1a}—, —C(O)O—, —S—, —S(O)—, —S(O)₂—, —S(O)₂NR^{4a}—, —NR^{4a}S(O)₂—, and —P(O)OH—, and any combinations thereof; wherein
each x is independently 2-4;
each y is independently 1-10;
each R^{1a} is independently a hydrogen or optionally substituted C₁-C₆ alkyl;
each R^{3a} and R^{3b} is independently selected from hydrogen, optionally substituted alkyl, optionally substituted alkenyl, optionally substituted alkynyl, optionally substituted alkoxy, optionally substituted amino, carboxyl, carboxyl ester, acyl, acyloxy, acyl amino, amino acyl, optionally substituted alkylamide, sulfonyl, optionally substituted thioalkoxy, optionally substituted aryl, optionally substituted heteroaryl, optionally substituted cycloalkyl, and optionally substituted heterocyclyl; and each R^{4a} is independently a hydrogen or an optionally substituted C₁-C₆ alkyl.

In some embodiments, the oligomeric backbone comprises -(T¹-V¹)ₐ-(T²-V²)_{b}-(T³-V³)_{c}-(T⁴-V⁴)_{d}-(T⁵-V⁵)ₑ-,
wherein a, b, c, d and e are each independently 0 or 1, and where the sum of a, b, c, d and e is 1 to 5;
T¹, T², T³, T⁴ and T are each independently selected from an optionally substituted C₁-C₁₂ alkylene, optionally substituted alkenylene, optionally substituted alkynylene, (EA)_{w}, (EDA)ₘ, (PEG)ₙ, (modified PEG)ₙ, (AA)ₚ, —(CR^{2a}OH)ₕ—, optionally substituted C₆-C₁₀ arylene, optionally substituted C₃-C₇ cycloalkylene, optionally substituted 5- to 10-membered heteroarylene, optionally substituted 4- to 10-membered heterocycloalkylene, an acetal group, a disulfide, a hydrazine, a carbohydrate, a beta-lactam, and an ester,
(a) w is an integer from 1 to 20;
(b) m is an integer from 1 to 20;
(c) n is an integer from 1 to 30;
(d) p is an integer from 1 to 20;
(e) h is an integer from 1 to 12;
(f) EA has the following structure

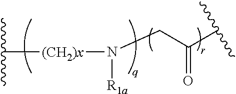

(g) EDA has the following structure:

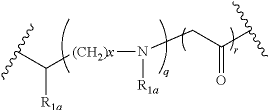

wherein each q is independently an integer from 1 to 6, each x is independently an integer from 1 to 4, and each r is independently 0 or 1;
(h) (PEG)ₙ has the structure of —(CR^{2a}R^{2b}—CR^{2a}R^{2b}—O)—CR^{2a}R^{2b};
(i) (modified PEG)ₙ has the structure of replacing at least one —(CR^{2a}R^{2b}—CR^{2a}R^{2b}—O)— in (PEG)ₙ with —(CH₂—CR^{2a}=CR^{2a}—CH₂—O)— or —(CR^{2a}R^{2b}—CR^{2a}R^{2b}—S)—;
(j) AA is an amino acid residue;
(k) V¹, V², V³, V⁴ and V⁵ are each independently selected from the group consisting of a bond, C(O)—, —NR^{1a}—, —C(O)NR^{1a}—, —NR^{1a}C(O)—, —CONR^{1a}—C₁-C₄ alkyl-, —NR^{1a}C(O)—C₁-C₄ alkyl-, —C(O)O—, —OC(O)—, —O—, —S—, —S(O)—, —S(O)₂—, —S(O)₂NR^{1a}—, —NR^{1a}S(O)₂— and —P(O)OH—;
each R^{1a} is independently hydrogen or and optionally substituted C₁-C₆ alkyl; and
each R^{2a} and R^{2b} is independently selected from hydrogen, alkyl, substituted alkyl, alkenyl, substituted alkenyl, alkynyl, substituted alkynyl, halogen, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In some embodiments, the a, b, c, d and e are each independently 0 or 1, where the sum of a, b, c, d and e is 1. In some embodiments, the a, b, c, d and e are each independently 0 or 1, where the sum of a, b, c, d and e is 2. In some embodiments, the a, b, c, d and e are each independently 0 or 1, where the sum of a, b, c, d and e is 3. In some embodiments, the a, b, c, d and e are each independently 0 or 1, where the sum of a, b, c, d and e is 4. In some embodiments, the a, b, c, d and e are each independently 0 or 1, where the sum of a, b, c, d and e is 5.

In some embodiments, n is 3-9. In some embodiments, n is 4-8. In some embodiments, n is 5 or 6.

In some embodiments, T¹, T², T³, and T⁴, and T⁵ are each independently selected from (C₁-C₁₂)alkyl, substituted (C₁-C₁₂)alkyl, (EA)_{w}, (EDA)ₘ, (PEG)ₙ, (modified PEG)ₙ, (AA)ₚ, —(CR^{2a}OH)ₕ—, phenyl, substituted phenyl, piperidin-4-amino (P4A), para-amino-benzyloxycarbonyl (PABC), meta-amino-benzyloxycarbonyl (MABC), para-amino-benzyloxy (PABO), meta-amino-benzyloxy (MABO), para-aminobenzyl, an acetal group, a disulfide, a hydrazine, a carbohydrate, a beta-lactam, an ester, (AA)ₚ-MABC-(AA)ₚ, (AA)ₚ-MABO-(AA)ₚ, (AA)ₚ-PABO-(AA)ₚ and (AA)ₚ-PABC-(AA)ₚ, In some embodiments, piperidin-4-amino (P4A) is

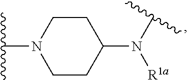

wherein R^{1a} is H or C₁₋₆alkyl.

In some embodiments, T¹, T², T³, T⁴ and T⁵ are each independently selected from (C₁-C₁₂)alkyl, substituted (C₁-C₁₂)alkyl, (EA)_{w}, (EDA)ₘ, (PEG)ₙ, (modified PEG)ₙ, (AA)ₚ, —(CR^{2a}OH)ₕ—, optionally substituted (C₆-C₁₀) arylene, 4-10 membered heterocycloalkene, optionally substituted 5-10 membered heteroarylene. In some embodiments, EA has the following structure:

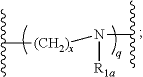

and
EDA has the following structure:

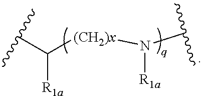

In some embodiments, x is 2-3 and q is 1-3 for EA and EDA. In some embodiments, R^{1a} is H or C₁-C₆ alkyl.

In some embodiments, T⁴ or T⁵ is an optionally substituted C₆-C₁₀ arylene.

In some embodiments, T⁴ or T⁵ is phenylene or substituted phenylene. In some embodiments, T⁴ or T⁵ is phenylene or phenylene substituted with 1-3 substituents selected from C₁-C₆ alkyl, halogen, OH or amine. In some embodiments, T⁴ or T⁵ is 5 to 10-membered heteroarylene or substituted heteroarylene. In some embodiments, T⁴ or T⁵ is 4 to 10-membered heterocyclene or substituted heterocyclylene. In some embodiments, T⁴ or T⁵ is heteroarylene or heterocyclene optionally substituted with 1-3 substituents selected from C₁-C₆alkyl, halogen, OH or amine.

In some embodiments, T¹, T², T³, T⁴ and T⁵ and V¹, V², V¹, V⁴ and V⁵ are selected from the following Table 3.

TABLE 3
Representative linker units.

| T¹ | V¹ | T² | V² | T³ | V³ | T⁴ | V⁴ | T⁵ | V⁵ |
|---|---|---|---|---|---|---|---|---|---|
| C₁-C₁₂ | CONR^{1a} | (EA)_{w} | CO | (PEG)ₙ | NR^{1a}CO | — | — | — | — |
| alkylene | | | | | | | | | |
| C₁-C₁₂ | CONR^{1a} | (EA)_{w} | CO | (PEG)ₙ | O | arylene | NR^{1a}CO | — | — |
| alkylene | | | | | | | | | |
| C₁-C₁₂ | CONR^{1a} | (EA)_{w} | CO | (PEG)ₙ | O | Subst. | NR^{1a}CO | — | — |
| alkylene | | | | | | arylene | | | |
| C₁-C₁₂ | CONR^{1a} | (EA)_{w} | CO | (PEG)ₙ | O | NR^{1a}CO | C₁-C₁₂ | Subst. | NR^{1a}CO |
| alkylene | | | | | | | alkyl | arylene | |
| C₁-C₁₂ | CONR^{1a} | (EA)_{w} | CO | C₁-C₁₂ | NR^{1a}CO- | Subst. | NR^{1a} | — | — |
| alkylene | | | | alkyl | C₁-C₄ | arylene | | | |
| | | | | | alkyl | | | | |
| C₁-C₁₂ | CONR^{1a} | (EA)_{w} | CO | (PEG)ₙ | O | Subst. | — | — | — |
| alkylene | | | | | | arylene | | | |
| (PEG)ₙ | CONR^{1a-} | — | — | — | — | — | — | — | — |
| | C₁-C₄ | | | | | | | | |
| | alkyl | | | | | | | | |
| (EA)_{w} | CO | C₁-C₁₂ | CONR^{1a-} | — | — | — | — | — | — |
| | | alkyl | C₁-C₄ | | | | | | |
| | | | alkyl | | | | | | |
| C₁-C₁₂ | CONR^{1a} | (EA)_{w} | CO | (PEG)ₙ | NR^{1a}CO- | — | — | — | — |
| alkylene | | | | | C₁-C₄ | | | | |
| | | | | | alkyl | | | | |
| (EA)_{w} | CO | (PEG)ₙ | O | phenyl | NR^{1a}CO- | — | — | — | — |
| | | | | | C₁-C₄ | | | | |
| | | | | | alkyl | | | | |
| (C₁-C₁₂) | CONR^{1a} | (PEG)ₙ | CO | — | — | — | — | — | — |
| alkylene | | | | | | | | | |
| (C₁-C₁₂) | CONR^{1a} | (EA)_{w} | CO | modifd. | O | arylene | NR^{1a}CO | — | — |
| alkylene | | | | (PEG)ₙ | | | | | |

In some embodiments, the oligomeric backbone comprises —N(R^{1a})(CH₂)ₓN(R^{1b})(CH₂)ₓN—, wherein R^{1a} and R^{1b} are each independently selected from hydrogen or optionally substituted C₁-C₆ alkyl; and each x is independently an integer in the range of 1-6.

In some embodiments, the oligomeric backbone comprises —NR^{1a}(CH₂CH₂O)_{y}(CH₂)ₓ— or —NR^{1a}—(CH₂)_{q}—C(O)NR^{1a}(CH₂CH₂O)_{y}(CH₂)ₓ—, wherein q is 2-10, x is 1-6, y is 1-50, and each R^{1a} is independently hydrogen or an optionally substituted C₁-C₆ alkyl. In some embodiments, the oligomeric backbone comprises —NR^{1a}(CH₂CH₂O)_{y}(CH₂)ₓ—. In some embodiments, the oligomeric backbone comprises —NR^{1a}—(CH₂)_{q}—C(O)NR^{1a}(CH₂CH₂O)_{y}(CH₂)ₓ—. In some embodiments, the oligomeric backbone comprises —NH(CH₂CH₂O)_{y}(CH₂)ₓ—. In some embodiments, the oligomeric backbone comprises —NH—(CH₂)_{q}—C(O)NH(CH₂CH₂O)_{y}(CH₂)ₓ—.

In some embodiments, the oligomeric backbone comprises —(CH₂CH₂—O)_{y}—, —(CH₂CH₂—O)_{y}—(CH₂CH₂)—NH—, —NH—(CH₂CH₂—O)_{y}—, —NH—(CH₂CH₂—O)_{y}—(CH₂CH₂)—NH—, —(CH₂CH₂—O)_{y}—(CH₂CH₂)—NHC(O)—, or —NH—(CH₂CH₂—O)_{y}—(CH₂CH₂)—NHC(O)—, wherein y is 1-50. In some embodiments, the oligomeric backbone comprises —NH—(CH₂CH₂—O)_{y}— or —NH—(CH₂CH₂—O)_{y}—(CH₂CH₂)—NH—. In some embodiments, the oligomeric backbone comprises —NH—(CH₂CH₂—O)_{y}—. In some embodiments, the oligomeric backbone comprises —NH—(CH₂CH₂—O)_{y}—(CH₂CH₂)—NH—. In some embodiments, y is 1-40, 1-35, 1-30, 1-25, 1-20, 1-18, 1-16, 1-14, 1-12, 1-10, 1-9, 1-8, 1-7, 1-6, 1-5, 1-4, 1-3, or 1-2. In some embodiments, y is 1-20. In some embodiments, y is 1-18. In some embodiments, y is 1-16. In some embodiments, y is 1-14. In some embodiments, y is 1-12. In some embodiments, y is 1-10. In some embodiments, y is 1-8. In some embodiments, y is 1-6. In some embodiments, y is 1-4.

In some embodiments, the oligomeric backbone comprises polyethylene glycol (“PEG”). In some embodiments, the oligomeric backbone comprises 1-20 PEG units. In some embodiments, the oligomeric backbone comprises 1, 2, 3, 4, 5, 6, 7, 8, 9, 10, 11, 12, 13, 14, 15, 16, 17, 18, 19, or 20 PEG units.

In some embodiments, the oligomeric backbone comprises —(CH₂—C(O)N(R^{1a}—(CH₂)_{q}—N(R^{1b})—(CH₂)_{q}—N(R^{1a})C(O)—(CH₂)ₓ—C(O)N(R^{1a})-A²-, —(CH₂)ₓ—C(O)N(R^{1a})—(CH₂CH₂O)_{y}(CH₂)ₓ—C(O)N(R^{1a})-A²-, —C(O)N(R^{1a})—(CH₂)_{q}—N(R^{1b})—(CH₂)_{q}—N(R^{1a})C(O)—(CH₂)ₓ-A²-, —(CH₂)ₓ—O—(CH₂CH₂O)_{y}—(CH₂)ₓ—N(R^{1a})C(O)—(CH₂)ₓ-A²-, or —N(R^{1a})C(O)—(CH₂)—C(O)N(R^{1a})—(CH₂)ₓ—O(CH₂CH₂O)_{y}(CH₂)ₓ-A²-; wherein R^{1a} is hydrogen or optionally substituted C₁-C₃ alkyl; R^{1b} is hydrogen; each x and y are independently an integer from 1 to 10; each q is independently an integer from 2 to 10; and each A² is independently selected from a bond, an optionally substituted C₁-C₁₂ alkyl, an optionally substituted C₆-C₁₀ arylene, optionally substituted C₃-C₇ cycloalkylene, optionally substituted 5 to 10-membered heteroarylene, and optionally substituted 4 to 10-membered heterocycloalkylene.

In some embodiments, the oligomeric backbone comprises —(CH₂CH—O)ₓ— or —(CH₂CH₂—O)ₓ-A²-(CH₂CH₂—O)ₓ—, wherein A² is an optionally substituted 4- to 10-membered heterocycloalkylene or spirocyclene, and each x, x₁, and x₂ is independently an integer from 1-15.

In some embodiments, A² is selected from

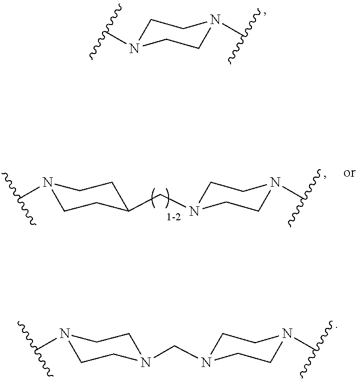

In some embodiments, A² is

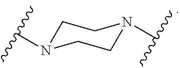 [SMILES: *N1CCN(*)CC1]

In some embodiments, A² is

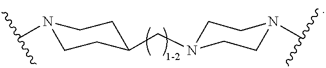

In some embodiments, A is

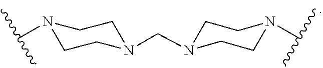 [SMILES: *N1CCN(CN2CCN(*)CC2)CC1]

In some embodiments, A² comprises a moiety having the structure:

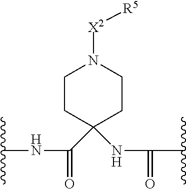

wherein,
X² is absent or —C(O)—; and
R⁵ is an optionally substituted C₁-C₅₀ alkyl or optionally substituted C₁-C₅₀ heteroalkyl.

In some embodiments, X² is —C(O)—. In some embodiments, X² is absent.

In some embodiments, R⁵ is C₁-C₅₀ alkyl. In some embodiments, R⁵ is C₁-C₄₀ alkyl. In some embodiments, R⁵ is C₁-C₃₀ alkyl. In some embodiments, R⁵ is C₁-C₂₀ alkyl. In some embodiments, R⁵ is C₁-C₁₀ alkyl. In some embodiments, R⁵ is C₁-C₅₀ heteroalkyl. In some embodiments. R⁵ is C₁-C₄₀ heteroalkyl. In some embodiments, R⁵ is C₁-C₃₀ heteroalkyl. In some embodiments, R⁵ is C₁-C₂₀ heteroalkyl. In some embodiments, R⁵ is C₁-C₁₀ heteroalkyl. In some embodiments, the heteroalkyl is polyethylene glycol (PEG).

In some embodiments, the oligomeric backbone or linker is joined with the first terminus with a group selected from —C(O)—, —NR^{1a}—, —C(O)NR^{1a}—, —NR^{1a}C(O)—, —C(O)NR^{1a}—C₁-C₄alkyl-, —NR^{1a}C(O)—Cr C₄ alkyl-, —C(O)O—, —OC(O)—, —O—, —S—, —SO—, —SO₂—, —SO₂NR^{1a}—, —NR^{1a}SO₂—, —P(O)OH—, —((CH₂)ₓ—O)—, —((CH₂)_{y}—NR^{1a})—, optionally substituted C₁-C₁₂ alkylene, optionally substituted C₁-C₁₀ alkenylene, optionally substituted C₂-C₁₀ alkynylene, optionally substituted C₆-C₁₀ arylene, optionally substituted C₃-C₇ cycloalkylene, optionally substituted 5 to 10-membered heteroarylene, and optionally substituted 4 to 10-membered heterocycloalkylene, wherein each x is independently 1-4, each y is independently 1-4, and each R^{1a} is independently a hydrogen or optionally substituted C₁-C₆ alkyl.

In some embodiments, the oligomeric backbone or linker is joined with the first terminus with a group selected from —C(O)—, —NR^{1a}—, C₁-C₂ alkyl, —C(O)NR^{1a}—, and —NR^{1a}C(O)—; wherein each R^{1a} is independently a hydrogen or optionally substituted C₁-C₆ alkyl. In some embodiments, the oligomeric backbone or linker is joined with the first terminus with —NR^{1a}— or —O—. In some embodiments, the oligomeric backbone or linker is joined with the first terminus with —NR^{1a}—. In some embodiments, the oligomeric backbone or linker is joined with the first terminus with —NH—. In some embodiments, the oligomeric backbone or linker is joined with the first terminus with —O—.

In some embodiments, the oligomeric backbone or linker is joined with the second terminus with a group selected from —C(O)—, —NR^{1a}—, —C(O)NR^{1a}—, —NR^{1a}C(O)—, —C(O)NR^{1a}—C₁-C₄alkyl-, —NR^{1a}C(O)—C₁-C₄ alkyl-, —C(O)O—, —OC(O)—, —O—, —S—, —SO—, —SO₂—, —SO₂NR^{1a}—, —NR^{1a}SO₂—, —P(O)OH—, —((CH₂)ᵣO)—, —((CH₂)_{y}—NR^{1a})—, optionally substituted C₁-C₁₂ alkylene, optionally substituted C₂-C₁₀ alkenylene, optionally substituted C₂-C₁₀ alkynylene, optionally substituted C₆-C₁₀ arylene, optionally substituted C₃-C₇ cycloalkylene, optionally substituted 5 to 10-membered heteroarylene, and optionally substituted 4 to 10-membered heterocycloalkylene, wherein each x is independently 1-4, each y is independently 1-4, and each R^{1a} is independently a hydrogen or optionally substituted C₁-C₆ alkyl.

In some embodiments, the oligomeric backbone or linker is joined with the second terminus with a group selected from —C(O)—, —NR^{1a}—, C₁-C₁₂ alkyl, —C(O)NR^{1a}—, and —NR^{1a}C(O)—; wherein each R^{1a} is independently a hydrogen or optionally substituted C₁-C₆ alkyl. In some embodiments, the oligomeric backbone or linker is joined with the second terminus with —NR^{1a}— or —O—. In some embodiments, the oligomeric backbone or linker is joined with the second terminus with —NR^{1a}—. In some embodiments, the oligomeric backbone or linker is joined with the second terminus with —NH—. In some embodiments, the oligomeric backbone or linker is joined with the second terminus with —O—.

In some embodiments, the oligomeric backbone or linker is joined with the second terminus with a group selected from optionally substituted 4 to 10-membered heterocycloalkylene.

In some embodiments, the oligomeric backbone or linker is joined with the second terminus with a moiety comprising a structure of Formula (C-1):

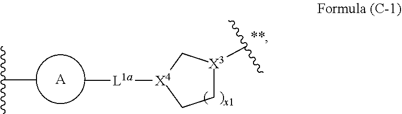

wherein;
ring A is absent, arylene, or heterocycloalkylene;
L^{1a} is absent, optionally substituted alkylene, or optionally substituted alkynylene;
each X³ and X⁴ is independently CH or N;
x₁ is 0-3; and
** denotes attachment to the second terminus.

In some embodiments, ring A is absent. In some embodiments, ring A is C₄-C₇ heterocycloalkylene.

In some embodiments, X³ is N. In some embodiments, X³ is CH.

In some embodiments, X⁴ is N. In some embodiments, X⁴ is CH.

In some embodiments, the oligomeric backbone or linker is joined with second terminus comprises a structure of Formula (C-2):

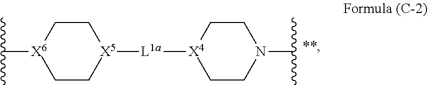

wherein; each X⁵ and X⁶ is independently N or CH.

In some embodiments, each of X⁴ and X⁵ is independently N or CH; and X⁶ is N.

In some embodiments, L^{1a} is absent.

In some embodiments, L^{1a} is —(CR^{1m}R^{1m})ₓ-(alkylene)₂-(CR^{1m}R^{1m})_{y}—; wherein x and y are each independently 0 or 1; and each R^{1m} is hydrogen or C₁-C₃ alkyl.

In some embodiments, L^{1a} is C₁-C₃ alkylene or C₁-C₃ alkynylene.

In some embodiments, L^{1a} is —CH₂—, —CH₂CH₂—, —C≡C—, or —C≡C—C≡C—. In some embodiments, L^{1a} is —CH₂— or —CH₂CH₂—. In some embodiments, L^{1a} is —C≡C—. In some embodiments, L¹ is —C≡C—C≡C—.

In some embodiments, the oligomeric backbone or linker is joined with the second terminus with a moiety comprising a structure of Formula (C-3):

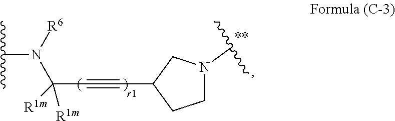

wherein;
x₁ is 0-3;
r₁ is 1-3;
R⁶ is an C₁-C₅₀ alkyl, C₁-C₅₀ heteroalkyl, —C(O)C₁-C₅₀ alkyl, or —C(O)C₁-C₅₀ heteroalkyl, wherein each alkyl and heteroalkyl is optionally substituted;
each R^{1m} is independently hydrogen or C₁-C₃ alkyl; and
** denotes attachment to the second terminus.

In some embodiments, R⁶ is an optionally substituted C₁-C₅₀ alkyl or optionally substituted C₁-C₅₀ heteroalkyl. In some embodiments, R⁶ is —C(O)C₁-C₅₀ alkyl or —C(O)C₁-C₅₀ heteroalkyl, wherein the alkyl and heteroalkyl is optionally substituted.

some embodiments, R⁶ is C₁-C₅₀ alkyl. In some embodiments, R²⁷ is C₁-C₄₀ alkyl. In some embodiments, R⁶ is C₁-C₃₀ alkyl. In some embodiments, R⁶ is C₁-C₂₀ alkyl. In some embodiments, R⁶ is C₁-C₁₀ alkyl. In some embodiments, R⁶ is C₁-C₅₀ heteroalkyl. In some embodiments. R⁶ is C₁-C₄₀ heteroalkyl. In some embodiments, R⁶ is C₁-C₃₀ heteroalkyl. In some embodiments, R⁶ is C₁-C₂₀ heteroalkyl. In some embodiments, R⁶ is C₁-C₁₀ heteroalkyl. In some embodiments, the heteroalkyl is polyethylene glycol (“PEG”).

In some embodiments, each R^{1m} is independently hydrogen. In some embodiments, R^{1m} is independently C₁-C₃ alkyl. In some embodiments, the C₁-C₃ alkyl is methyl, ethyl or propyl. In some embodiments, each R^{1m} is independently methyl.

In some embodiments, x₁ is 0, 1, or 2. In some embodiments, x₁ is 0. In some embodiments, x₁ is 1. In some embodiments, x₁ is 2.

In some embodiments, r₁ is 1 or 2. In some embodiments, r₁ is 1. In some embodiments, r₁ is 2.

In some embodiments, the oligomeric backbone is joined with the second terminus with a group selected from:

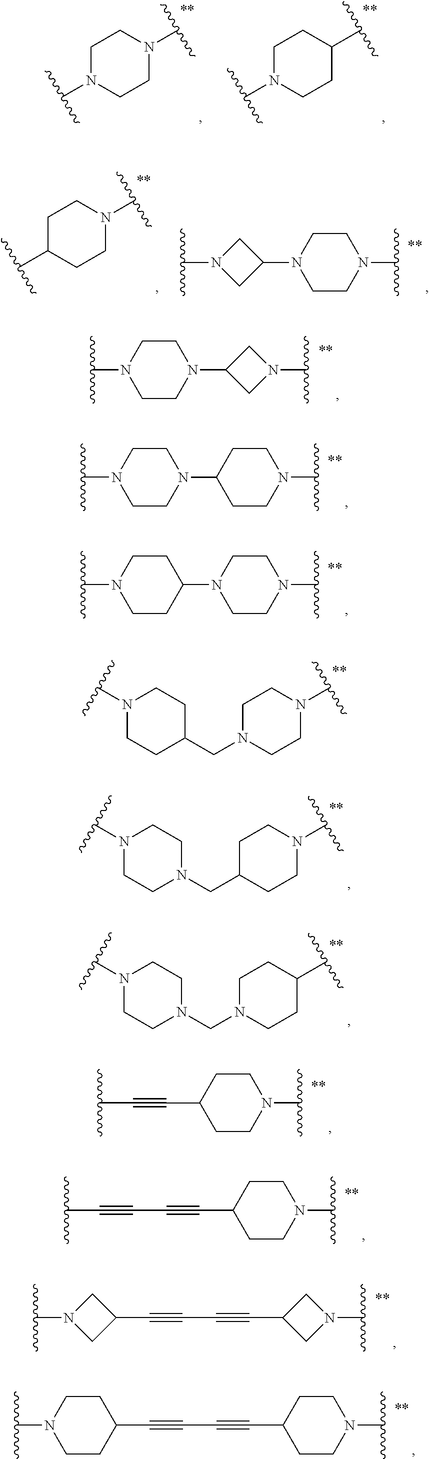

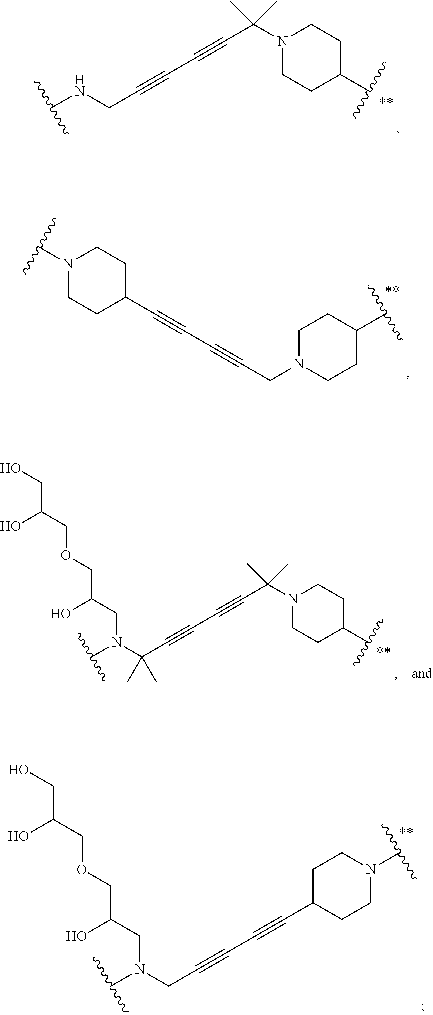 [SMILES: *NCC#CC#CC(C)(C)N1CCC(*)CC1 ; *C1CCN(CC#CC#CC2CCN(*)CC2)CC1 ; *C1CCN(C(C)(C)C#CC#CC(C)(C)N(*)CC(O)COCC(O)CO)CC1 ; *N1CCC(C#CC#CCN(*)CC(O)COCC(O)CO)CC1]

wherein ** denotes the connection to the first and/or the second terminus.

In some embodiments, the oligomeric backbone is joined with the second terminus with a group selected from:

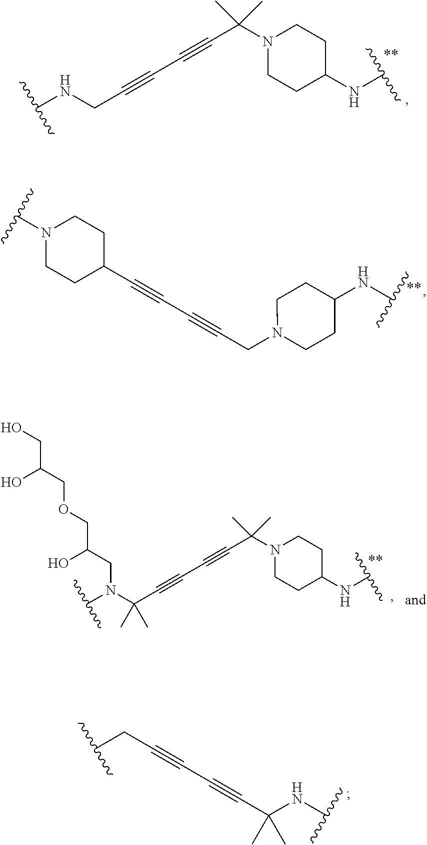 [SMILES: *NCC#CC#CC(C)(C)N1CCC(N*)CC1 ; *NC1CCN(CC#CC#CC2CCN(*)CC2)CC1 ; *NC1CCN(C(C)(C)C#CC#CC(C)(C)N(*)CC(O)COCC(O)CO)CC1 ; *CC#CC#CC(C)(C)N*]

and wherein ** denotes the connection to the first and/or the second terminus.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

In some embodiments, non-limiting examples of the compounds described herein are presented in Table 4 (next page).

| Cmp. | |
|---|---|
| No. | Structure |
| A−1 | 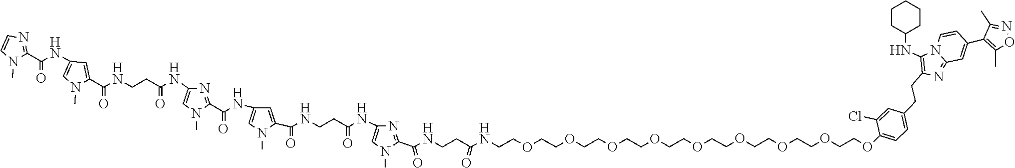 [SMILES: Cc1noc(C)c1-c1ccn2c(NC3CCCCC3)c(CCc3ccc(OCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)c(Cl)c3)nc2c1] |
| A−2 | 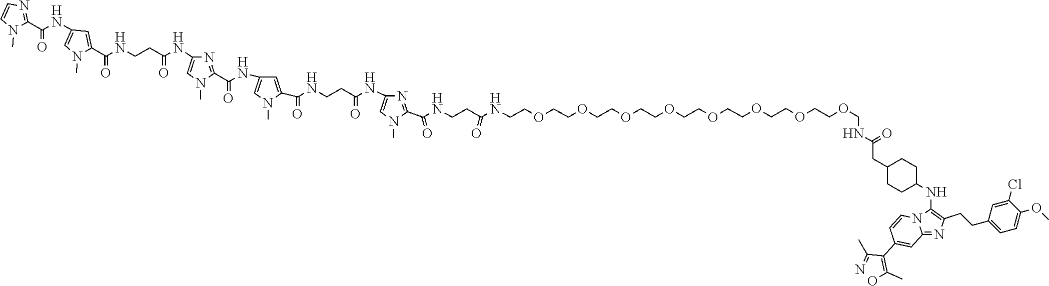 [SMILES: COc1ccc(CCc2nc3cc(-c4c(C)noc4C)ccn3c2NC2CCC(CC(=O)NCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC2)cc1Cl] |
| A−3 | 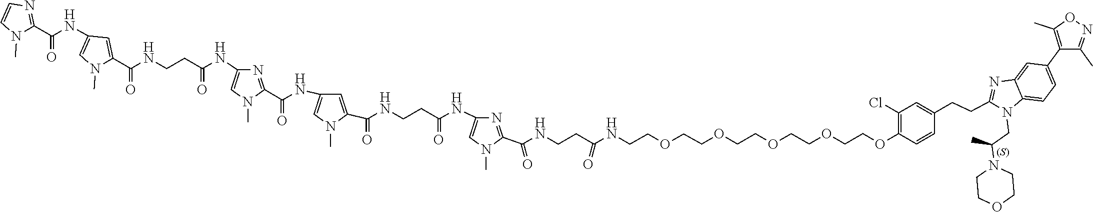 |
| A−4 | 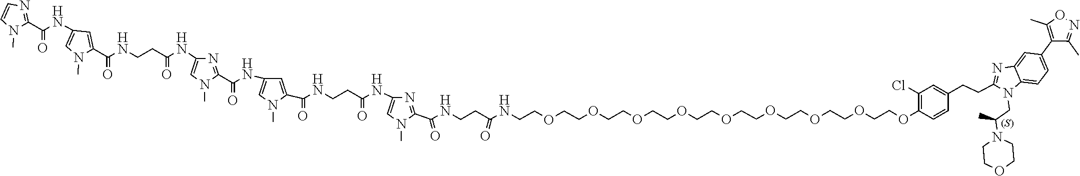 |
| A−5 | 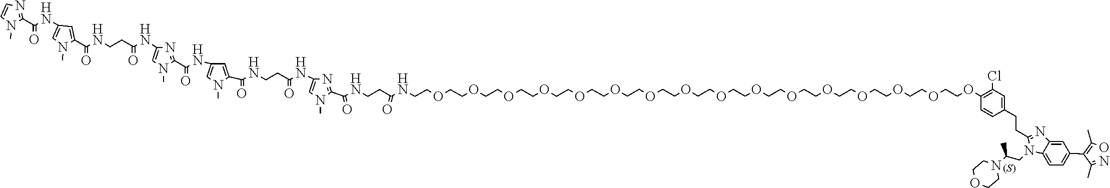 |
| A−6 | 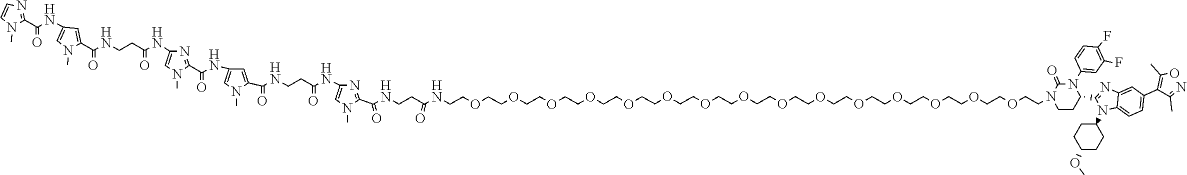 [SMILES: CO[C@H]1CC[C@H](n2c([C@@H]3CCN(CCOCCOCCOCCOCCOCCOCCOCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)C(=O)N3c3ccc(F)c(F)c3)nc3cc(-c4c(C)noc4C)ccc32)CC1] |
| A−7 | 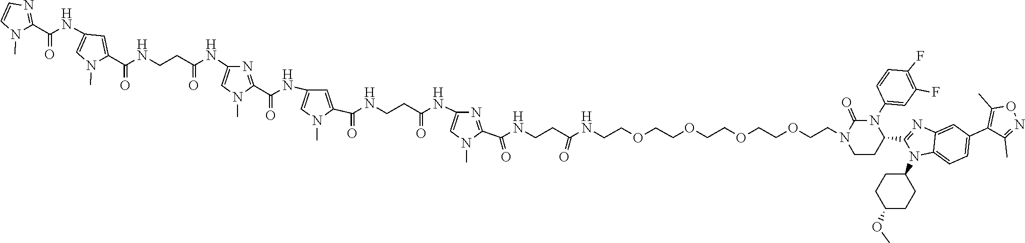 [SMILES: CO[C@H]1CC[C@H](n2c([C@@H]3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)C(=O)N3c3ccc(F)c(F)c3)nc3cc(-c4c(C)noc4C)ccc32)CC1] |
| A−8 | 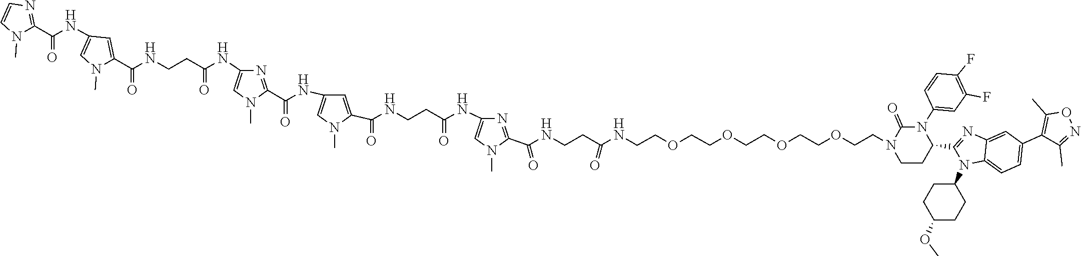 [SMILES: CO[C@H]1CC[C@H](n2c([C@@H]3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)C(=O)N3c3ccc(F)c(F)c3)nc3cc(-c4c(C)noc4C)ccc32)CC1] |
| A−9 | 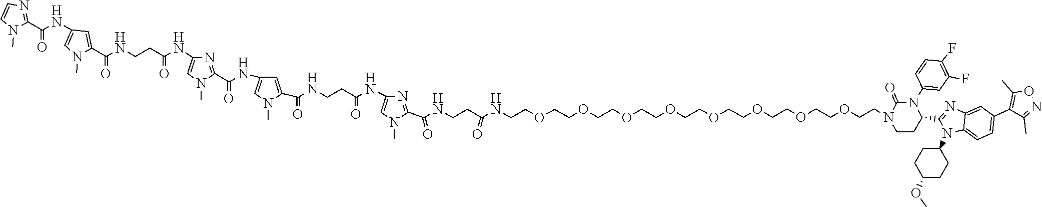 [SMILES: CO[C@H]1CC[C@H](n2c([C@@H]3CCN(CCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)C(=O)N3c3ccc(F)c(F)c3)nc3cc(-c4c(C)noc4C)ccc32)CC1] |
| A−10 | 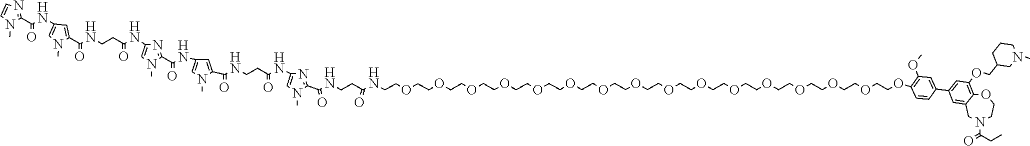 [SMILES: CCC(=O)N1CCOc2c(cc(-c3ccc(OCCOCCOCCOCCOCCOCCOCCOCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)c(OC)c3)cc2OCC2CCCN(C)C2)C1] |
| A−11 | 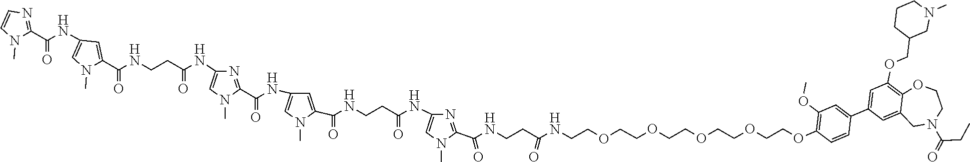 [SMILES: CCC(=O)N1CCOc2c(cc(-c3ccc(OCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)c(OC)c3)cc2OCC2CCCN(C)C2)C1] |
| A−12 | 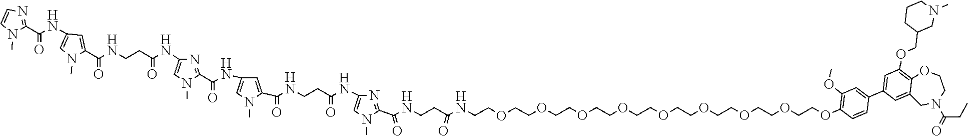 [SMILES: CCC(=O)N1CCOc2c(cc(-c3ccc(OCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)c(OC)c3)cc2OCC2CCCN(C)C2)C1] |
| A−13 | 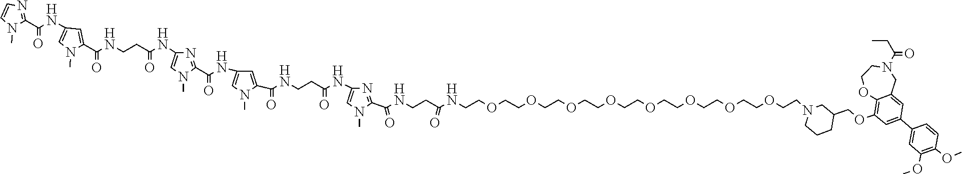 [SMILES: CCC(=O)N1CCOc2c(cc(-c3ccc(OC)c(OC)c3)cc2OCC2CCCN(CCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)C2)C1] |
| A−14 | 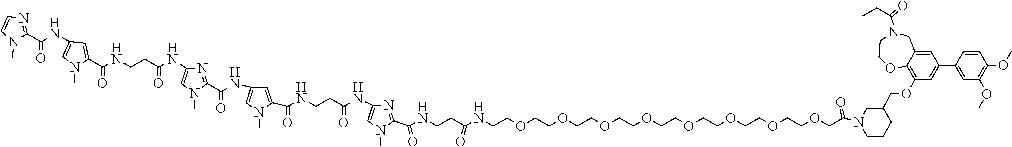 [SMILES: CCC(=O)N1CCOc2c(cc(-c3ccc(OC)c(OC)c3)cc2OCC2CCCN(C(=O)COCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)C2)C1] |
| A−15 | 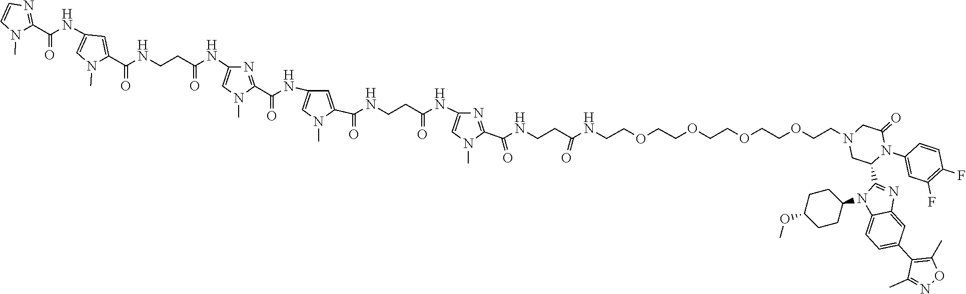 [SMILES: CO[C@H]1CC[C@H](n2c([C@@H]3CN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC(=O)N3c3ccc(F)c(F)c3)nc3cc(-c4c(C)noc4C)ccc32)CC1] |
| A−16 | 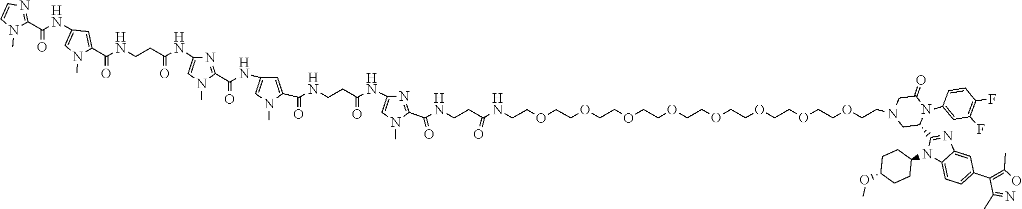 [SMILES: CO[C@H]1CC[C@H](n2c([C@@H]3CN(CCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC(=O)N3c3ccc(F)c(F)c3)nc3cc(-c4c(C)noc4C)ccc32)CC1] |
| A−17 | 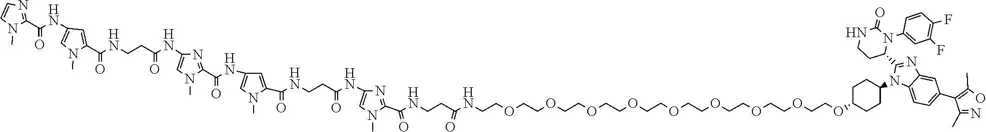 [SMILES: Cc1noc(C)c1-c1ccc2c(c1)nc([C@@H]1CCNC(=O)N1c1ccc(F)c(F)c1)n2[C@H]1CC[C@H](OCCOCCOCCOCCOCCOCCOCCOCCOCCNC(=O)CCNC(=O)c2nc(NC(=O)CCNC(=O)c3cc(NC(=O)c4nc(NC(=O)CCNC(=O)c5cc(NC(=O)c6nccn6C)cn5C)cn4C)cn3C)cn2C)CC1] |
| A−18 | 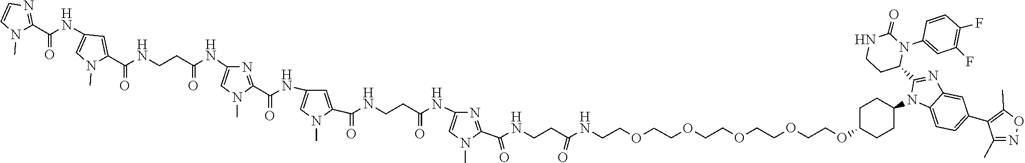 [SMILES: Cc1noc(C)c1-c1ccc2c(c1)nc([C@@H]1CCNC(=O)N1c1ccc(F)c(F)c1)n2[C@H]1CC[C@H](OCCOCCOCCOCCOCCNC(=O)CCNC(=O)c2nc(NC(=O)CCNC(=O)c3cc(NC(=O)c4nc(NC(=O)CCNC(=O)c5cc(NC(=O)c6nccn6C)cn5C)cn4C)cn3C)cn2C)CC1] |
| A−19 | 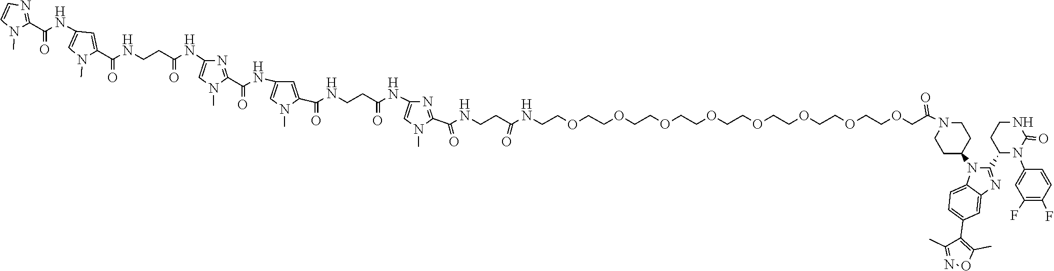 |
| A−20 | 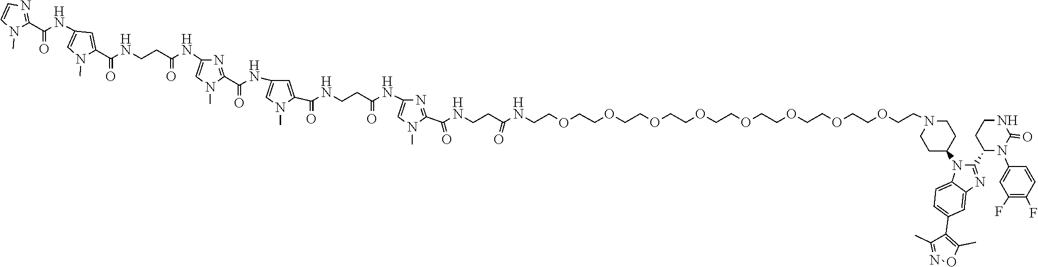 |
| A−21 | 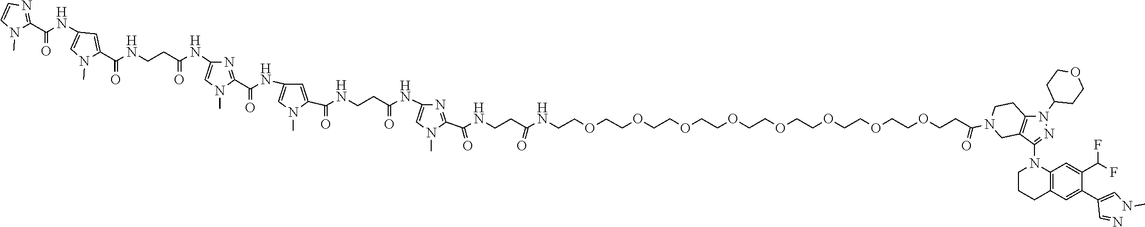 [SMILES: Cn1cc(-c2cc3c(cc2C(F)F)N(c2nn(C4CCOCC4)c4c2CN(C(=O)CCOCCOCCOCCOCCOCCOCCOCCOCCNC(=O)CCNC(=O)c2nc(NC(=O)CCNC(=O)c5cc(NC(=O)c6nc(NC(=O)CCNC(=O)c7cc(NC(=O)c8nccn8C)cn7C)cn6C)cn5C)cn2C)CC4)CCC3)cn1] |
| A−22 | 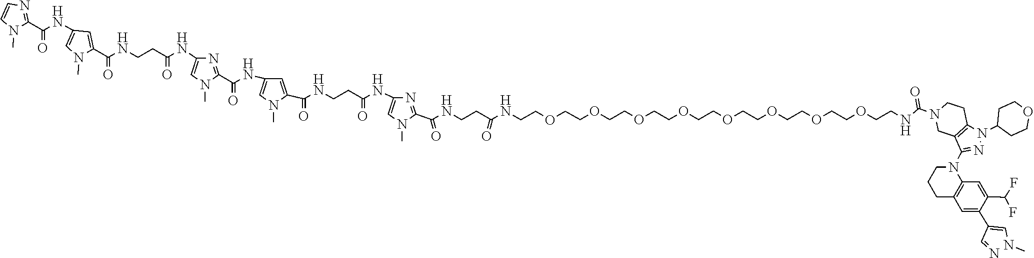 [SMILES: Cn1cc(-c2cc3c(cc2C(F)F)N(c2nn(C4CCOCC4)c4c2CN(C(=O)NCCOCCOCCOCCOCCOCCOCCOCCOCCNC(=O)CCNC(=O)c2nc(NC(=O)CCNC(=O)c5cc(NC(=O)c6nc(NC(=O)CCNC(=O)c7cc(NC(=O)c8nccn8C)cn7C)cn6C)cn5C)cn2C)CC4)CCC3)cn1] |
| A−23 | 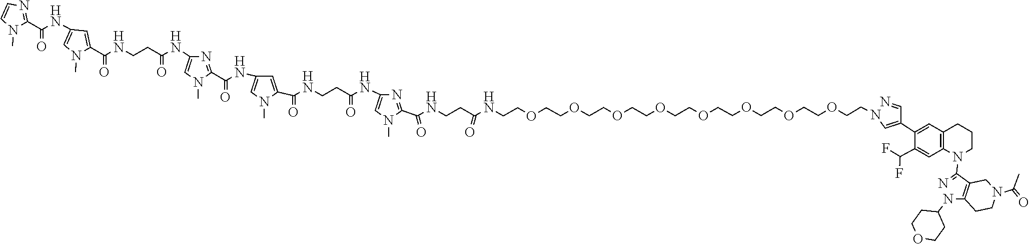 [SMILES: CC(=O)N1CCc2c(c(N3CCCc4cc(-c5cnn(CCOCCOCCOCCOCCOCCOCCOCCOCCNC(=O)CCNC(=O)c6nc(NC(=O)CCNC(=O)c7cc(NC(=O)c8nc(NC(=O)CCNC(=O)c9cc(NC(=O)c%10nccn%10C)cn9C)cn8C)cn7C)cn6C)c5)c(C(F)F)cc43)nn2C2CCOCC2)C1] |
| A−24 | 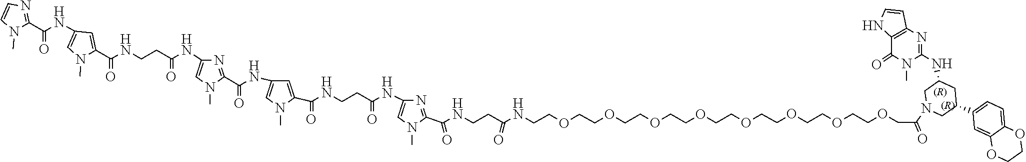 |
| A−25 | 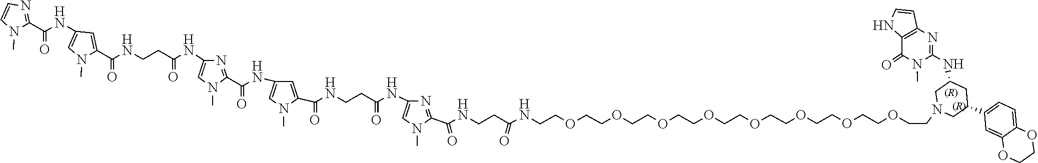 |
| A−26 | 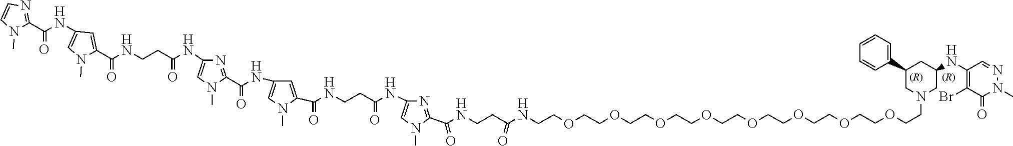 |
| A−27 | 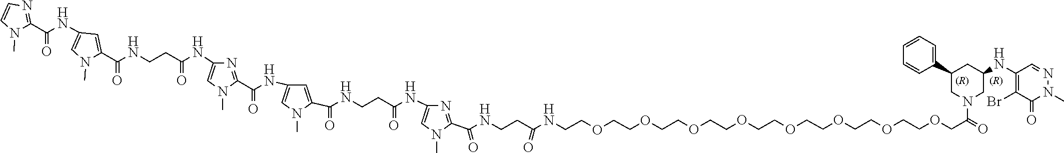 |
| A−28 | 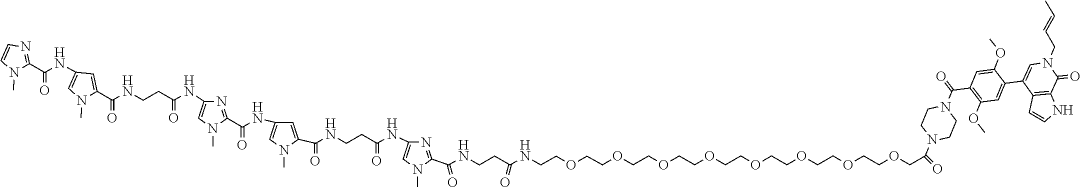 [SMILES: C/C=C/Cn1cc(-c2cc(OC)c(C(=O)N3CCN(C(=O)COCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)cc2OC)c2cc[nH]c2c1=O] |
| A−29 | 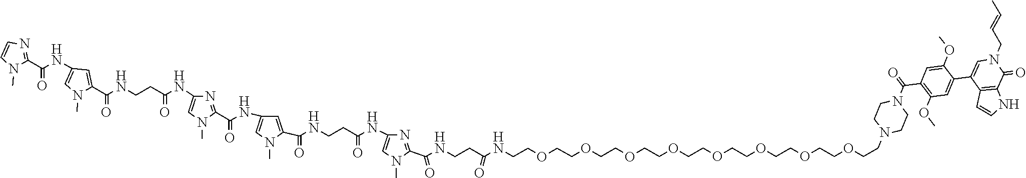 [SMILES: C/C=C/Cn1cc(-c2cc(OC)c(C(=O)N3CCN(CCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)cc2OC)c2cc[nH]c2c1=O] |
| A−30 | 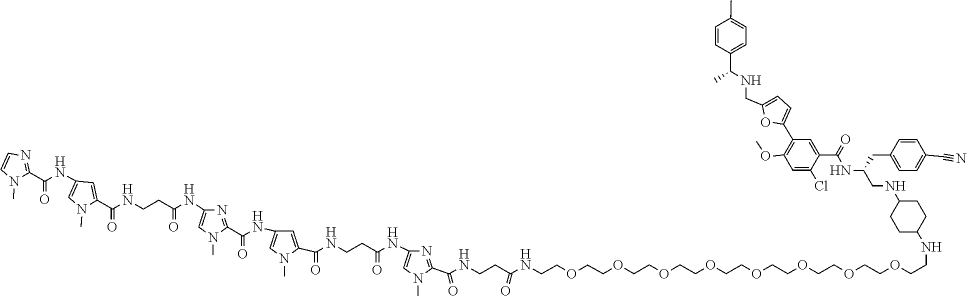 [SMILES: COc1cc(Cl)c(C(=O)N[C@@H](CNC2CCC(NCCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC2)Cc2ccc(C#N)cc2)cc1-c1ccc(CN[C@H](C)c2ccc(C)cc2)o1] |
| A−31 | 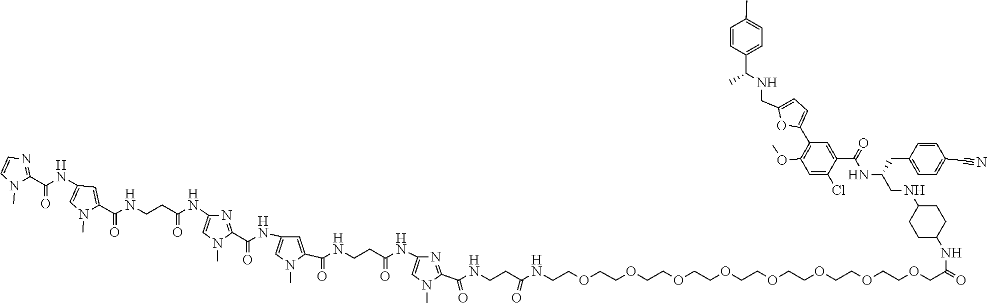 [SMILES: COc1cc(Cl)c(C(=O)N[C@@H](CNC2CCC(NC(=O)COCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC2)Cc2ccc(C#N)cc2)cc1-c1ccc(CN[C@H](C)c2ccc(C)cc2)o1] |
| A−32 | 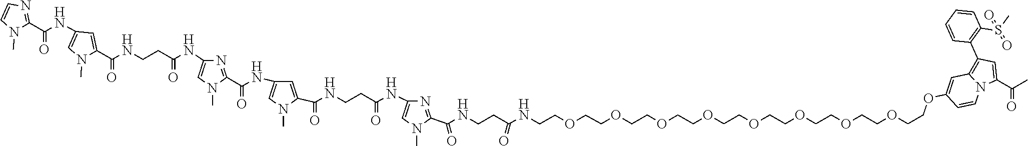 [SMILES: CC(=O)c1cc(-c2ccccc2S(C)(=O)=O)c2cc(OCCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)ccn12] |
| A−33 | 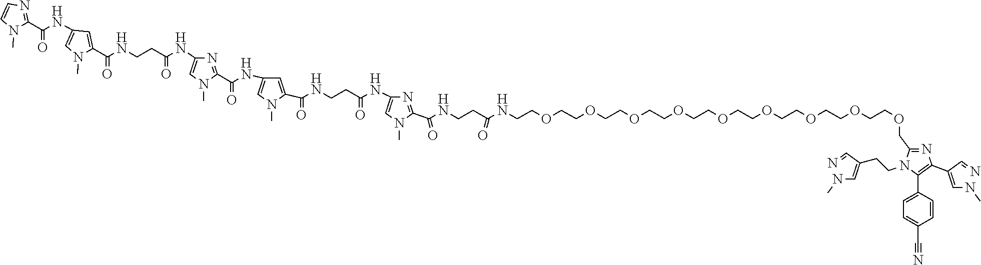 [SMILES: Cn1cc(CCn2c(COCCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)nc(-c3cnn(C)c3)c2-c2ccc(C#N)cc2)cn1] |
| A−34 | 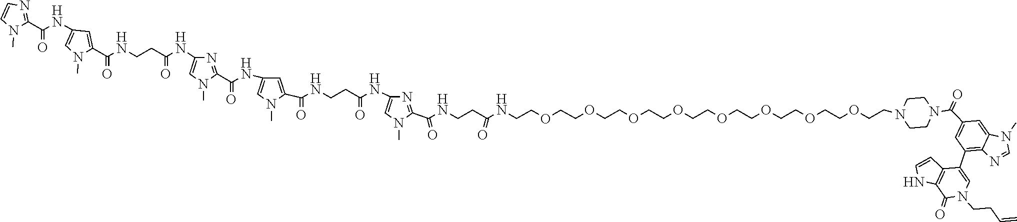 [SMILES: C=CCCn1cc(-c2cc(C(=O)N3CCN(CCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)cc3c2ncn3C)c2cc[nH]c2c1=O] |
| A−35 | 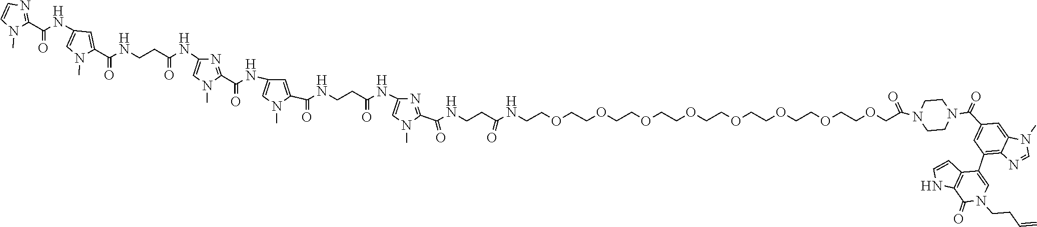 [SMILES: C=CCCn1cc(-c2cc(C(=O)N3CCN(C(=O)COCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)cc3c2ncn3C)c2cc[nH]c2c1=O] |

### Further Forms of the Compounds

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography.

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H (D), ³H, ¹³C, ¹⁴C, N, ¹⁸O, ⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% by molar. In some embodiments, one or more of the substituents disclosed herein comprise deuterium at a percentage higher than the natural abundance of deuterium. In some embodiments, one or more ¹H are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N*(C₁-C₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

In some embodiments, the compounds described herein exist as solvates. In some embodiments, the disclosure provides for methods of treating diseases by administering the compounds in the form of such solvates. In some embodiments, the disclosure provides for methods of treating diseases by administering a composition comprising the compounds in the form of such solvates. Solvates contain either stoichiometric or non-stoichiometric amounts of a solvent, and, in some embodiments, are formed during the process of crystallization with pharmaceutically acceptable solvents.

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Methods of Use

In another aspect, provided herein is a method of treating a subject having an expanded GAA repeat disorder, the method comprising administering a transcriptional modulator molecule having a first terminus, a second terminus, and an oligomeric backbone, wherein
(a) the first terminus comprises a DNA-binding moiety capable of binding a nucleotide repeat of GAA;
(b) the second terminus comprises a protein-binding moiety capable of binding to a regulatory molecule that modulates expression of a gene having the expanded GAA repeat; and
(c) the oligomeric backbone links the first terminus and the second terminus.

In some embodiments, the expanded GAA repeat has at least about 36 repeats, at least about 40 repeats, at least about 50 repeats, at least about 60 repeats, at least about 70 repeats, at least about 80 repeats, at least about 90 repeats, at least about 100 repeats, at least about 110 repeats, at least about 120 repeats, or more.

In another aspect, provided herein is a method of modulating the transcription of fxn in a subject in need thereof, comprising the step of contacting fxn with a transcriptional modulator molecule having a first terminus, a second terminus, and an oligomeric backbone, wherein
(a) the first terminus comprises a DNA-binding moiety capable of binding a nucleotide repeat of GAA;
(b) the second terminus comprises a protein-binding moiety capable of binding to a regulatory molecule that modulates expression of a gene having the expanded GAA repeat; and
(c) the oligomeric backbone links the first terminus and the second terminus.

The cell phenotype, cell proliferation, transcription of fxn, production of mRNA from transcription of fxn, translation of fxn, change in biochemical output produced by the protein coded by fxn, or noncovalent binding of the protein coded by fxn with a natural binding partner may be monitored. Such methods may be modes of treatment of disease, biological assays, cellular assays, biochemical assays, or the like. In some embodiments, ex vivo methods of treatment are provided. Ex vivo methods typically include cells, organs, and/or tissues removed from the subject. The cells, organs and/or tissues can, for example, be incubated with the agent under appropriate conditions. The contacted cells, organs, and/or tissues are typically returned to the donor, placed in a recipient, or stored for future use.

The transcription modulator molecules described herein can recruit the regulatory molecule to modulate the expression of the defective fxn gene and effectively treat and/or and alleviate the symptoms associated with diseases such as Friedreich's ataxia.

In some embodiments, administration of the molecules described herein modulates expression of fxn within 6 hours of treatment. In some embodiments, administration of the molecules modulates expression of fxn within 24 hours of treatment. In some embodiments, administration of the molecules modulates expression of fxn within 72 hours of treatment.

In some embodiments, administration of the molecule described herein causes a 2-fold increase in expression of fxn. In some embodiments, administration of the molecule causes a 5-fold increase in expression of fxn. In some embodiments, administration of the molecule causes a 10-fold increase in expression of fxn. In some embodiments, administration of the molecule causes a 20-fold increase in expression of fxn.

In some embodiments, administration of a molecule described herein causes a 20% decrease in expression of fxn. In some embodiments, administration of the molecule causes a 50% decrease in expression of fxn. In some embodiments, administration of the molecule causes a 80% decrease in expression of fxn. In some embodiments, administration of the molecule causes a 90% decrease in expression of fxn. In some embodiments, administration of the molecule causes a 95% decrease in expression of fxn. In some embodiments, administration of the molecule causes a 99% decrease in expression of fxn.

In some embodiments, administration of a molecule described herein causes expression of fxn to fall within 25% of the level of expression observed for a healthy subject. In some embodiments, administration of the molecule causes expression of fxn to fall within 50% of the level of expression observed for a healthy subject. In some embodiments, administration of the molecule causes expression of fxn to fall within 75% of the level of expression observed for a healthy subject In some embodiments, administration of the molecule causes expression of fxn to fall within 90% of the level of expression observed for a healthy subject.

In another aspect, provided herein is a method of treating Friedreich's ataxia in a subject in need thereof, comprising administering to the subject a therapeutically effective dose of a transcription modulator molecule having a first terminus, a second terminus, and an oligomeric backbone.

In some embodiments, the method comprises alleviating one or more of muscular atrophy, ataxia, fasciculation, or dementia.

### Pharmaceutical Compositions and Administration

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, (N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The dose of a pharmaceutical agent described herein for treating a disease or disorder may depend upon the subject's condition, that is, stage of the disease, severity of symptoms caused by the disease, general health status, as well as age, gender, and weight, and other factors apparent to a person skilled in the medical art. Pharmaceutical compositions may be administered in a manner appropriate to the disease to be treated as determined by persons skilled in the medical arts. In addition to the factors described herein and above related to use of pharmaceutical agent for treating a disease or disorder, suitable duration and frequency of administration of the pharmaceutical agent may also be determined or adjusted by such factors as the condition of the patient, the type and severity of the patient's disease, the particular form of the active ingredient, and the method of administration. Optimal doses of an agent may generally be determined using experimental models and/or clinical trials. The optimal dose may depend upon the body mass, weight, or blood volume of the subject. The use of the minimum dose that is sufficient to provide effective therapy is usually preferred. Design and execution of pre-clinical and clinical studies for a pharmaceutical agent, including when administered for prophylactic benefit, described herein are well within the skill of a person skilled in the relevant art. When two or more pharmaceutical agents are administered to treat a disease or disorder, the optimal dose of each pharmaceutical agent may be different, such as less than when either agent is administered alone as a single agent therapy. In certain particular embodiments, two pharmaceutical agents in combination may act synergistically or additively, and either agent may be used in a lesser amount than if administered alone. An amount of a pharmaceutical agent that may be administered per day may be, for example, between about 0.01 mg/kg and 100 mg/kg, e.g., between about 0.1 to 1 mg/kg, between about 1 to 10 mg/kg, between about 10-50 mg/kg, between about 50-100 mg/kg body weight. In other embodiments, the amount of a pharmaceutical agent that may be administered per day is between about 0.01 mg/kg and 1000 mg/kg, between about 100-500 mg/kg, or between about 500-1000 mg/kg body weight. The optimal dose, per day or per course of treatment, may be different for the disease or disorder to be treated and may also vary with the administrative route and therapeutic regimen.

### Definitions

Unless defined otherwise, all technical and scientific terms used herein have the same meaning as is commonly understood by one of skill in the art to which this invention belongs.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The terms below, as used herein, have the following meanings, unless indicated otherwise:
“oxo” refers to ═O.
“Carboxyl” refers to —COOH.
“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —C(═O)OH, —C(═O)OMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —C(═O)OH, C(═O)OMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —C(═O)OH, C(═O)OMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —C(═O)OH, C(═O)OMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system can contain only hydrogen and carbon and from five to eighteen carbon atoms, where at least one of the rings in the ring system is aromatic, i.e., it contains a cyclic, delocalized (4n+2) π-electron system in accordance with the Hickel theory. The ring system from which aryl groups are derived include, but are not limited to, groups such as benzene, fluorene, indane, indene, tetralin and naphthalene. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —C(═O)OH, C(═O)OMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Carbocycle” refers to a saturated, unsaturated, or aromatic rings in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated, and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle may be optionally substituted.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —C(═O)OH, C(═O)OMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Cycloalkenyl” refers to an unsaturated non-aromatic monocyclic or poly cyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls includes, e.g., cyclopentenyl, cyclohexenyl, cycloheptenyl, and cyclooctenyl.

“Halo” or “halogen” refers to bromo, chloro, fluoro or iodo. In some embodiments, halogen is fluoro or chloro. In some embodiments, halogen is fluoro.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di-and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro, 2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₆ heterocycloalkenyl), from two to five carbon atoms (e.g., C₂-C₅ fully saturated heterocycloalkyl or C₂-C₅ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered fully saturated heterocycloalkyl. In some embodiments, the heterocycloalkyl is a 3- to 7-membered fully saturated heterocycloalkyl. In some embodiments, the heterocycloalkyl is a 3- to 6-membered fully saturated heterocycloalkyl. In some embodiments, the heterocycloalkyl is a 4- to 6-membered fully saturated heterocycloalkyl. In some embodiments, the heterocycloalkyl is a 5- to 6-membered fully saturated heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —C(═O)OH, C(═O)OMe. —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —C(═O)OH, C(═O)OMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “polyamide” refers to polymers of linkable units chemically bound by amide (i.e., CONH) linkages; optionally, polyamides include chemical probes conjugated therewith. Polyamides may be synthesized by stepwise condensation of carboxylic acids (COOH) with amines (RR′NH) using methods known in the art. Alternatively, polyamides may be formed using enzymatic reactions in vitro, or by employing fermentation with microorganisms.

The term “linkable unit” refers to methylimidazoles, methylpyrroles, and straight and branched chain aliphatic functionalities (e.g., methylene, ethylene, propylene, butylene, and the like) which optionally contain nitrogen Substituents, and chemical derivatives thereof. The aliphatic functionalities of linkable units can be provided, for example, by condensation of B-alanine or dimethylaminopropylamine during synthesis of the polyamide by methods well known in the art.

The term “linker” or “oligomeric backbone” refers to a chain of at least 10 contiguous atoms. In certain embodiments, the linker contains no more than 20 non-hydrogen atoms. The terms linker and oligomeric backbone can be used interchangeably. In some embodiments, the linker contains no more than 40 non-hydrogen atoms. In some embodiments, the linker contains no more than 60 non-hydrogen atoms. In certain embodiments, the linker contains atoms chosen from C, H, N, O, and S. In some embodiments, every non-hydrogen atom is chemically bonded either to 2 neighboring atoms in the linker, or one neighboring atom in the linker and a terminus of the linker. In some embodiments, the linker forms an amide bond with at least one of the two other groups to which it is attached. In certain embodiments, the linker forms an ester or ether bond with at least one of the two other groups to which it is attached. In some embodiments, the linker forms a thioester or thioether bond with at least one of the two other groups to which it is attached. In some embodiments, the linker forms a direct carbon-carbon bond with at least one of the two other groups to which it is attached. In some embodiments, the linker forms an amine or amide bond with at least one of the two other groups to which it is attached. In some embodiments, the linker comprises —(CH₂OCH₂)— units. In some embodiments, the linker comprises —(CH(CH₃)OCH₂)— units. In some embodiments, the linker comprises —(CH₂NR_{N}CH₂) units, for R_{N}═C₁-C₄alkyl. In some embodiments, the linker comprises an arylene, cycloalkylene, or heterocycloalkylene moiety.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

As used herein, “optionally substituted” is a substituted group is derived from the unsubstituted parent group in which there has been an exchange of one or more hydrogen atoms for another atom or group. Unless otherwise indicated, when a group is deemed to be “substituted” or “optionally substituted” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

The term “oligonucleotide sequence” refers to a plurality of nucleic acids having a defined sequence and length (e.g., 2, 3, 4, 5, 6, or even more nucleotides). The term “oligonucleotide repeat sequence” refers to a contiguous expansion of oligonucleotide sequences.

The term “transcription,” well known in the art, refers to the synthesis of RNA (i.e., ribonucleic acid) by DNA-directed RNA polymerase. The term “modulate transcription” refers to a change in transcriptional level which can be measured by methods well known in the art, for example, assay of mRNA, the product of transcription. In certain embodiments, modulation is an increase in transcription. In other embodiments, modulation is a decrease in transcription.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

An “effective amount” or “therapeutically effective amount” refers to an amount of a compound administered to a mammalian subject, either as a single dose or as part of a series of doses, which is effective to produce a desired therapeutic effect.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “contacting” refers to bringing the compound (e.g. a transcription molecular molecule of the present disclosure) into proximity of the desired target gene. The contacting may result in the binding to or result in a conformational change of the target moiety.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Compound Synthesis

The following examples are intended to illustrate but not limit the disclosed embodiments. The transcription modulator molecule such as those listed in Table 4 can be prepared using the synthesis.

Compounds of the present disclosure can be prepared using methods illustrated in general synthetic schemes and experimental procedures detailed below. General synthetic schemes and experimental procedures are presented for purposes of illustration and are not intended to be limiting. Starting materials used to prepare compounds of the present disclosure are commercially available or can be prepared using routine methods known in the art.

Synthetic chemistry transformations and methodologies useful in synthesizing the compounds described herein are known in the art and include, for example, those described in R.

Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Abbreviations

Ac₂O=acetic anhydride; AcCl=acetyl chloride; AcOH=acetic acid; AIBN=azobisisobutyronitrile; aq.=aqueous; Bu₃SnH=tributyltin hydride; CD₃OD=deuterated methanol; CDCl₃=deuterated chloroform; CDI=1,1′-Carbonyldiimidazole; DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; DPPA=diphenylphosphoryl azide; EDC.HCl=EDCI.HCl=1-ethyl-3-(3-dimethylaminopropyl)carbodiimide hydrochloride; Et₂O=diethyl ether; EtOAc=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; i-PrOH=isopropanol; LAH=lithium aluminium hydride; LiHMDS=Lithium bis(trimethylsilyl)amide; MeCN=acetonitrile; MeOH=methanol; MP-carbonate resin=macroporous triethylammonium methylpolystyrene carbonate resin; MsCl=mesyl chloride; MTBE=methyl tertiary butyl ether; MW=microwave irradiation; n-BuLi=n-butyllithium; NaHMDS=Sodium bis(trimethylsilyl)amide; NaOMe=sodium methoxide; NaOtBu=sodium t-butoxide; NBS=N-bromosuccinimide; NCS=N-chlorosuccinimide; NMP=N-Methyl-2-pyrrolidone; Pd(Ph₃)₄=tetrakis(triphenylphosphine)palladium(0); Pd₂(dba)₃=tris(dibenzylideneacetone)dipalladium(0); PdCl₂(PPh₃)₂=bis(triphenylphosphine)palladium(11) dichloride; PG=protecting group; prep-HPLC=preparative high-performance liquid chromatography; PyBop=(benzotriazol-1-yloxy)-tripyrrolidinophosphonium hexafluorophosphate; Pyr=pyridine; RT=room temperature; RuPhos=2-dicyclohexylphosphino-2′,6′-diisopropoxybiphenyl; sat.=saturated; ss=saturated solution; t-BuOH=tert-butanol; T3P=Propylphosphonic Anhydride; TBS=TBDMS=tert-butyldimethylsilyl; TBSCI=TBDMSCl=tert-butyldimethylchlorosilane; TEA=Et₃N=triethylamine; TFA=trifluoroacetic acid; TFAA=trifluoroacetic anhydride; THF=tetrahydrofuran; Tol=toluene; TsCl=tosyl chloride; XPhos=2-dicyclohexylphosphino-2′,4′,6′-triisopropylbiphenyl.

### Synthesis of Representative Polyamides (DNA-Binding Moiety)

### Example 1. Synthesis of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH)

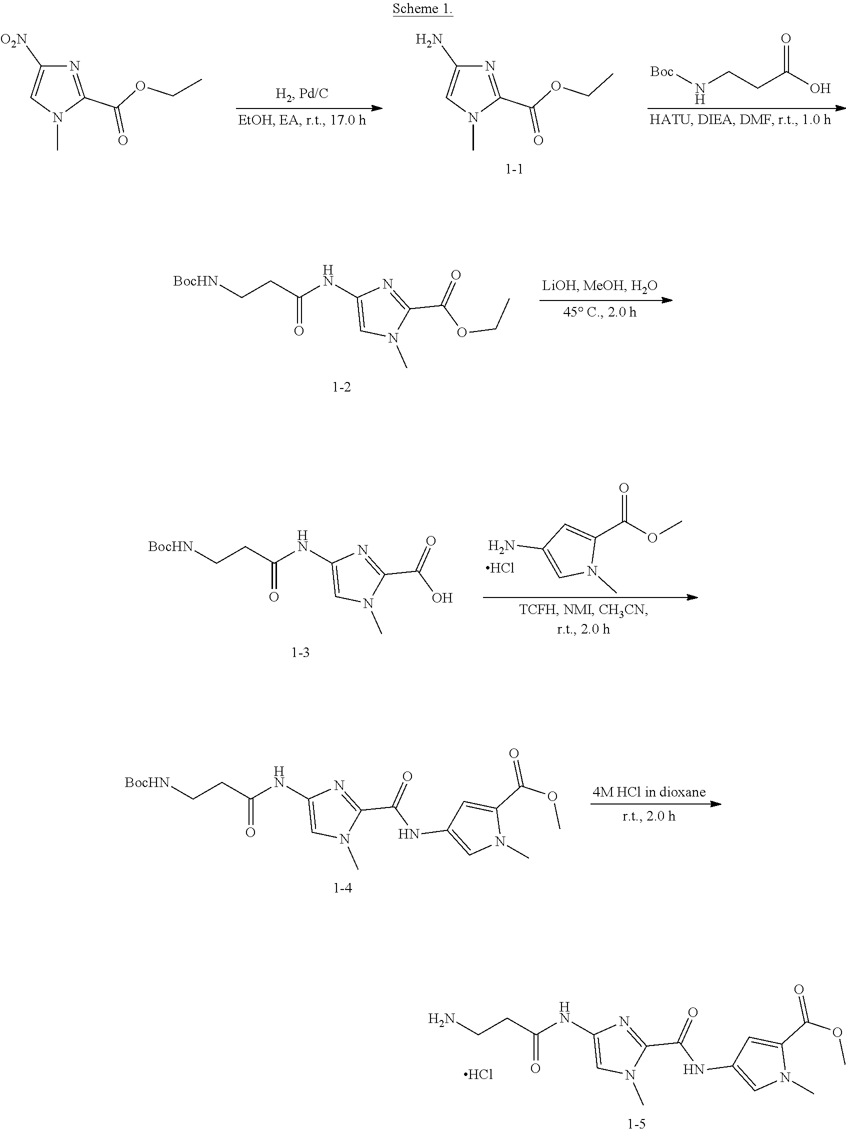

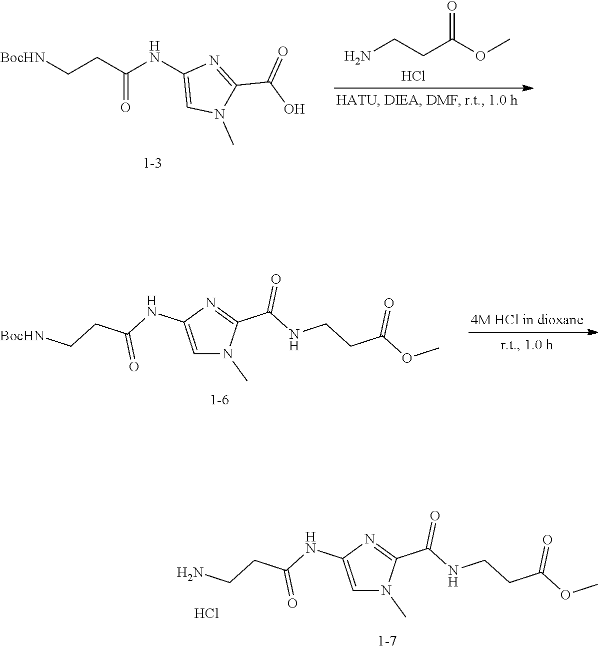

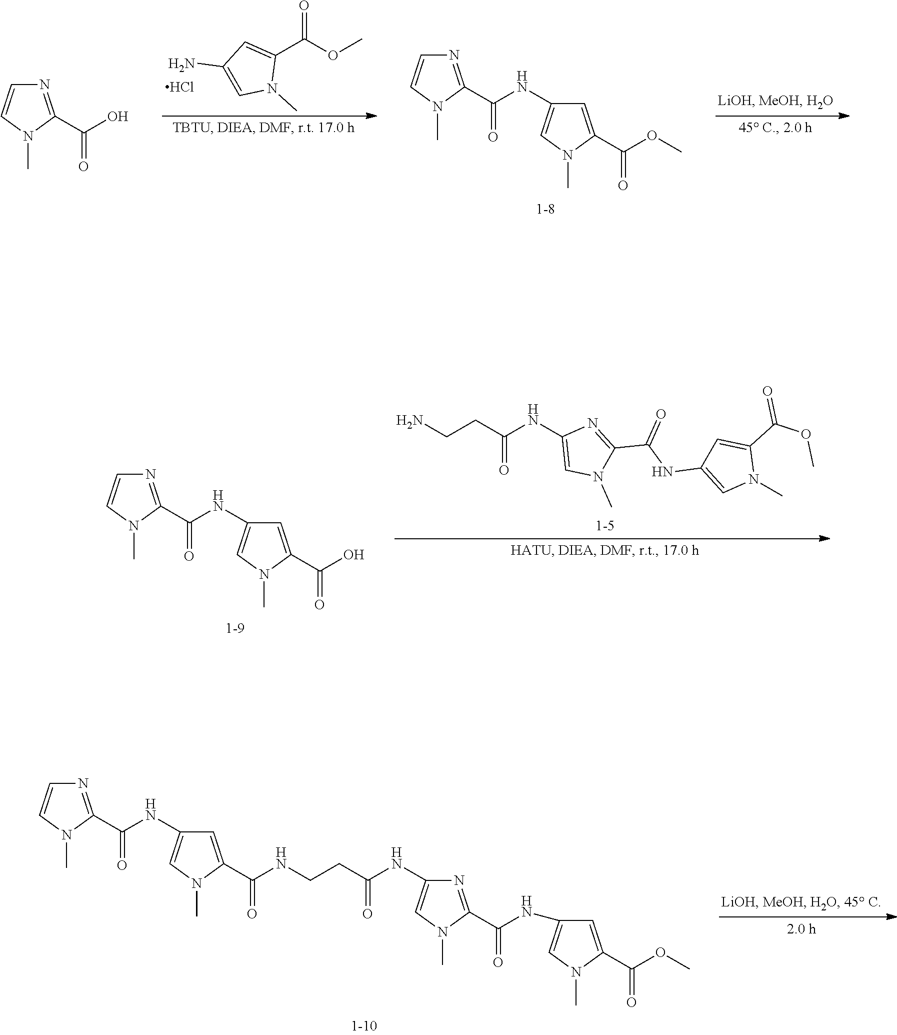

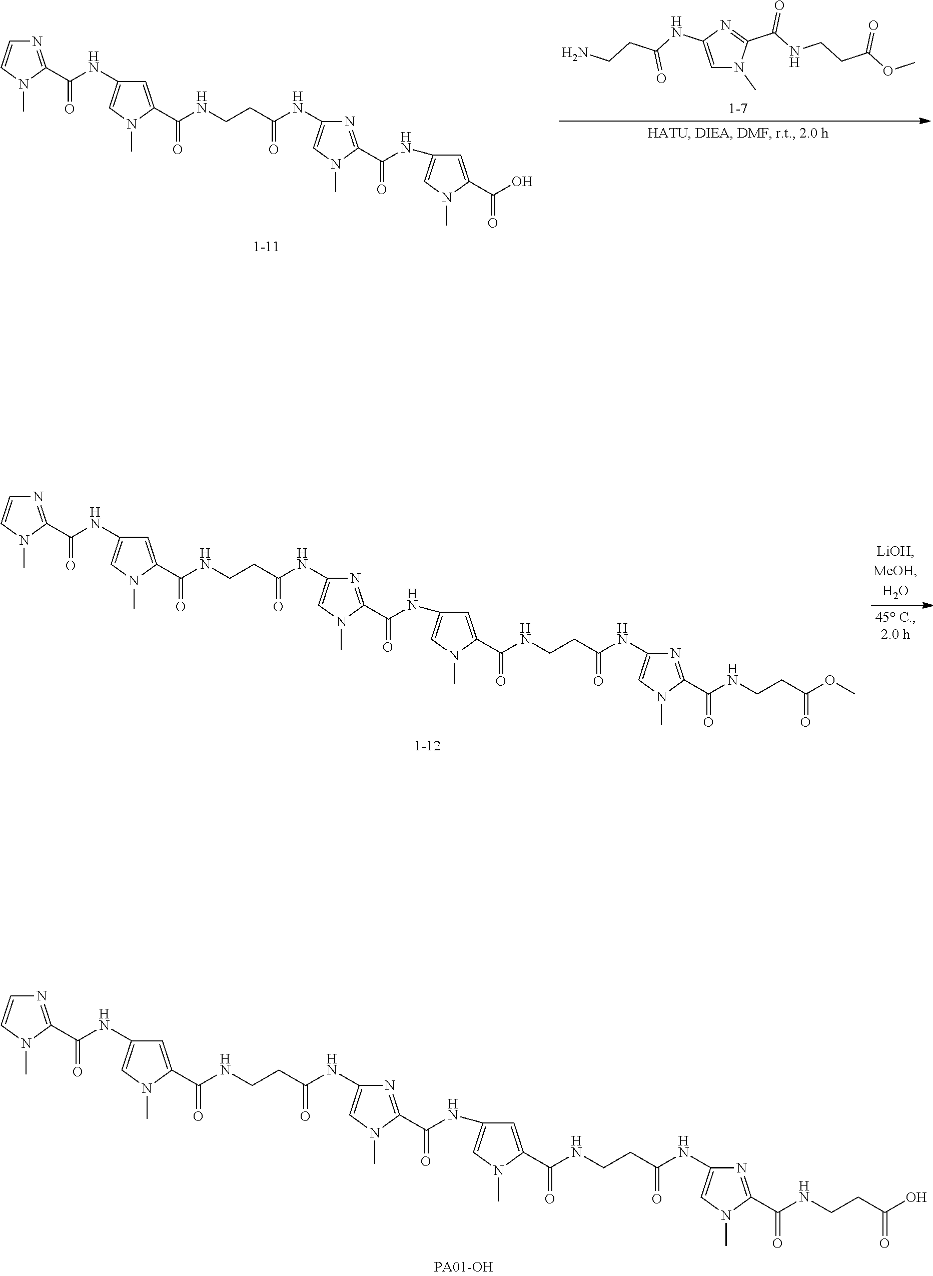 [SMILES: Cn1cc(NC(=O)c2nc(NC(=O)CCNC(=O)c3cc(NC(=O)c4nccn4C)cn3C)cn2C)cc1C(=O)O ; COC(=O)CCNC(=O)c1nc(NC(=O)CCN)cn1C ; COC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)O)n1]

### Step 1: Synthesis of ethyl 4-amino-1-methylimidazole-2-carboxylate

To a solution of ethyl 1-methyl-4-nitroimidazole-2-carboxylate (30.00 g, 150.63 mmol, 1.00 equiv) in E0H (120.00 mL) and EA (120.00 mL) was added Pd/C (8.01 g, 27% w/w). Then the reaction was stirred for 17.0 h at room temperature under H₂ atmosphere. The solid was filtrated out and the filtrate was concentrated to afford ethyl 4-amino-1-methylimidazole-2-carboxylate (22.30 g, 75.20%) as yellow solid. LC/MS: mass calcd. For C₇H₁₁N₃O₂: 169.09, found: 170.10 [M+H]⁺. ¹H NMR (400 MHz, DMSO-d₆) δ: 7.37 (s, 1H), 4.29-4.34 (m, 2H), 3.94 (s, 3H), 1.31 (t, J=7.2 Hz, 3H).

### Step 2: Synthesis of ethyl 4-[3-[(tert-butoxycarbonyl)amino]propanamido]-1-methylimidazole-2-carboxylate

Into a 500 mL flask was added 3-[(tert-butoxycarbonyl) amino]propanoic acid (22.45 g, 118.65 mmol, 0.90 equiv), DMF (180.00 mL). The mixture was cooled to 0 degrees C., then HATU (75.18 g, 197.71 mmol, 1.50 equiv) and DIEA (51.11 g, 395.43 mmol, 3.00 equiv) were added, the mixture was stirred for 10.0 mins, then ethyl 4-amino-1-methylimidazole-2-carboxylate (22.30 g, 131.81 mmol, 1.00 equiv) was added in portions. The reaction was stirred at room temperature for 1.0 h. The reaction was quenched with ice water (600 mL), and the solution was stirred for 15.0 min. The precipitated solids were collected by filtration and washed with water (3×50 mL) and dried under vacuum. This resulted in ethyl 4-[3-[(tert-butoxycarbonyl)amino]propanamido]-1-methylimidazole-2-carboxylate (34.50 g, 76.90%) as a light yellow solid. LC/MS: mass calcd. For C₁₅H₂₄N₄O₅: 340.17, found: 341.20 [M+H]⁺. H NMR (400 MHz, DMSO-d₆) δ: 10.63 (s, 1H), 7.52 (s, 1H), 6.80 (t, J=5.6 Hz, 1H), 4.23-4.28 (m, 2H), 3.90 (s, 3H), 3.15-3.20 (m, 2H), 2.42 (t, J=7.2 Hz, 2H), 1.37 (s, 9H), 1.29 (t, J=7.2 Hz, 3H).

### Step 3: Synthesis of 4-[3-[(Tert-butoxycarbonyl)amino]propanamido]-1-methylimidazole-2-carboxylic acid

To a stirred solution of ethyl 4-[3-[(tert-butoxycarbonyl)amino]propanamido]-1 Methylimidazole-2-carboxylate (34.50 g, 101.36 mmol, 1.00 equiv) in MeOH (200.00 mL) was added LiOH solution (2M, 202.00 mL, 4.00 equiv) dropwise at room temperature. The resulting mixture was stirred for 2.0 h at 45 degrees C. The resulting mixture was concentrated under reduced pressure. The residue was dissolved in H₂O (50 mL). The mixture was acidified to pH 3˜5 with 2M HCl. The precipitated solids were collected by filtration and washed with H₂O (3×30 mL), dried under vacuum. 4-[3-[(Tert-butoxycarbonyl)amino]propanamido]-1-methylimidazole-2-carboxylic acid (30.00 g, 94.77%) was obtained as white solid. LC/MS: mass calcd. For C₁₃H₂₀N₄O₅: 312.14, found: 313.15 [M+H]⁺. ¹H NMR (300 MHz, DMSO-d₆) δ: 10.53 (s, 1H), 7.48 (s, 1H), 6.79 (t, J=5.4 Hz, 1H), 3.89 (s, 3H), 3.15-3.22 (m, 2H), 2.43 (t, J=7.2 Hz, 2H), 1.37 (s, 9H).

### Step 4: Synthesis of Methyl 4-(4-[3-[(tert-butoxycarbonyl)amino]propanamido]-1-methylimidazole-2-amido)-1-methylpyrrole-2-carboxylate

To a stirred solution of 4-[3-[(tert-butoxycarbonyl)amino]propanamido]-1-methylimidazole-2-carboxylic acid (16.00 g, 51.23 mmol, 1.00 equiv) in CH₃CN (150.00 mL) was added TCFH (21.56 g, 76.84 mmol, 1.50 equiv), NM1 (12.62 g, 153.69 mmol, 3.00 equiv) and methyl 4-amino-1-methylpyrrole-2-carboxylate hydrochloride (10.74 g, 56.34 mmol, 1.10 equiv) in portions at 0° C. The resulting mixture was stirred for 2.0 h at room temperature. The precipitated solids were collected by filtration and washed by CH₃CN (3×20 mL), dried under vacuum. Methyl 4-(4-[3-[(tert-butoxycarbonyl)amino]propanamido]-1-methylimidazole-2-amido)-1-methylpyrrole-2-carboxylate (19.00 g, 82.70%) was obtained as white solid. LC/MS: mass calcd. For C₂₀H₂₈N₆O₆: 448.21, found: 449.25 [M+H]⁺. ¹H NMR (300 MHz, DMSO-d₆) δ: 10.24 (s, 1H), 10.11 (s, 1H), 7.52 (s, 1H), 7.33 (s, 1H), 6.99 (s, 1H), 6.82 (t, J=5.1 Hz, 1H), 3.94 (s, 3H), 3.85 (s, 3H), 3.74 (s, 3H), 3.16-3.23 (m, 2H), 2.47 (t, J=6.9 Hz, 2H), 1.38 (s, 9H).

### Step 5: Synthesis of Methyl 4-[4-(3-aminopropanamido)-1-methylimidazole-2-amido]-1-methylpyrrole-2-carboxylate hydrochloride

A solution of methyl 4-(4-[3-[(tert-butoxycarbonyl)amino]propanamido]-1-methylimidazole-2-amido)-1-methylpyrrole-2-carboxylate (19.00 g, 42.37 mmol, 1.00 equiv) in HCl/1,4-dioxane (4M, 200.00 mL) was stirred for 2.0 h at room temperature. The resulting mixture was concentrated under vacuum. Methyl 4-[4-(3-aminopropanamido)-1-methylimidazole-2-amido]-1-methylpyrrole-2-carboxylate hydrochloride (19.00 g crude) was obtained as a yellow solid. LC/MS: mass calcd. For C₁₅H₂₁ClN₆O₄: 348.15, found: 349.05 [M+H]⁺. H NMR (300 MHz, CD₃OD) δ: 7.37 (s, 2H), 6.91 (s, 1H), 4.03 (s, 3H), 3.88 (s, 3H), 3.79 (s, 3H), 3.09 (t, J=6.6 Hz, 2H), 2.64 (t, J=6.6 Hz, 2H).

### Step 6: Synthesis of methyl 3-[(4-[3-[(tert-butoxycarbonyl)amino]propanamido]-1-methylimidazol-2-yl)formamido]propanoate

Into a 1000 ml flask was added 4-[3-[(tert-butoxycarbonyl)amino]propanamido]-1-methylimidazole-2-carboxylic acid (11.00 g, 35.22 mmol. 1.00 equiv). DMF (300.00 mL), the mixture was cooled to 0° C., then HATU (20.09 g, 52.83 mmol, 1.50 equiv), DIEA (18.21 g, 140.88 mmol, 4.00 equiv) was added dropwise, the mixture was stirred for 10 mins, methyl 3-aminopropanoate (3.63 g, 35.22 mmol. 1.00 equiv) was added in portions. The reaction was stirred at room temperature for 1.0 h. The reaction mixture was poured into water/ice (600 mL), the solid was filtered out and dried under vacuum. The aqueous phase was extracted by EtOAc (3×200 mL), the organic phases were combined and washed by H₂O (1×200 mL) and NaCl (1×200 mL), dried over anhydrous Na₂SO₄. After filtration, the filtrate was concentrated under reduced pressure. The residue was purified by silica gel column, eluted with pure EtOAc. The fractions were combined and concentrated. Methyl 3-[(4-[3-[(tert-butoxycarbonyl)amino]propanamido]-1-methylimidazol-2-yl)formamido]propanoate (13.00 g, 87.95%) was obtained as a yellow solid. LC/MS: mass calcd. For C₁₇H₂₇N₅O₆: 397.20, found: 398.20 [M+H]⁺. H NMR (400 MHz, DMSO-d₆) δ: 10.28 (s, 1H), 7.92 (t, J=6.0 Hz, 1H), 7.37 (s, 1H), 6.77 (t, J=6.0 Hz, 1H), 3.88 (s, 3H), 3.59 (s, 3H), 3.42-3.47 (m, 2H), 3.13-3.18 (m, 2H), 2.56 (t, J=6.0 Hz, 2H), 2.42 (t, J=6.0 Hz, 2H), 1.35 (s, 9H).

### Step 7: Synthesis of methyl 3-[[4-(3-aminopropanamido)-1-methylimidazol-2-yl]formamido]propanoate hydrochloride

A solution of methyl 3-[(4-[3-[(tert-butoxycarbonyl)amino]propanamido]-1-methylimidazol-2-yl) formanido]propanoate (11.00 g, 27.678 mmol, 1.00 equiv) in HCl/1,4 dioxane (4M, 110.00 mL) was stirred for 1.0 h at room temperature. The resulting mixture was concentrated under vacuum to afford methyl 3-[[4-(3-aminopropanamido)-1-methylimidazol-2-yl]formamido]propanoate hydrochloride (11.00 g, crude) as a yellow oil. LC/MS: mass calcd. For C₁₂H₁₉N₅O₄: 297.14, found: 298.20 [M+H]⁺. ¹H NMR (400 MHz, DMSO-d₆) δ: 10.57 (s, 1H), 7.92 (t, J=6.0 Hz, 1H), 7.37 (s, 1H), 3.89 (s, 3H), 3.59 (s, 3H), 3.43-3.47 (m, 2H), 2.97-3.05 (m, 2H), 2.57-2.71 (m, 2H), 2.56 (t, J=6.0 Hz, 2H).

### Step 8: Synthesis of Methyl 1-methyl-4-(1-methylimidazole-2-amido)pyrrole-2-carboxylate

To a stirred solution of 1-methylimidazole-2-carboxylic acid (10.00 g, 79.29 mmol, 7.00 equiv) in DMF (150.00 mL) was added TBTU (38.19 g, 118.94 mmol, 1.50 equiv), methyl 4-amino-1-methylpyrrole-2-carboxylate hydrochloride (16.63 g, 87.24 mmol, 1.10 equiv) and DIEA (30.74 g, 237.88 mmol, 3.00 equiv) in portions at 0° C. The resulting mixture was stirred for 17.0 h at room temperature. The reaction was poured into water/Ice (450 mL). The precipitated solids were collected by filtration and washed with H₂O (3×50 mL), dried under vacuum. Methyl 1-methyl-4-(1-methylimidazole-2-amido)pyrrole-2-carboxylate (16.50 g, 78.37%) was obtained as a white solid. LC/MS: mass calcd. For C₁₂H₁₄N₄O₃: 262.11, found: 263.15 [M+H]⁺. ¹H NMR (300 MHz, DMSO-d₆) δ: 10.54 (s, 1H), 7.54 (s, 1H), 7.40 (s, 1H), 7.04 (s, 2H), 3.99 (s, 3H), 3.85 (s, 3H), 3.74 (s, 3H).

### Step 9: Synthesis of 1-methyl-4-(1-methylimidazole-2-amido)pyrrole-2-carboxylic acid

To a stirred solution of methyl 1-methyl-4-(1-methylimidazole-2-amido)pyrrole-2-carboxylate (16.50 g, 62.91 mmol, 1.00 equiv) in MeOH (100.00 mL) was added LiOH solution (2M, 158.00 mL, 5.00 equiv) dropwise at room temperature. The resulting mixture was stirred for 2.0 h at 45° C. The resulting mixture was concentrated under reduced pressure.

The residue was dissolved in H₂O (50 mL). The mixture was acidified to pH 3˜5 with 2M HCl. The precipitated solids were collected by filtration and washed with H₂O (3×30 mL), dried under vacuum. 1-Methyl-4-(1-methylimidazole-2-amido)pyrrole-2-carboxylic acid (12.00 g, 76.84%) was obtained as a white solid. LC/MS: mass calcd. For C₁₁H₁₂N₄O₃: 248.09, found: 249.10 [M+H]⁺. ¹H NMR (300 MHz, DMSO-d₆) δ: 10.52 (s, 1H), 7.48 (s, 1H), 7.41 (s, 1H), 7.06 (s, 1H), 6.99 (s, 1H), 3.99 (s, 3H), 3.82 (s, 3H).

### Step 10: Synthesis of methyl 1-methyl-4-[1-methyl-4-(3-[[1-methyl-4-(1-methylimidazole-2-amido)pyrrol-2-yl]formamido]propanamido)imidazole-2-amido]pyrrole-2-carboxylate

To a stirred solution of 1-methyl-4-(1-methylimidazole-2-amido)pyrrole-2-carboxylic acid (9.00 g, 36.255 mmol, 1.00 equiv) in DMF (150.00 mL) was added HATU (20.68 g, 54.38 mmol, 1.50 equiv), DIEA (14.06 g, 108.77 mmol, 3.00 equiv) and methyl 4-[4-(3-aminopropanamido)-1-methylimidazole-2-amido]-1-methylpyrrole-2-carboxylate (13.89 g, 39.872 mmol, 1.10 equiv) in portions at 0° C. The resulting mixture was stirred for 17.0 h at room temperature. The reaction was poured into water/Ice (450 mL) at 0° C. The precipitated solids were collected by filtration and washed with H₂O (3×50 mL), dried under vacuum. Methyl 1-methyl-4-[1-methyl-4-(3-[[1-methyl-4-(1-methylimidazole-2-amido)pyrrol-2-yl]formamido]propanamido)imidazole-2-amido]pyrrole-2-carboxylate (14.00 g, 63.54%) was obtained as a yellow solid. LC/MS: mass calcd. For C₂₆H₃₀N₁₀O₆: 578.23, found: 579.10 [M+H]⁺. ¹H NMR (300 MHz, DMSO-d₆) δ: 10.53 (s, 1H), 10.29 (s, 1H), 10.11 (s, 1H), 8.10 (t, J=5.4 Hz, 1H), 7.52 (s, 1H), 7.47 (s, 2H), 7.25 (s, 1H), 7.17 (s, 1H), 6.99 (s, 1H), 6.97 (s, 1H), 3.99 (s, 3H), 3.95 (s, 3H), 3.84 (s, 3H), 3.82 (s, 3H), 3.69 (s, 3H), 3.42-3.49 (m, 2H), 2.60 (t, J=7.2 Hz, 2H).

### Step 11: Synthesis of 1-methyl-4-[1-methyl-4-(3-[[1-methyl-4-(I-methylimidazole-2-amido)pyrrol-2-yl]formamido]propanamido)imidazole-2-affordamido]pyrrole-2-carboxylic acid

A solution of methyl 1-methyl-4-[1-methyl-4-(3-[[1-methyl-4-(1-methylimidazole-2-amido)pyrrol-2-yl|formamido|propanamido)imidazole-2-amido|pyrrole-2-yl]formamidocarboxylate (14.00 g, 24.20 mmol, 1.00 equiv) in MeOH (70.00 mL) was added LiOH (2M, 72.00 mL, 6.00 equiv). The mixture was stirred at 45° C. for 2.0 h. The resulting mixture was concentrated under reduced pressure. The residue was dissolved in H₂O (50 mL). The mixture was acidified to pH 3˜5 with 2 M HCl. The precipitated solids were collected by filtration and washed with H₂O (3×20 mL), dried under vacuum. 1-methyl-4-[1-methyl-4-(3-[[1-methyl-4-(1-methylimidazole-2-amido)pyrrol-2-yl]formamido]propanamido)imidazole-2-affordamido]pyrrole-2-carboxylic acid (12.00 g, 81.49%) was obtained as a yellow solid. LC/MS: mass calcd. For C₂₅H₂₈N₁₀O₆: 564.22, found: 565.15[M+H]⁺. ¹H NMR (300 MHz, DMSO-d₆) δ: 10.72 (s, 1H), 10.32 (s, 1H), 10.08 (s, 1H), 8.14 (t, J=6.0 Hz, 1H), 7.51 (s, 1H), 7.47 (s, 2H), 7.27 (s, 1H), 7.23 (s, 1H), 6.98 (s, 1H), 6.94 (s, 1H), 4.00 (s, 3H), 3.95 (s, 3H), 3.82 (s, 6H), 3.44-3.46 (m, 2H), 2.60 (t, J=6.6 Hz, 2H).

### Step 12: Synthesis of methyl 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoate

To a stirred solution of 1-methyl-4-[1-methyl-4-(3-[[1-methyl-4-(1-methylimidazole-2-amido)pyrrol-2-yl]formamido]propanamido)imidazole-2-amido]pyrrole-2-carboxylic acid (12.00 g, 21.26 mmol, 1.00 equiv) in DMF (100.00 mL) was added HATU (12.12 g, 31.88 mmol, 1.50 equiv), DIEA (8.24 g, 63.77 mmol, 3.00 equiv) and methyl 3-[[4-(3-aminopropanamido)-1-methylimidazol-2-yl]formamido]propanoate (6.95 g, 23.38 mmol, 1.10 equiv) in portions at 0° C. The resulting mixture was stirred for 2.0 h at room temperature. The reaction was poured into water/ice (300 mL) at 0° C. The precipitated solids were collected by filtration and washed with H2O (3×30 mL), dried under vacuum. Methyl 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoate (13.00 g, 64.77%) was obtained as a yellow solid. LC/MS: mass calcd. For C₃₇H₄₅N₁₅O₉: 843.35, found: 844.55[M+H]⁺. ¹H NMR (300 MHz, DMSO-d₆) δ: 10.41 (s, 1H), 10.37 (s, 1H), 10.32 (s, 1H), 9.96 (s, 1H), 8.08 (s, 2H), 7.96 (s, 1H), 7.46 (s, 1H), 7.42 (s, 1H), 7.38 (s, 1H), 7.24 (s, 2H), 7.03 (s, 1H), 6.98 (s, 1H), 6.93 (s, 1H), 4.13 (s, 3H), 3.98 (s, 3H), 3.95 (s, 3H), 3.81 (s, 9H), 3.60 (s, 6H), 2.57-2.69 (m, 6H).

### Step 13: Synthesis of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid

A solution of methyl 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoate (10.00 g, 10.59 mmol, 1.00 equiv) in MeOH (60.00 mL) was added 2M LiOH (21.20 mL, 42.40 mmol, 4.00 equiv), the resulting mixture was stirred for 2.0 h at 45° C. The resulting mixture was concentrated under reduced pressure. The resulting mixture was diluted with water (60 mL). The mixture was acidified to pH 3˜5 with 2M HCl. The precipitated solids were collected by filtration and washed with water (3×20 mL). The solid was dried under vacuum. This resulted in 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (8.70 g, 84.14%) as a brown solid. LC/MS: mass calcd. For C₃₆H₄₃N₁₅O₉: 829.34, found: 830.25[M+H]⁺. ¹H NMR (300 MHz, DMSO-d₆) δ: 10.46 (s, 1H), 10.39 (s, 1H), 10.31 (s, 1H), 9.93 (s, 1H), 8.05-8.10 (m, 2H), 7.87 (t, J=6.0 Hz, 1H), 7.42-7.46 (m, 3H), 7.20-7.23 (m, 2H), 7.07 (s, 1H), 6.90-6.95 (m, 2H), 3.95 (s, 3H), 3.92 (s, 3H), 3.89 (s, 3H), 3.79 (s, 3H), 3.78 (s, 3H), 3.38-3.41 (m, 6H), 2.44-2.59 (m, 6H).

### Example 2. Synthesis of 1-methyl-4-[3-({l-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxylic acid

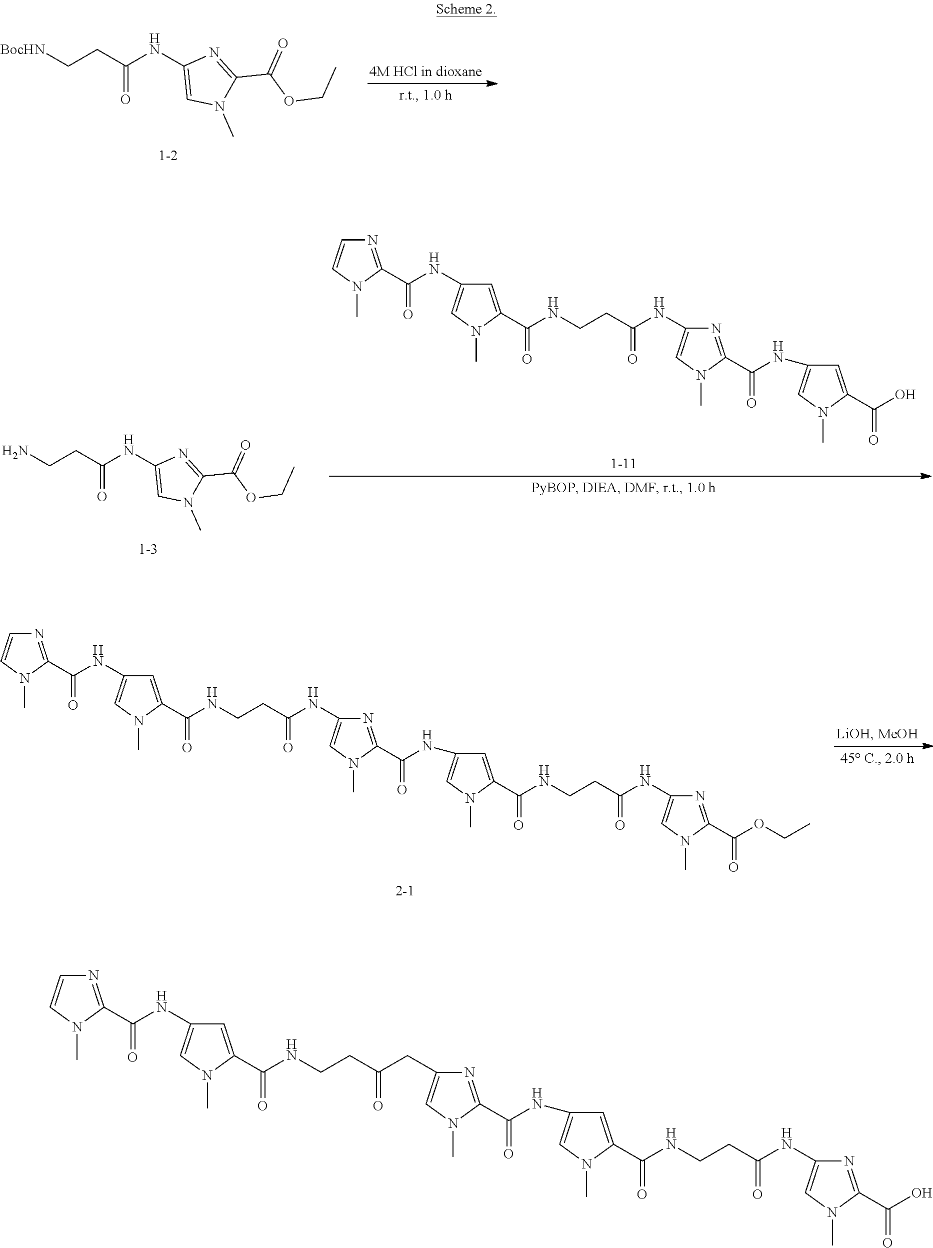 [SMILES: CCOC(=O)c1nc(NC(=O)CCNC(=O)OC(C)(C)C)cn1C ; Cn1cc(NC(=O)c2nc(NC(=O)CCNC(=O)c3cc(NC(=O)c4nccn4C)cn3C)cn2C)cc1C(=O)O ; CCOC(=O)c1nc(NC(=O)CCN)cn1C ; CCOC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C ; Cn1cc(NC(=O)c2nccn2C)cc1C(=O)NCCC(=O)Cc1cn(C)c(C(=O)Nc2cc(C(=O)NCCC(=O)Nc3cn(C)c(C(=O)O)n3)n(C)c2)n1]

### Step 1: Synthesis of 2-(1-methylimidazol-2-yl)-3H-1,3-benzodiazole-5-carboxylic acid

The procedure was the same as (Example 1 Step 7), but the reaction time was 1.0 h. 2.00 g of ethyl 4-[3-[(tert-butoxycarbonyl)amino]propanamido]-1-methylimidazole-2-carboxylate was used, 2.00 g crude of ethyl 4-(3-aminopropanamido)-1-methyl-1H-imidazole-2-carboxylate was obtained as an off-white solid. LC/MS: mass calcd. For C₁₀H₁₆N₄O₃: 240.12, found: 241.10 [M+H]⁺.

### Step 2: Synthesis of ethyl 1-methyl-4-[3-({I-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxylate

The procedure was the same as methyl 4-(4-{4-[(tert-butoxycarbonyl)amino]-1-methylpyrrole-2-amido}-1-methylimidazole-2-amido)-1-methylpyrrole-2-carboxylate. 270.00 mg of 1-methyl-4-[1-methyl-4-(3-{[1-methyl-4-(1-methylimidazole-2-amido)pyrrol-2-yl]formamido}propanamido)imidazole-2-amido]pyrrole-2-carboxylic acid was used, 460.00 mg of ethyl 1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxylate was obtained as an off-white solid (96.45% yield). LC/MS: mass calcd. For C₃₅H₄₂N₄O₅: 786.33, found: 809.60 [M+Na].

### Step 3: Synthesis of 1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxylic acid

The procedure was the same as 4-[3-[(tert-butoxycarbonyl)amino]propanamido]-1-methylimidazole-2-carboxylic acid (Example 1 Step 3). 470.00 mg of ethyl 1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxylate was used, 400.00 mg of 1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxylic acid was obtained as an off-white solid (74.41% yield). LC/MS: mass calcd. For C₃₃H₃₈N₄O₈:758.30, found: 759.55 [M+H]⁺.

### SYNTHESIS OF REPRESENTATIVE LIGANDS

### Example 3. Synthesis of 2-chloro-4-{2-[3-(cyclohexylamino)-7-(3,5-dimethyl-1,2-oxazol-4-yl)imidazo[1,2-a]pyridin-2-yl]ethyl}phenol

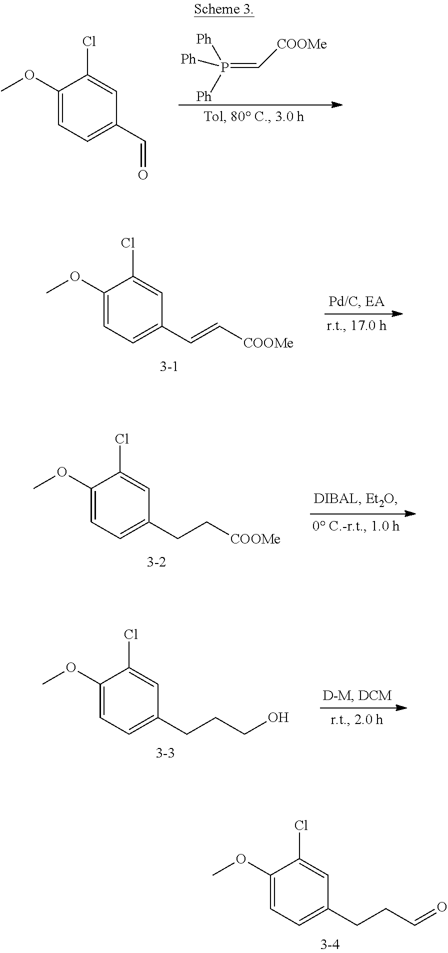 [SMILES: COc1ccc(C=O)cc1Cl ; COC(=O)C=P(c1ccccc1)(c1ccccc1)c1ccccc1 ; COC(=O)/C=C/c1ccc(OC)c(Cl)c1 ; COC(=O)CCc1ccc(OC)c(Cl)c1 ; COc1ccc(CCCO)cc1Cl ; COc1ccc(CCC=O)cc1Cl]

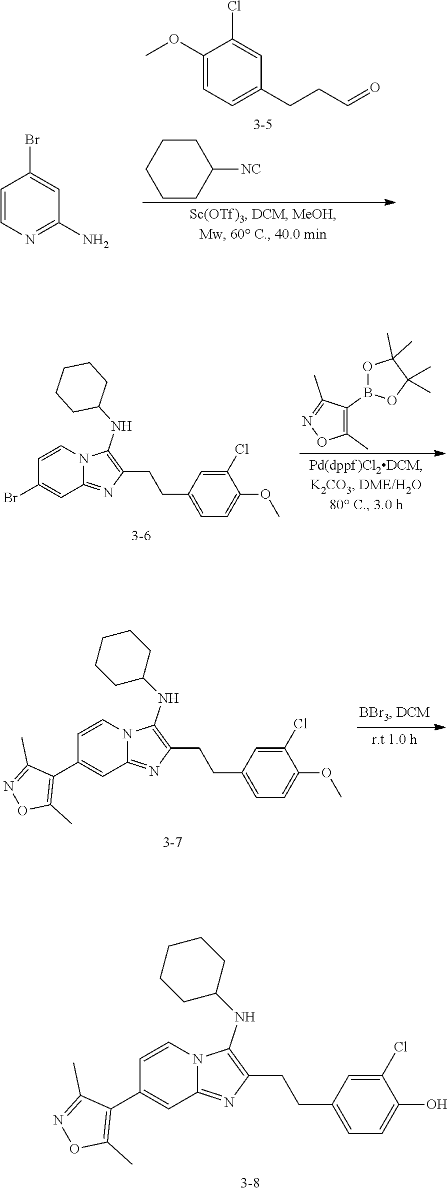

### Step 1: Synthesis of methyl (2E)-3-(3-chloro-4-methoxyphenyl)prop-2-enoate

A solution of 3-chloro-4-methoxybenzaldehyde (35.00 g, 205.17 mmol, 1.00 equiv) and methyl 2-(triphenyl-lambda5-phosphanylidene)acetate (52.82 g, 157.98 mmol, 0.77 equiv) in toluene (400.00 mL) was stirred for 3.0 h at 80° C. The resulting mixture was concentrated under vacuum. The residue was purified by silica gel column chromatography, eluted with PE/EA (1:2) to afford methyl (2E)-3-(3-chloro-4-methoxyphenyl)prop-2-enoate (43.00 g, 70.25%) as white solid. LC/MS: mass calcd. For C₁₁H₁₁ClO₃: 226.04 found: 226.95 [M+H]⁺.

### Step 2: Synthesis of methyl 3-(3-chloro-4-methoxyphenyl)propanoate

To a solution of methyl (2E)-3-(3-chloro-4-methoxyphenyl)prop-2-enoate (14 g) in EA (25.00 mL) was added Pd/C (25% w/w). Then the reaction was stirred for 2.0 h at room temperature under H₂ atmosphere. The mixture was filtrated and the filtrate was concentrated to afford methyl 3-(3-chloro-4-methoxyphenyl)propanoate, (14 g) as colorless oil (81.63% yield). LC/MS: mass calcd. For C₁₁H₁₃ClO₃: 228.06, found: 228.95 [M+H]⁺.

### Step 3: Synthesis of 3-(3-chloro-4-methoxyphenyl)propan-1-ol

A solution of methyl 3-(3-chloro-4-methoxyphenyl)propanoate (20.00 g, 87.46 mmol, 1.00 equiv) in Et₂O (200.00 mL) followed by the addition of DIBAL (2M in Tol) (66.00 mL, 132.00 mmol, 1.50 equiv) dropwise at 0° C. The resulting mixture was stirred for 1.0 h at room temperature. The reaction was quenched by the addition of sat. NH₄CL (aq.) (200 mL) at 0° C. The resulting mixture was filtered and the filter cake was washed with CH₂Cl₂ (200 mL×2). The aqueous layer was extracted with CH₂Cl₂ (200 mL×2). The combined organic layers were dried over anhydrous Na₂SO₄. After filtration, the filtrate was concentrated under reduced pressure. The residue was purified by silica gel column chromatography, eluted with PE/EA (1:1) to afford 3-(3-chloro-4-methoxyphenyl)propan-1-ol (14.00 g, 79.77%) as colorless oil. LC/MS: mass calcd. For C₁₀H₁₃ClO₂: 200.06, found: 242.05 [M+H+ACN]⁺.

### Step 4: Synthesis of 3-(3-chloro-4-methoxyphenyl)propanal

A solution of 3-(3-chloro-4-methoxyphenyl)propan-1-ol (10.00 g, 49.83 mmol, 1.00 equiv) in DCM (100.00 mL) was added D-M reagent (24.50 g, 57.76 mmol, 1.16 equiv) in portions at 0° C. under nitrogen atmosphere. The resulting mixture was stirred for 2.0 h at room temperature. The resulting mixture was concentrated under reduced pressure. The residue was purified by silica gel column chromatography, eluted with PE/EA (1:1) to afford 3-(3-chloro-4-methoxyphenyl)propanal (5.60 g, 56.57%) as a yellow oil. LC/MS: mass calcd. For C₁₀H₁₁ClO₂: 198.04, found: 199.00 [M+H]⁺.

### Step 5: Synthesis of 7-bromo-2-[2-(3-chloro-4-methoxyphenyl)ethyl]-N-cyclohexylimidazo[1,2-a]pyridin-3-amine

A mixture of 4-bromopyridin-2-amine (4.00 g, 23.12 mmol, 1.00 equiv) and 3-(3-chloro-4-methoxyphenyl)propanal (3.84 g, 19.33 mmol, 0.84 equiv), cyclohexyl isocyanide (2.40 g, 21.98 mmol, 0.95 equiv), scandium(III) bis(trifluoromethanesulfonate) (1.00 g, 2.03 mmol, 0.09 equiv) in DCM (20.00 mL) and MeOH (20.00 mL). The reaction mixture was irradiated with microwave radiation for 40.0 min at 60° C. After the reaction, the reaction mixture was concentrated. The residue was purified by silica gel column chromatography, eluted with PE/EA (10:1) to afford 7-bromo-2-[2-(3-chloro-4-methoxyphenyl)ethyl]-N-cyclohexylimidazo[1,2-a]pyridin-3-amine (9.40 g, 87.85%) as brown solid. The reaction was proceed on 1.0 g×4 scale. LC/MS: mass calcd. C₂₂H₂₅BrClN₃O: 461.09, found: 462.20, 464.20 [M+H, M+H+2]⁺.

### Step 6: Synthesis of 2-[2-(3-chloro-4-methoxyphenyl)ethyl]-N-cyclohexyl-7-(3,5-dimethyl-1,2-oxazol-4-yl)imidazo[1,2-a]pyridin-3-amine

A solution of 7-bromo-2-[2-(3-chloro-4-methoxyphenyl)ethyl]-N-cyclohexylimidazo[1,2-a]pyridin-3-amine (11.00 g, 23.76 mmol, 1.00 equiv) and 3,5-dimethyl-4-(4,4,5,5-tetramethyl-1,3,2-dioxaborolan-2-yl)-1,2-oxazole (6.89 g, 30.89 mmol, 1.30 equiv), Pd(dppf)Cl₂ (1.74 g, 2.38 mmol, 0.10 equiv), K₂CO₃ (6.57 g, 47.534 mmol, 2.00 equiv) in DME (55.00 mL) and H₂O (55.00 mL) was stirred for 3.0 h at 80° C. After filtration, the filtrate was concentrated under reduced pressure. The resulting mixture was extracted with EtOAc (100 mL×3). The combined organic layers were washed with water (50 mL×2), dried over anhydrous Na₂SO₄. The residue was purified by silica gel column chromatography, eluted with CH₂Cl₂/MeOH (10:1) to afford 2-[2-(3-chloro-4-methoxyphenyl)ethyl]-N-cyclohexyl-7-(3,5-dimethyl-1,2-oxazol-4-yl)imidazo[1,2-a]pyridin-3-amine (9.00 g, 79.05%) as yellow solid. LC/MS: mass calcd. For C₂₇H₃₁ClN₄O₂: 478.21, found: 479.20 [M+H]⁺.

### Step 7: Synthesis of 2-chloro-4-{2-[3-(cyclohexylamino)-7-(3,5-dimethyl-1,2-oxazol-4-yl)imidazo[1,2-a]pyridin-2-yl]ethyl}phenol

A solution of 2-[2-(3-chloro-4-methoxyphenyl)ethyl]-N-cyclohexyl-7-(3,5-dimethyl-1,2-oxazol-4-yl)imidazo[1,2-a]pyridin-3-amine (7.34 g, 15.32 mmol, 1.00 equiv) in DCM (75.00 mL) was added BBr₃ (1M in DCM, 31.00 mL, 31.00 mmol, 5.00 equiv) dropwise at 0° C. Then the reaction mixture was stirred for 1.0 h at room temperature. The reaction was quenched with MeOH (50 mL) at 0° C. The resulting mixture was concentrated under vacuum. The obtain solid was washed with MeOH (50 mL). The precipitated solids were collected by filtration and washed with water (50 mL×3) and dried under vacuum. The resulting solid was purified with ACN/H₂O (1:1) (150 mL×3) and dried under vacuum. This resulted in 2-chloro-4-{2-[3-(cyclohexylamino)-7-(3,5-dimethyl-1,2-oxazol-4-yl)imidazo[1,2-a]pyridin-2-yl]ethyl}phenol (6.02 g, 79.20%) as yellow solid. LC/MS: mass calcd. For C₂₆H₂₉ClN₄O₂: 464.20, found: 465.20 [M+H]⁺.

### Example 4. Synthesis of tert-butyl N-[4-({2-[2-(3-chloro-4-methoxyphenyl)ethyl]-7-(3,5-dimethyl-1,2-oxazol-4-yl)imidazo[1,2-a]pyridin-3-yl}amino)cyclohexyl]carbamate

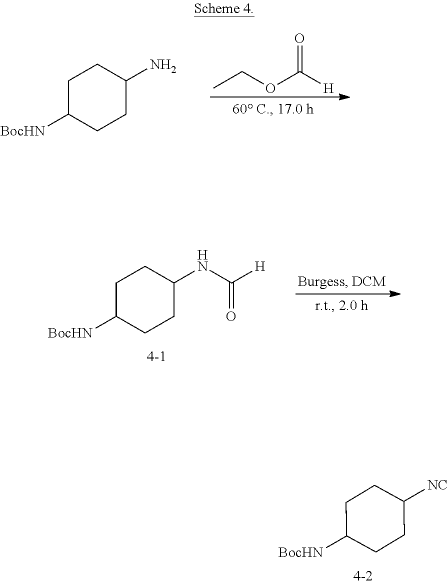

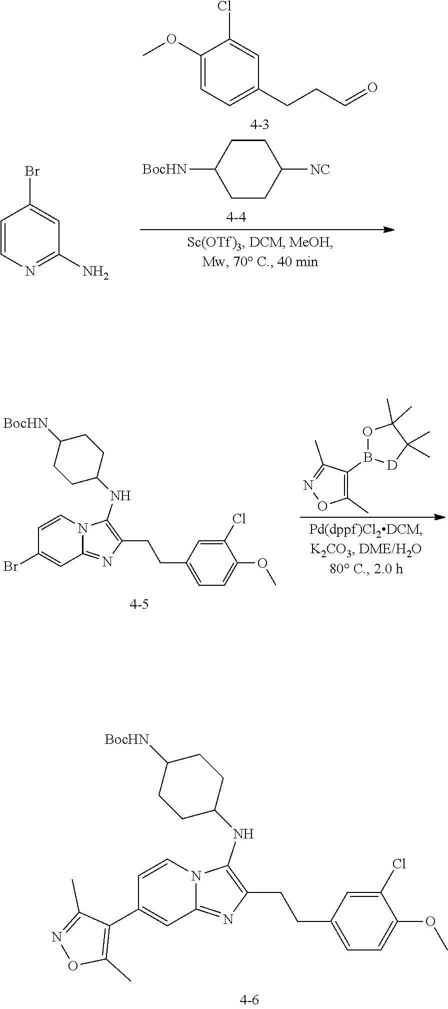

### Step 1: Synthesis of tert-butyl N-(4-formamidocyclohexyl)carbamate

A mixture of tert-butyl N-(4-aminocyclohexyl)carbamate (20.00 g, 93.32 mmol, 1.00 equiv) in ethyl formate (300.00 mL) was stirred for 17.0 h at 60° C. The mixture was allowed to cool to room temperature. The resulting mixture was concentrated under reduced pressure and the residue was purified with PE/EA (1:1) (150 mL). The solids were collected by filtration and washed with PE/EA=1:1 solution (20 mL×2). This resulted in tert-butyl N-(4-formamidocyclohexyl)carbamate (14.00 g, 61.91%) as an off-white solid. LC/MS: mass calcd. For C₁₂H₂₂N₂O₃: 242.16, found: 265.00 [M+Na]+.

### Step 2: Synthesis of N-(tert-butoxycarbonyl)-4-isocyanocyclohexan-1-amine

A solution of tert-butyl N-(4-formamidocyclohexyl)carbamate (14.00 g, 57.77 mmol, 1.00 equiv) and Burgess reagent (20.65 g, 86.66 mmol, 1.50 equiv) in DCM (280.00 mL) was stirred for 2.0 h at room temperature. The resulting mixture was concentrated under reduced pressure. The residue was purified by silica gel column chromatography, eluted with PE/EA (1:1) to afford N-(tert-butoxycarbonyl)-4-isocyanocyclohexan-1-amine (11.00 g, 84.88%) as white solid. LC/MS: mass calcd. For C₁₀H₁₁ClO₂: 224.15, found: 225.10 [M+H]⁺.

### Step 3: Synthesis of tert-butyl N-[4-({7-bromo-2-[2-(3-chloro-4-methoxyphenyl)ethyl]imidazo[1,2-a]pyridin-3-yl}amino)cyclohexyl]carbamate

The procedure was the same as 7-bromo-2-[2-(3-chloro-4-methoxyphenyl)ethyl]-N-cyclohexylimidazo[1,2-a]pyridin-3-amine (Example 3 step 5). 6.00 g of 3-(3-chloro-4-methoxyphenyl)propanal was used, 11.00 g of desired product was obtained as yellow solid (54.70% yield). LC/MS: mass calcd. For C₂₇H₃₄BrClN₄O₃: 576.15, found: 576.90, 578.90 [M+H, M+H+2]⁺. Note: the reaction was paralleled for 6 batches.

### Step 4: Synthesis of tert-butyl N-[4-({2-[2-(3-chloro-4-methoxyphenyl)ethyl]-7-(3,5-dimethyl-1,2-oxazol-4-yl)imidazo[1,2-a]pyridin-3-yl}amino)cyclohexyl]carbamate

The procedure was the same as 2-[2-(3-chloro-4-methoxyphenyl)ethyl]-N-cyclohexyl-7-(3,5-dimethyl-1,2-oxazol-4-yl)imidazo[1,2-a]pyridin-3-amine (Example 3 step 6), but the reaction time was 2.0 h and the residues was purified by revere phase column. 7.20 g of tert-butyl N-[4-({7-bromo-2-[2-(3-chloro-4-methoxyphenyl)ethyl]imidazo[1,2-a]pyridin-3-yl}amino)cyclohexyl]carbamate was used, 5.38 g, of desired product was obtained as green solid (69.44% yield). LC/MS: mass calcd. For C₃₂H₄₇ClN₉O₄: 593.28, found: 594.25 [M+H]⁺.

### Example 5. Synthesis of (S)-2-chloro-4-(2-(5-(3,5-dimethylisoxazol-4-yl)-1-(2-morpholinopropyl)-1H-benzo[d]imidazol-2-yl)ethyl)phenol

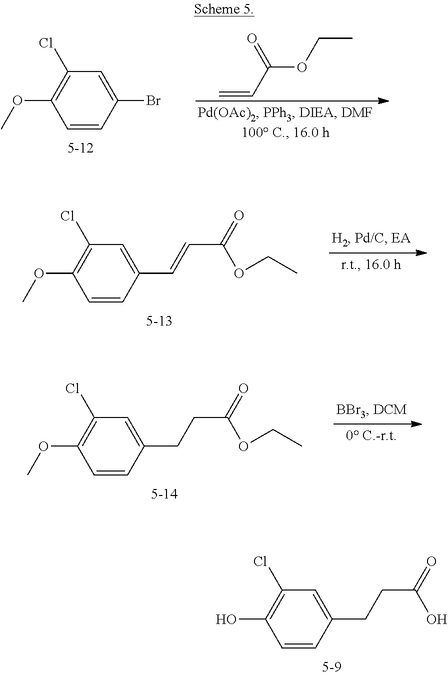

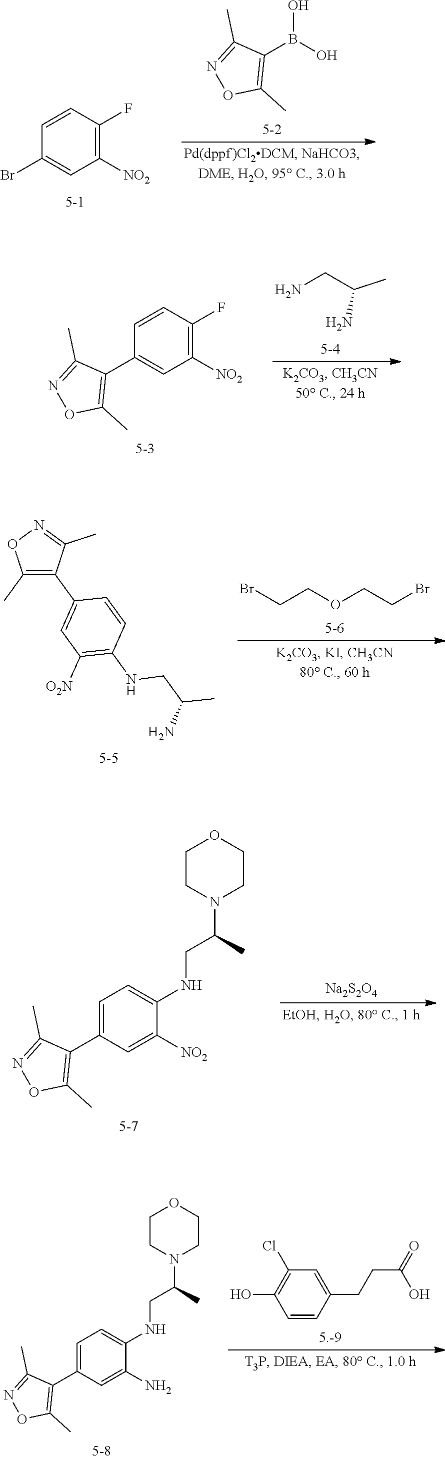

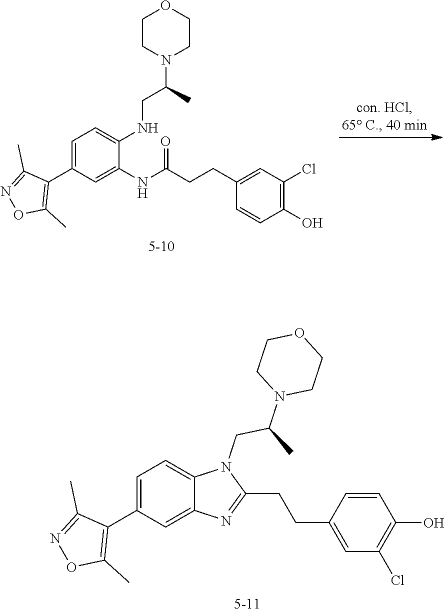 [SMILES: Cc1noc(C)c1-c1ccc(NC[C@H](C)N2CCOCC2)c(NC(=O)CCc2ccc(O)c(Cl)c2)c1 ; Cc1noc(C)c1-c1ccc2c(c1)nc(CCc1ccc(O)c(Cl)c1)n2C[C@H](C)N1CCOCC1]

### (S)-2-chloro-4-(2-(5-(3,5-dimethylisoxazol-4-yl)-1-(2-morpholinopropyl)-1H-benzo[d]imidazol-2-yl)ethyl)phenol was made by the methods and steps described in scheme 5

### Synthesis of Representative Compounds

### Example 6. Synthesis of Compound A-1

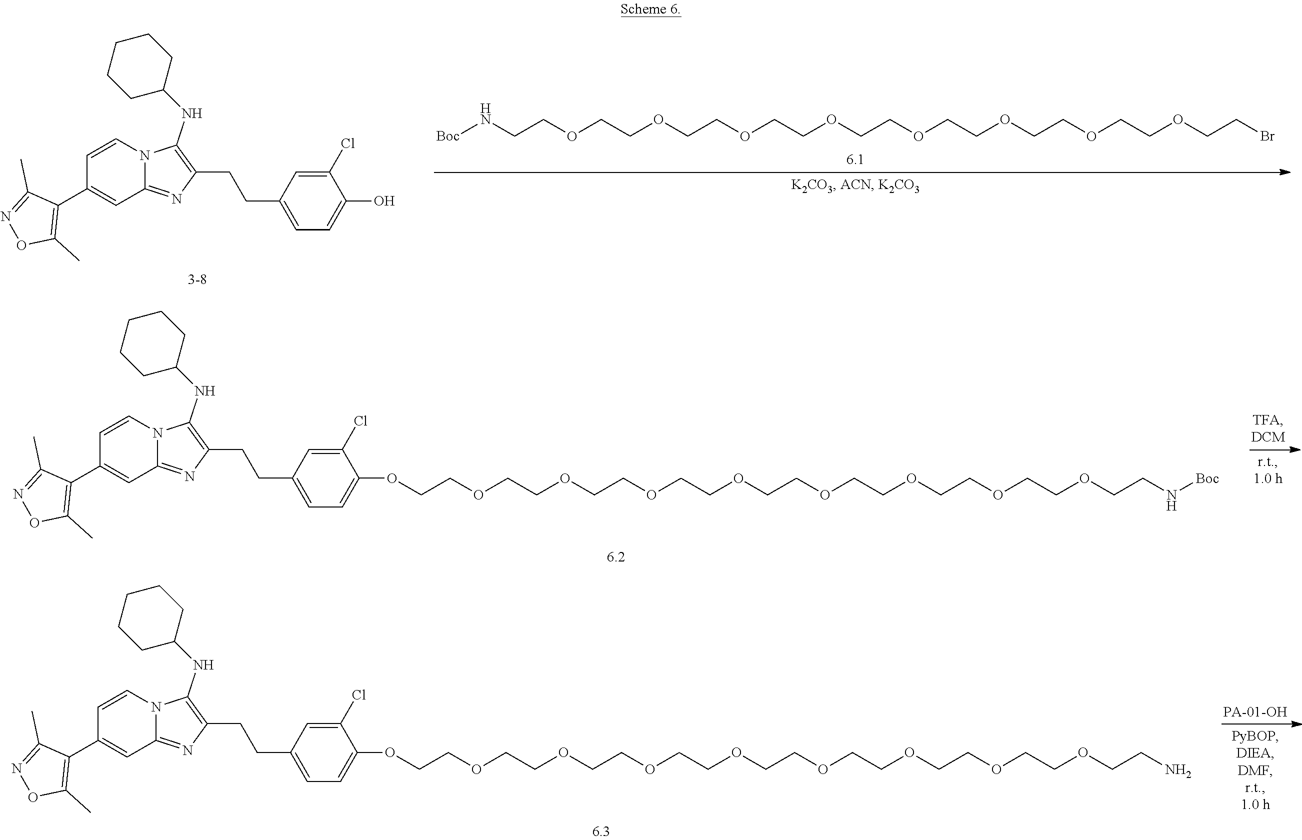 [SMILES: Cc1noc(C)c1-c1ccn2c(NC3CCCCC3)c(CCc3ccc(O)c(Cl)c3)nc2c1 ; CC(C)(C)OC(=O)NCCOCCOCCOCCOCCOCCOCCOCCOCCBr ; Cc1noc(C)c1-c1ccn2c(NC3CCCCC3)c(CCc3ccc(OCCOCCOCCOCCOCCOCCOCCOCCOCCNC(=O)OC(C)(C)C)c(Cl)c3)nc2c1 ; Cc1noc(C)c1-c1ccn2c(NC3CCCCC3)c(CCc3ccc(OCCOCCOCCOCCOCCOCCOCCOCCOCCN)c(Cl)c3)nc2c1]

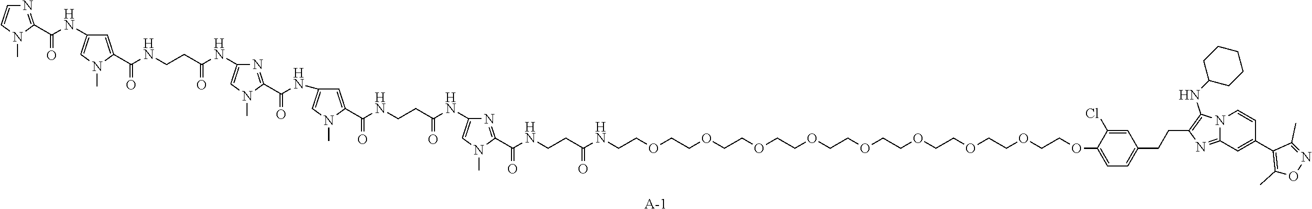 [SMILES: Cc1noc(C)c1-c1ccn2c(NC3CCCCC3)c(CCc3ccc(OCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)c(Cl)c3)nc2c1 ; "A-1"]

Step-1: A mixture of 2-chloro-4-{2-[3-(cyclohexylamino)-7-(3,5-dimethyl-1,2-oxazol-4-yl)imidazo[1,2-a]pyridin-2-yl]ethyl}phenol (150.00 mg, 0.32 mmol, 1.00 equiv) and tert-butyl N-(26-bromo-3,6,9,12,15,18,21,24-octaoxahexacosan-1-yl)carbamate (187.00 mg, 0.32 mmol, 1.01 equiv), K₂CO₃ (134.00 mg, 0.97 mmol, 3.01 equiv) in ACN (5.00 mL) was stirred for 14.0 h at 70.0° C. The resulting mixture was filtered and the filtrate was concentrated under vacuum. The residue was purified by TLC-Plate with DCM/MeOH (10:1) to afford tert-butyl N-[26-(2-chloro-4-{2-[3-(cyclohexylamino)-7-(3,5-dimethyl-1,2-oxazol-4-yl)imidazo[1,2-a]pyridin-2-yl]ethyl}phenoxy)-3,6,9,12,15,18,21,24-octaoxahexacosan-1-yl]carbamate (210.00 mg, 64.46%) as a yellow solid. LC/MS: mass calcd. For C₄₉H₇₄ClN₅On: 959.50, found: 982.80 [M+Na]⁺.

Step 2: A solution of tert-butyl N-[26-(2-chloro-4-{2-[3-(cyclohexylamino)-7-(3,5-dimethyl-1,2-oxazol-4-yl)imidazo[1,2-a]pyridin-2-yl]ethyl}phenoxy)-3,6,9,12,15,18,21,24-octaoxahexacosan-1-yl]carbamate (100.00 mg, 0.10 mmol, 1.00 equiv) and TFA (0.50 mL) in DCM (1.50 mL) was stirred for 1.0 h at room temperature. The resulting mixture was concentrated under reduced pressure. This resulted in 2-(2-{4-[(26-amino-3,6,9,12,15,18,21,24-octaoxahexacosan-1-yl)oxy]-3-chlorophenyl}ethyl)-N-cyclohexyl-7-(3,5-dimethyl-1,2-oxazol-4-yl)imidazo[1,2-a]pyridin-3-amine (100.00 mg, crude) as a yellow oil. LCMS: mass calcd. For C₄₄H₆₆ClN₅O₁₀: 859.45, found: 860.35[M+H]⁺.

Step 3. Synthesis of A-1: A solution of 2-(2-{4-[(26-amino-3,6,9,12,15,18,21,24-octaoxahexacosan-1-yl)oxy]-3-chlorophenyl}ethyl)-N-cyclohexyl-7-(3,5-dimethyl-1,2-oxazol-4-yl)imidazo[1,2-a]pyridin-3-amine (80.00 mg, 0.09 mmol, 1.00 equiv) and 3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanoic acid (77.15 mg, 0.09 mmol, 1.00 equiv), PyBOP (63.38 mg, 0.12 mmol, 1.31 equiv), DIEA (200.00 μL, 1.15 mmol, 12.35 equiv) in DMF (2.00 mL) was stirred for 1.0 h at room temperature. The reaction was poured into ice water (10 mL), and the mixture was stirred for 15 min. The precipitated solids were collected by filtration and washed with water (3×3 mL) and dried under vacuum.

The crude product was purified by Prep-HPLC with the following conditions: Column: YMC-Actus Triart C18 ExRS, 20*250 mm, 5 μm; Mobile Phase A: water (10 mmol/L NH₄HCO₃+0.1% NH₃·H₂O), Mobile Phase B: ACN; Flow rate: 20 mL/min; Gradient: 20% B to 50% B in 8 min, 50% B; Wave Length: 254 nm; RT1 (min): 7.43. The fractions were combined and lyophilized to afford the N-(5-{[2-({2-[(2-{[26-(2-chloro-4-{2-[3-(cyclohexylamino)-7-(3,5-dimethyl-1,2-oxazol-4-yl)imidazo[1,2-a]pyridin-2-yl]ethyl}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formainido}propanamido)imidazole-2-carboxamide (37.80 mg, 23.62%) as a white solid.

HRMS: mass calcd. For C₈₀H₁₀₇ClN₂₀O₁₈: 1670.7761, found: 1671.7821[M+H]⁺.

### Example 7. Synthesis of Compound A-2

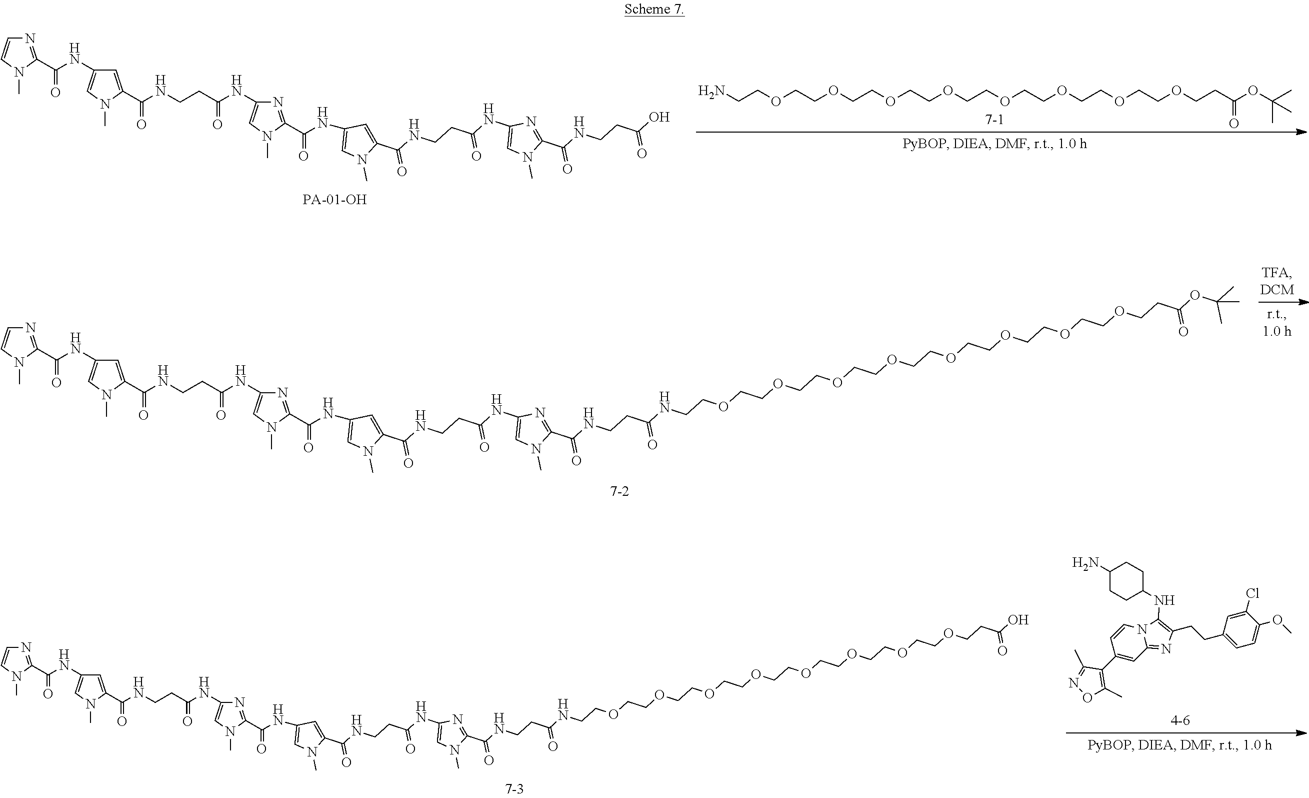 [SMILES: Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)O)n1 ; CC(C)(C)OC(=O)CCOCCOCCOCCOCCOCCOCCOCCOCCN ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOCCOCCC(=O)OC(C)(C)C)n1 ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOCCOCCC(=O)O)n1 ; COc1ccc(CCc2nc3cc(-c4c(C)noc4C)ccn3c2NC2CCC(N)CC2)cc1Cl]

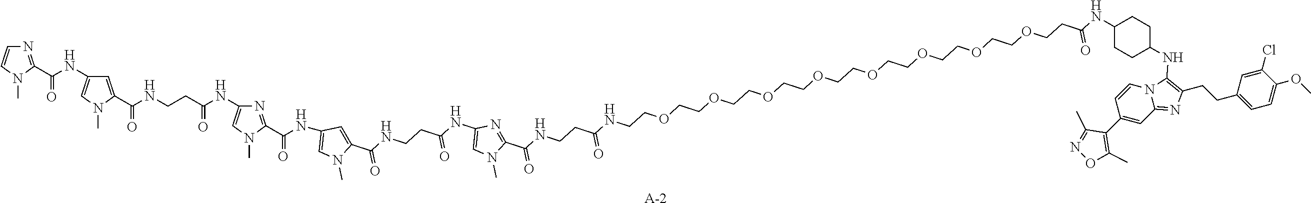 [SMILES: COc1ccc(CCc2nc3cc(-c4c(C)noc4C)ccn3c2NC2CCC(NC(=O)CCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC2)cc1Cl ; "A-2"]

Step 1: A solution of 3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanoic acid (200.00 mg, 0.24 mmol, 1.00 equiv) and tert-butyl 1-amino-3,6,9,12,15,18,21,24-octaoxaheptacosan-27-oate (132.00 mg, 0.26 mmol, 1.10 equiv) and DIEA (94.00 mg, 0.73 mmol, 3.02 equiv) and PyBOP (163.00 mg, 0.31 mmol, 1.30 equiv) in DMF (2.00 mL) was stirred for 1.0 h at room temperature. After that, the reaction was quenched by the addition of water (10 mL). The solid formed was collected by filtration and dried under vacuum to afford tert-butyl 1-[3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanamido]-3,6,9,12,15,18,21,24-octaoxaheptacosan-27-oate (290.00 mg, 91.89%) as a yellow solid. LC/MS: mass calcd. For C₅₉H₈₈N₁₆O₁₈: 1308.65 found: 655.55 [M/2+H]⁺.

Step 2: A solution of tert-butyl 1-[3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanamido]-3,6,9,12,15,18,21,24-octaoxaheptacosan-27-oate (170.00 mg, 0.13 mmol, 1.00 equiv) and TFA (0.30 mL) in DCM (2.00 mL) was stirred for 1.0 h at room temperature. The resulting mixture was concentrated under vacuum. This resulted in 1-[3-({1-methyl-4-[3-({1-methyl-4-[l-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanamido]-3,6,9,12,15,18,21,24-octaoxaheptacosan-27-oic acid (170.00 mg, crude) as a yellow oil. LCMS: mass calcd. For C₅₅H₈₀N₆O₁₈:1252.58, found: 1253.55 [M+H]⁺.

Step 3. Synthesis of A-2: A solution of 1-[3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanamido]-3,6,9,12,15,18,21,24-octaoxaheptacosan-27-oic acid (160.00 mg, 0.13 mmol, 1.00 equiv), N1-{2-[2-(3-chloro-4-methoxyphenyl)ethyl]-7-(3,5-dimethyl-1,2-oxazol-4-yl)imidazo[1,2-a]pyridin-3-yl}cyclohexane-1,4-diamine (63.00 mg, 0.13 mmol, 1.00 equiv), DIEA (82.00 mg, 0.63 mmol, 4.97 equiv) and PyBOP (86.00 mg, 0.16 mmol, 1.29 equiv) in DMF (3.00 mL) was stirred for 1.0 h at room temperature.

The reaction mixture was purified by reverse flash chromatography with the following conditions: Column: C18 silica gel; Mobile Phase: MeCN in water (0.1% TFA), 10% to 50% gradient in 10 min; Detector: UV 254 nm. The fractions were concentrated under vacuum. This resulted in N-{5-[(2-{[2-({2-[(26-{[4-({2-[2-(3-chloro-4-methoxyphenyl)ethyl]-7-(3,5-dimethyl-1,2-oxazol-4-yl)imidazo[1,2-a]pyridin-3-yl}amino)cyclohexyl]carbamoyl}-3,6,9,12,15,18,21,24-octaoxahexacosan-1-yl)carbamoyl]ethyl}carbamoyl)-1-methylimidazol-4-yl]carbamoyl}ethyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide as a yellow solid.

The crude product was purified by Prep-HPLC with the following conditions: Column: YMC-Actus Triart C18 ExRS, 20*250 mm, 5 μm; Mobile Phase A: water (10 mmol/L NH₄HCO₃+0.1% NH₃·H₂O), Mobile Phase B: ACN; Flow rate: 20 mL/min; Gradient: 20% B to 50% B in 8 min, 50% B; Wave Length: 254 nm; RT1 (min): 7.46. The fractions were combined and lyophilized to afford the N-{5-[(2-{[2-({2-[(26-{[4-({2-[2-(3-chloro-4-methoxyphenyl)ethyl]-7-(3,5-dimethyl-1,2-oxazol-4-yl)imidazo[1,2-a]pyridin-3-yl}amino)cyclohexyl]carbamoyl}-3,6,9,12,15,18,21,24-octaoxahexacosan-1-yl)carbamoyl]ethyl}carbamoyl)-1-methylimidazol-4-yl]carbamoyl}ethyl)carbamoyl]-1-methylpyrrol-3-yl}-1-methyl-4-(3-{[1-methyl-4-(1-methylimidazole-2-amido)pyrrol-2-yl]formamido}propanamido)imidazole-2-carboxamide (15.10 mg, 6.70%) as a white solid.

HRMS (ESI): mass calcd. For C₈₂H₁₁₀ClN₂₁O₁₉: 1727.7975, found: 1728.8032 [M+H]⁺.

### Example 8. Synthesis of Compound A-3

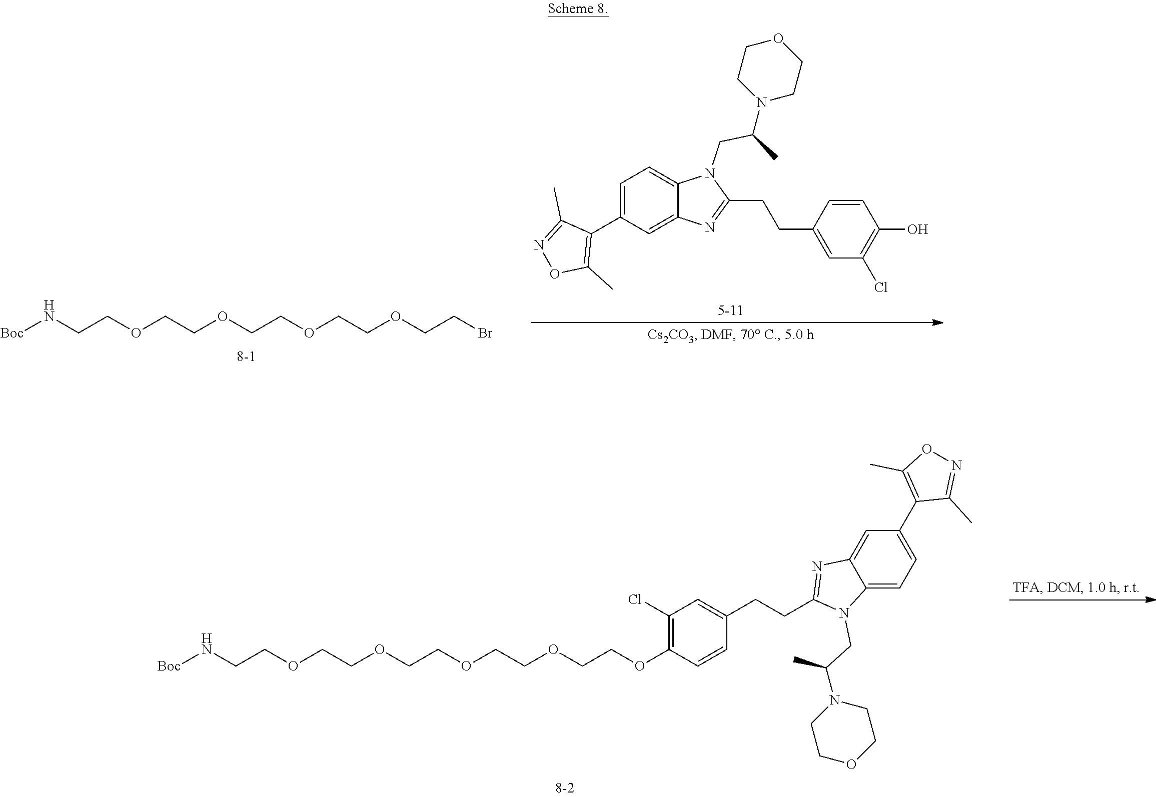 [SMILES: Cc1noc(C)c1-c1ccc2c(c1)nc(CCc1ccc(O)c(Cl)c1)n2C[C@H](C)N1CCOCC1 ; CC(C)(C)OC(=O)NCCOCCOCCOCCOCCBr ; Cc1noc(C)c1-c1ccc2c(c1)nc(CCc1ccc(OCCOCCOCCOCCOCCNC(=O)OC(C)(C)C)c(Cl)c1)n2C[C@H](C)N1CCOCC1]

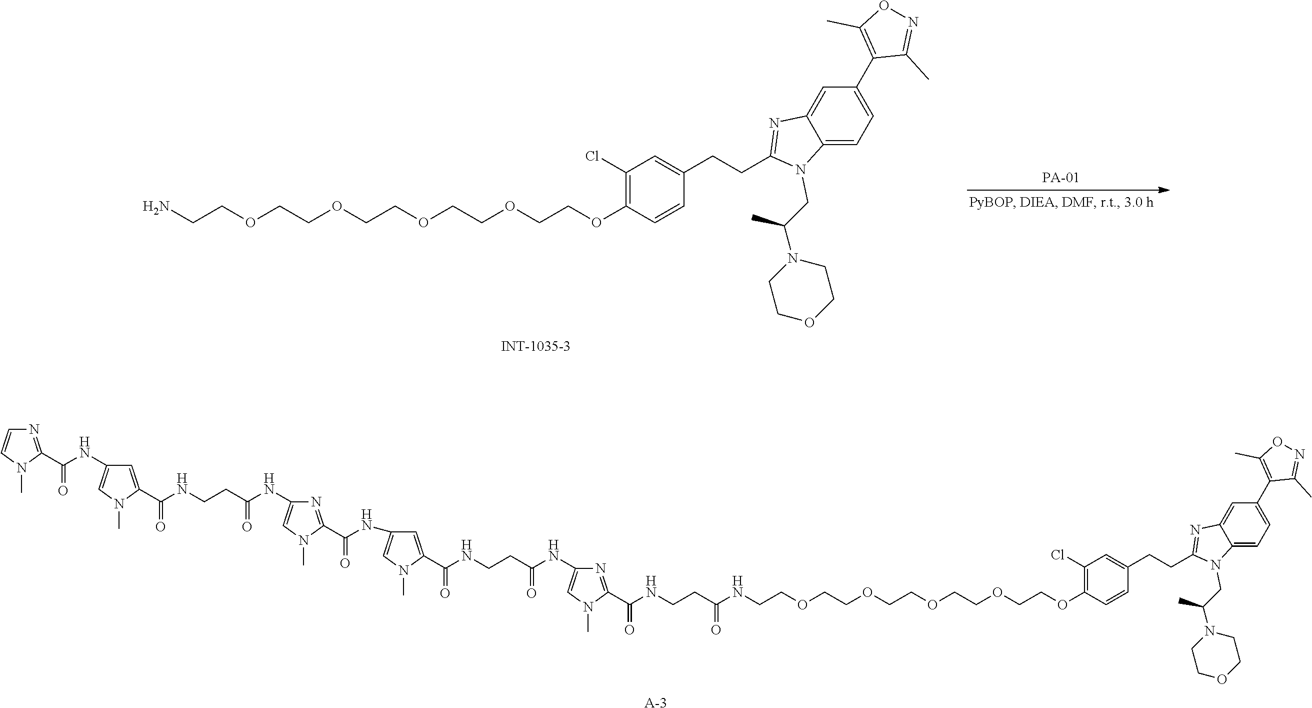 [SMILES: Cc1noc(C)c1-c1ccc2c(c1)nc(CCc1ccc(OCCOCCOCCOCCOCCN)c(Cl)c1)n2C[C@H](C)N1CCOCC1 ; Cc1noc(C)c1-c1ccc2c(c1)nc(CCc1ccc(OCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)c(Cl)c1)n2C[C@H](C)N1CCOCC1 ; "A-3"]

Step 1: To a stirred mixture of tert-butyl N-(14-bromo-3,6,9,12-tetraoxatetradecan-1-yl)carbamate (80.00 mg, 0.20 mmol, 1.00 equiv) and 2-chloro-4-{2-[5-(3,5-dimethyl-1,2-oxazol-4-yl)-1-[(2S)-2-(morpholin-4-yl)propyl]-1,3-benzodiazol-2-yl]ethyl}phenol (118.71 mg, 0.24 mmol, 1.20 equiv) in DMF (4.00 mL) was added Cs₂CO₃ (130.23 mg, 0.40 mmol, 2.00 equiv). The resulting mixture was stirred for 16.0 h at 70° C. The solid was filtered out and the filtrate was concentrated. The residue was purified by reverse flash chromatography with the following conditions: Column: C18 silica gel; Mobile Phase: ACN in water, 10% to 60% gradient in 10 min; Detector: UV 254 nm. The fractions were combined and concentrated to afford tert-butyl N-[14-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12-tetraoxatetradecan-1-yl]carbamate (150.00 mg, 92.16%) as an orange oil. LC/MS: mass calcd. For C₄₂H₆₀ClN₅O₉: 813.41, found: 814.35 [M+H]⁺.

Step 2: To a stirred mixture of tert-butyl N-[14-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12-tetraoxatetradecan-1-yl]carbamate (150.00 mg, 0.18 mmol, 1.00 equiv) in DCM (5.00 mL) was added TFA (1.00 mL) at room temperature. The resulting mixture was stirred for 1.0 h at room temperature. The resulting mixture was concentrated under reduced pressure to afford 14-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12-tetraoxatetradecan-1-amine (150.00 mg, crude) as a yellow oil. LC/MS: mass calcd. For C₃₇H₅₂ClN₅O₇: 713.36, found: 714.35 [M+H]⁺.

Step 3. 6-3. Synthesis of A-3. To a stirred mixture of 14-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12-tetraoxatetradecan-1-amine (100.00 mg, 0.14 mmol, 1.50 equiv), 1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxylic acid (70.77 mg, 0.09 mmol, 1.00 equiv) and EDCI (35.78 mg, 0.19 mmol, 2.00 equiv) in DMF (3.00 mL) was added DMAP (45.61 mg, 0.37 mmol, 4.00 equiv). The resulting mixture was stirred for 16.0 h at room temperature. The reaction mixture was filtered and the filtrate was purified by Prep-HPLC under the conditions: Column; YMC-Actus Triart C18 ExRS, 20*250 mm, 5 m; Mobile Phase A: Water (10 mmol/L NH4HCO₃+0.1% NH₃·H₂O), Mobile Phase B: ACN; Flow rate: 20 mL/min; Gradient: 20% B to 50% B in 8 min, 50% B; Wave Length: 254 nm; RT1 (min): 7.45; Number of Runs: 7). The fractions were combined and lyophilized to afford N-(5-{[2-({2-[(2-{[14-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12-tetraoxatetradecan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (35.60 mg, 24.79%) as a white solid.

HRMS: mass calcd. For C₇₃H₉₃ClN₂₀O₁₅: 1524.6818, found: 1525.6813 [M+H]⁺.

### Example 9. Synthesis of Compound A-4

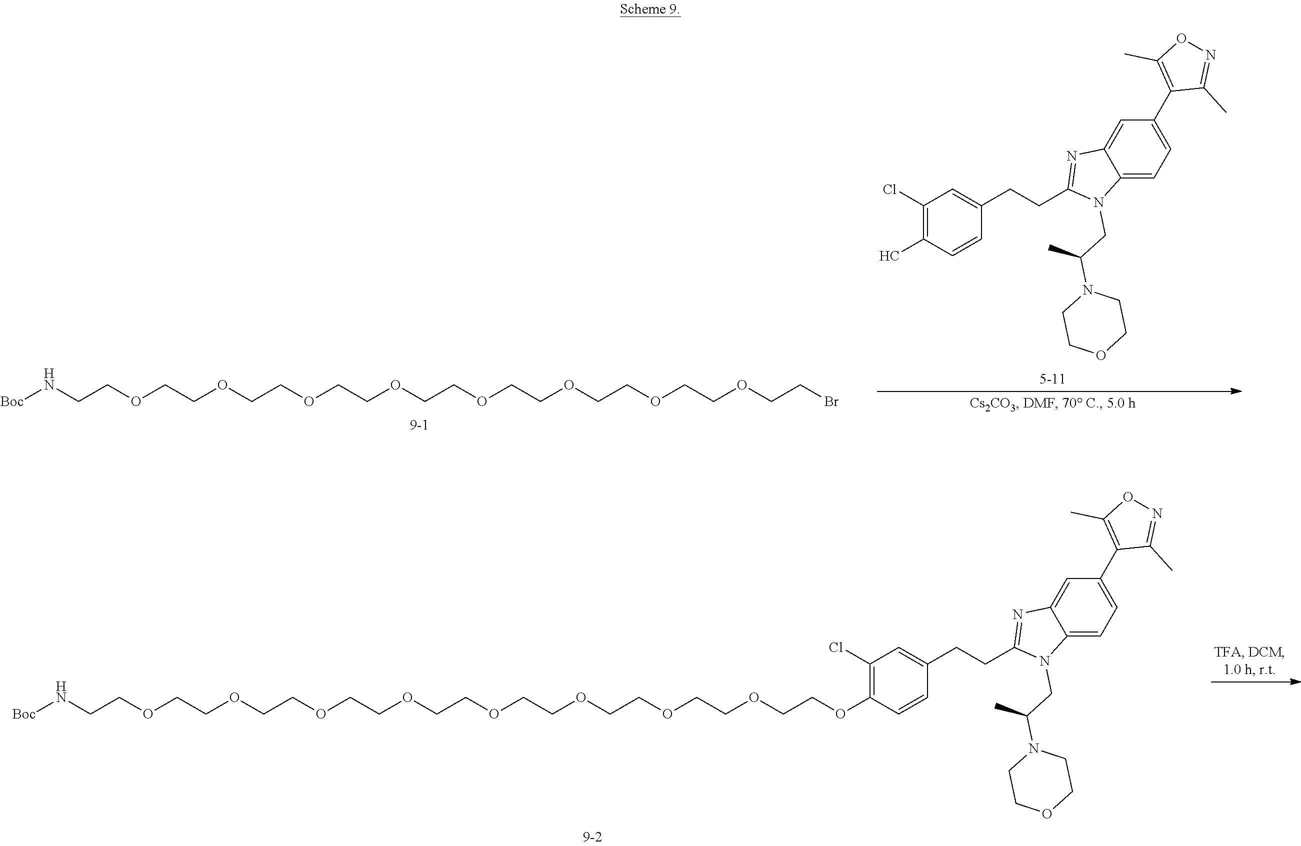

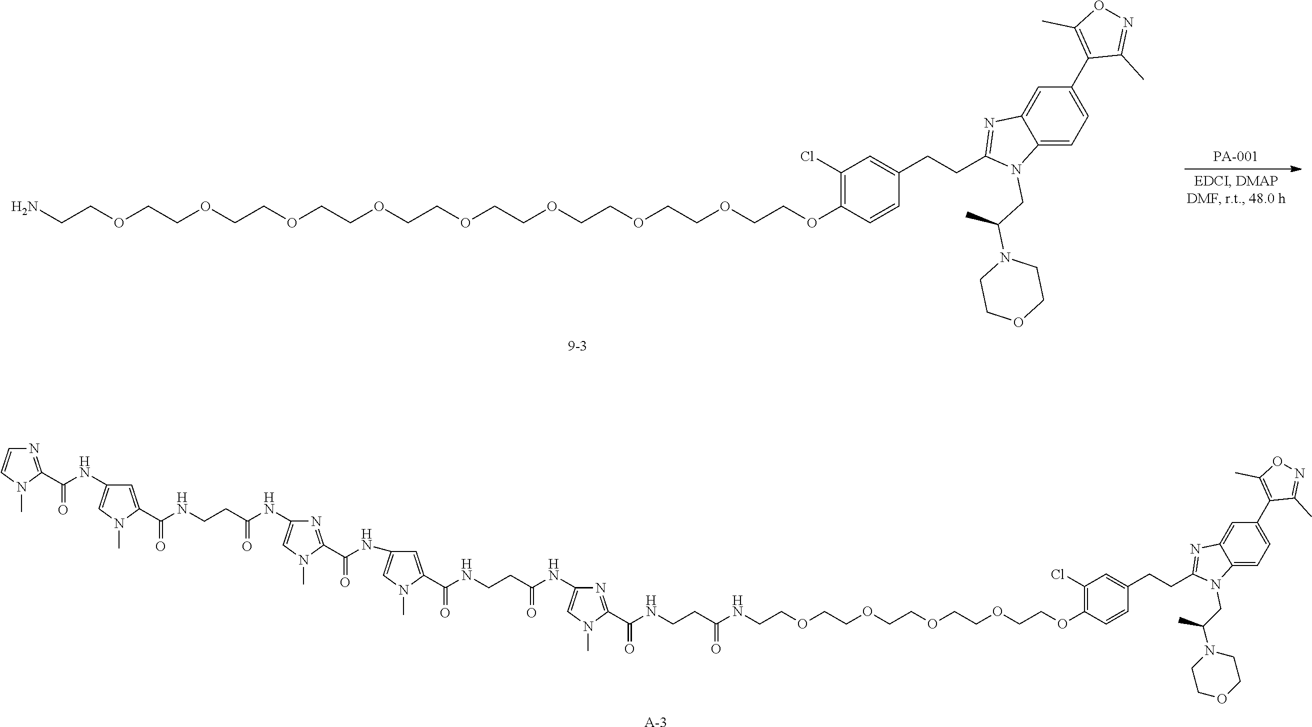

Step 1: To a stirred mixture of tert-butyl N-(26-bromo-3,6,9,12,15,18,21,24-octaoxahexacosan-1-yl)carbamate (80.00 mg, 0.14 mmol, 1.00 equiv) and 2-chloro-4-{2-[5-(3,5-dimethyl-1,2-oxazol-4-yl)-1-[(2S)-2-(morpholin-4-yl)propyl]-1,3-benzodiazol-2-yl]ethyl}phenol (75.56 mg, 0.15 mmol, 1.10 equiv) in DMF (3.00 mL) was added Cs₂CO₃ (90.42 mg, 0.28 mmol, 2.00 equiv). The resulting mixture was stirred for 16.0 h at 70° C. The solid was filtered out and the filtrate was concentrated. The residue was purified by reverse flash chromatography with the following conditions: Column, C18 silica gel; Mobile Phase: ACN in water, 10% to 60% gradient in 10 min; Detector: UV 254 nm. The fractions were combined and concentrated to afford tert-butyl N-[26-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12,15,18,21,24-octaoxahexacosan-1-yl]carbamate (115.00 mg, 83.66%) as orange oil. LC/MS: mass calcd. For C₅₀H₇₀ClN₅O₁₃: 989.51, found: 1012.60 [M+Na]⁺.

Step 2: To a stirred mixture of tert-butyl N-[26-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12,15,18,21,24-octaoxahexacosan-1-yl]carbamate (115.00 mg, 0.12 mmol, 1.00 equiv) in DCM (5.00 mL) was added TFA (1.00 mL) at room temperature. The resulting mixture was stirred for 1.0 h at room temperature. The resulting mixture was concentrated under reduced pressure to afford 26-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12,15,18,21,24-octaoxahexacosan-1-amine (115.00 mg, crude) as a yellow oil. LC/MS: mass calcd. For C₄₅H₆ClN₅O₁₁: 889.46, found: 890.40 [M+H]⁺.

Step 3. Synthesis of A-4: To a stirred mixture of 26-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12,15,18,21,24-octaoxahexacosan-1-amine (100.00 mg, 0.11 mmol, 1.50 equiv), 1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxylic acid (56.60 mg, 0.07 mmol, 1.00 equiv) and EDCI (28.70 mg, 0.15 mmol, 2.00 equiv) in DMF (3.00 mL) was added DMAP (36.58 mg, 0.30 mmol, 4.00 equiv). The resulting mixture was stirred for 16.0 h at room temperature.

The reaction mixture was filtered and the filtration was purified by Prep-HPLC under the conditions: Column: Xselect CSH F-Phenyl OBD column, 19*250 mm, 5 m; Mobile Phase A: Water (0.05% TFA), Mobile Phase B: MeOH—HPLC; Flow rate: 20 mL/min; Gradient: 23% B to 50% B in 8 min, 50% B; Wave Length: 254 nm; RT1 (min): 7; Number of Runs: 6). The fractions were combined and lyophilized to afford N-(5-{[2-({2-[(2-{[26-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (38.40 mg, 29.42%) as a yellow solid.

HRMS: mass calcd. For C₈₁H₁₀₉ClN₂₀O₁₉: 1700.7866, found: 1701.7849 [M+H]⁺.

### Example 10. Synthesis of Compound A-5

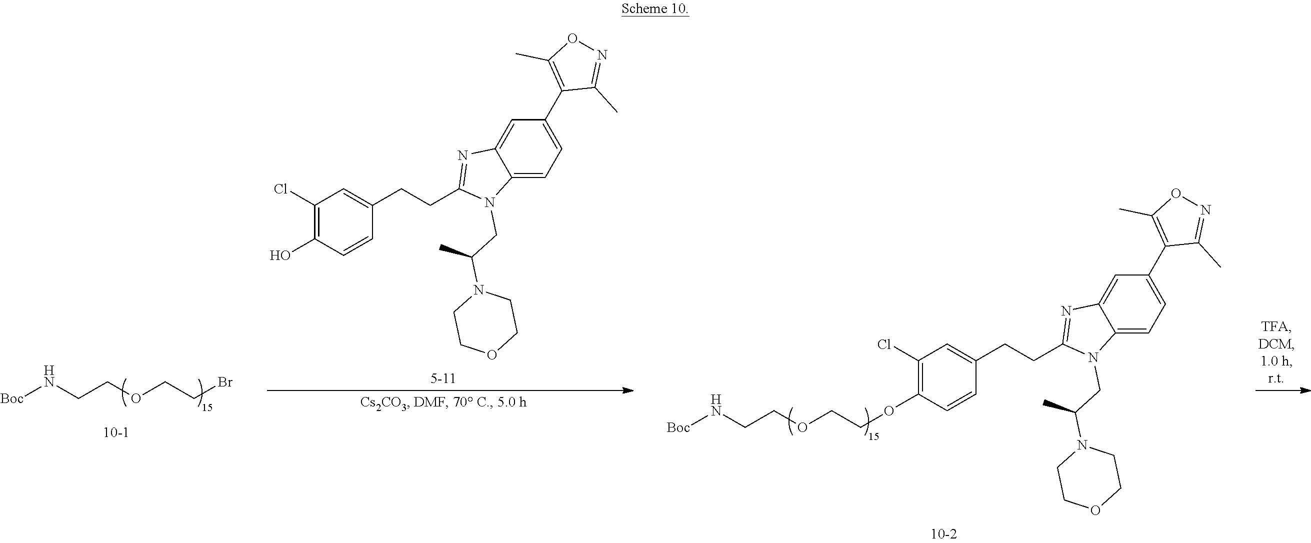

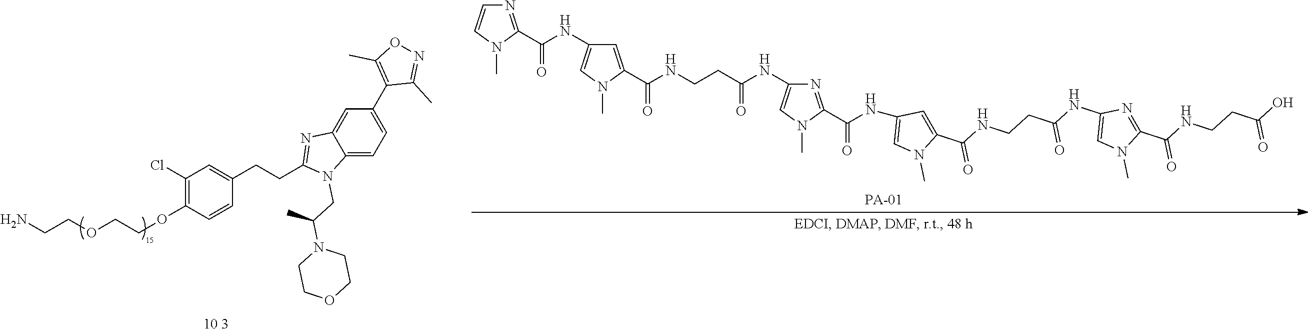

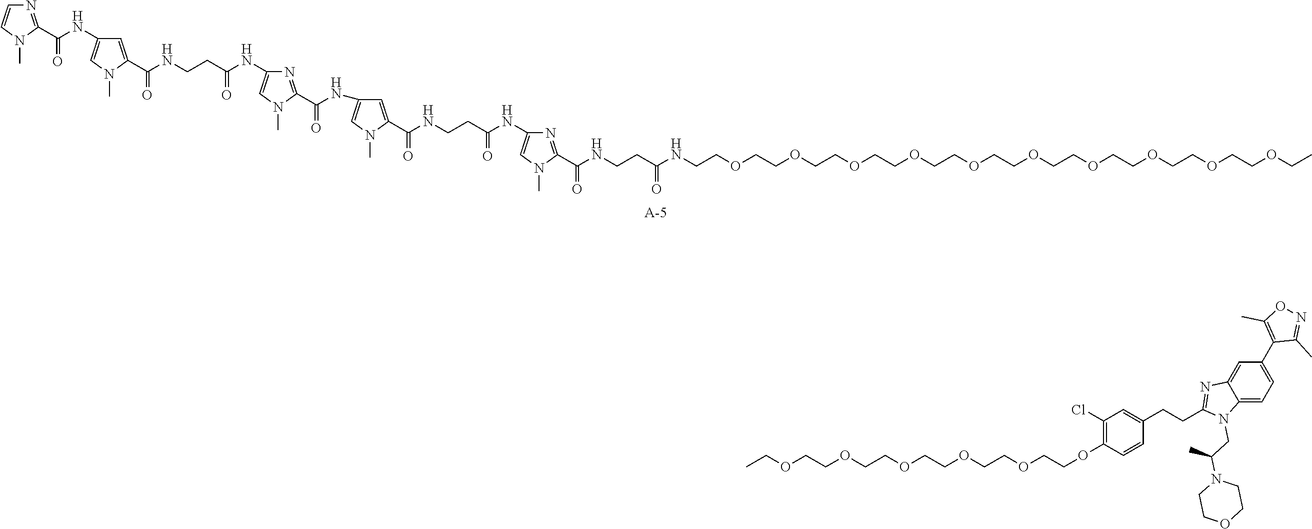

Step 1: To a stirred mixture of tert-butyl N-(47-bromo-3,6,9,12,15,18,21,24, 27, 30, 33, 36, 39, 42,45-pentadecaoxaheptatetracontan-1-yl)carbamate (80.00 mg, 0.09 mmol, 1.00 equiv) and 2-chloro-4-{2-[5-(3,5-dimethyl-1,2-oxazol-4-yl)-1-[(2S)-2-(morpholin-4-yl)propyl]-1,3-benzodiazol-2-yl]ethyl}phenol (49.23 mg, 0.10 mmol, 1.10 equiv) in DMF (3.00 mL) was added Cs₂CO₃ (58.91 mg, 0.18 mmol, 2.00 equiv). The resulting mixture was stirred for 16.0 h at 70° C. The solid was filtered out and the filtrate was concentrated. The residue was purified by reverse flash chromatography with the following conditions: Column, C18 silica gel; Mobile Phase: ACN in water, 10% to 60% gradient in 10 min; Detector, UV 254 nm. The fractions were combined and concentrated to afford tert-butyl N-[47-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12,15,18,21,24, 27, 30, 33, 36, 39, 42,45-pentadecaoxaheptatetracontan-1-yl]carbamate (120.00 mg, quantitative) as an orange oil. LC/MS: mass calcd. For C₆₄H₁₀₄ClN₅O₂₀:1297.70, found: 1298.65 [M+H]⁺.

Step 2: To a stirred mixture of tert-butyl N-[47-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12,15,18,21,24, 27, 30, 33, 36, 39, 42,45-pentadecaoxaheptatetracontan-1-yl]carbamate (120.00 mg, 0.09 mmol, 1.00 equiv) in DCM (5.00 mL) was added TFA (1.00 mL) at room temperature.

The resulting mixture was stirred for 1.0 h at room temperature. The resulting mixture was concentrated under reduced pressure to afford 47-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12,15,18,21,24, 27, 30, 33, 36, 39, 42,45-pentadecaoxaheptatetracontan-1-amine (120.00 mg, crude) as a yellow oil.LC/MS: mass calcd. For C₅₉H₉₆ClN₅O₁₃: 1197.64, found: 1198.60 [M+H]⁺.

Step 3. Synthesis of A-5: To a stirred mixture of 47-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12,15,18,21,24, 27, 30, 33, 36, 39, 42,45-pentadecaoxaheptatetracontan-1-amine (115.00 mg, 0.10 mmol, 1.50 equiv), 3-({1-methyl-4-[3-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)propanamido]imidazol-2-yl}formamido)propanoic acid (53.10 mg, 0.06 mmol, 1.00 equiv) and EDCI (18.34 mg, 0.10 mmol, 1.50 equiv) in DMF (3.00 mL) was added DMAP (19.53 mg, 0.16 mmol, 2.50 equiv). The resulting mixture was stirred for 16.0 h at room temperature. The reaction mixture was filtered and the filtration was purified by Prep-HPLC under the conditions: Column: XBridge Prep Phenyl OBD Column, 19*250 mm, 5 μm; Mobile Phase A: Water (0.05% TFA), Mobile Phase B: MeOH-HPLC; Flow rate: 25 mL/min; Gradient: 56% B to 80% B in 10 min, 80% B to 80% B in 16 min, 80% B; Wave Length: 254 nm; RT1 (min): 12.9; Number of Runs: 2). The fractions were combined and lyophilized to afford N-(5-{[2-({2-[(2-{[47-(2-chloro-4-{2-[5-(3,5-dimethyl-1,2-oxazol-4-yl)-1-[(2S)-2-(morpholin-4-yl)propyl]-1,3-benzodiazol-2-yl]ethyl}phenoxy)-3,6,9,12,15,18,21,24,27,30,33, 36,39,42,45-pentadecaoxaheptatetracontan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (15.40 mg, 11.97%) as a white solid.

HRMS: mass calcd. For C₉₅H₃₇ClN₂₀O₂₆: 2008.9701, found: 2009.9720 [M+H]⁺.

### BIOLOGICAL EXAMPLES

### Example B1: EC₅₀ Assay

Cell culture: Cells are cultured in RPMI1640 medium+15% FBS. Cells are maintained at a density between 2×10⁶/mL and 1×10⁶/mL. Cells are centrifuged, resuspended in fresh medium, counted and plated at 150,000 cells per well in 100 μL in a non-coated, flat bottom tissue culture plate.

Compound treatment: 10 mM stock solution of FA transcription modulator compounds is diluted 1:10 in DMSO followed by a 1:100 dilution in growth medium. Working solution is then further diluted to 10× desired final concentration of 150 nM. Compound are then diluted at a 1:3 ratio into growth medium containing 0.01% DMSO. 5-point, 3-fold dose response curve is generated. 11 μL of 10× compound is added to wells containing 100 μL cell suspension of GM15850. 11 μL growth medium containing 0.01% DMSO is added to all wells not treated with FA GeneTAC™. Cells are allowed to incubate for 48 hrs prior to cell lysis using guanidine isothiocyanate solution.

RNA isolation: Total RNA is isolated and purified in 384-well column filter plates using chaotropic salt.

qRT-PCR: qRT-PCR reactions are assembled using AgPath-ID reagents (Thermo Fisher) using 6 L mastermix and 4 μL RNA. qRT-PCR TaqMan primer probe sets against human FXN (Assay ID Hs01075496_ml) and human GAPDH (Assay ID Hs00266705_gl) are used to measure the intended targets. qRT-PCR is run on the ThermoFisher QuantStudio 6 PRO instrument using the manufacturer's recommended cycling conditions.

Data analysis: qPCR data is analyzed using Thermo Fisher Design and Analysis software. Data is exported to Excel and hFXN expression is nonnalized to hGAPDH expression.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A transcription modulator molecule having a first terminus, a second terminus, and a linker moiety, wherein:
(a) the first terminus comprises a DNA-binding moiety capable of noncovalently binding to a nucleotide repeat sequence comprising GAA;
(b) the second terminus comprises a protein-binding moiety binding to a regulatory molecule that modulates an expression of a gene comprising the nucleotide repeat sequence GAA; and
(c) the linker comprising an oligomeric backbone that connects the first terminus and the second terminus; and
wherein the first terminus has the structure of Formula (A-1), or a pharmaceutically acceptable salt thereof: 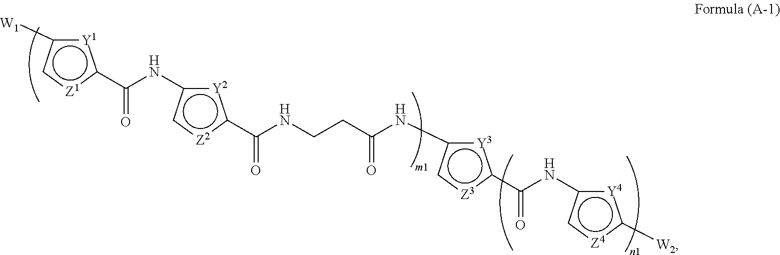
wherein;
m₁ is 1-4;
n₁ is 0-2;
each Y¹, Y², Y³, and Y⁴ is independently CH or N;
each Z¹, Z², Z³, and Z⁴ is independently O, S, or NR²;
W₁ is hydrogen, deuterium, halogen, optionally substituted C₁-C₁₀ alkyl, —NR^{1c}C(O)NR^{1e}R^{1f}, —C(O)NR^{1e}R^{1f}, —O—C(O)NR^{1e}R^{1f}, —NR^{1e}C(O)—OR^{1f}, or (AA)₁₋₁₀;
W₂ is hydrogen, deuterium, halogen, optionally substituted C₁-C₁₀ alkyl, —C(O)NR^{1e}R^{1f}, or (AA)₁₋₁₀; wherein
each R^{1e} is independently hydrogen, optionally substituted C₁-C₅₀ alkyl, optionally substituted C₁-C₅₀ alkenyl, optionally substituted C₁-C₅₀ alkynyl, optionally substituted C₁-C₅₀ heteroalkyl, optionally substituted C₁-C₅₀ heteroalkenyl, optionally substituted C₁-C₅₀ heteroalkynyl or PEG₁₋₅₀;
each R^{1f} is independently hydrogen, optionally substituted C₁-C₂₀ alkyl, optionally substituted C₁-C₂₀ alkenyl, optionally substituted C₁-C₂₀ alkynyl, optionally substituted C₁-C₂₀ heteroalkyl, optionally substituted C₁-C₂₀ heteroalkenyl, optionally substituted C₁-C₂₀ heteroalkynyl, PEG₁₋₂₀, or one or more AA, wherein each AA is independently a naturally occurring amino acid;
or R^{1e} and R^{1f} can combine together with the nitrogen which they are attached to form an optionally substituted heterocycloalkyl; and
each R² is independently hydrogen, optionally substituted C₁-C₅₀ alkyl, optionally substituted C₁-C₅₀ alkenyl, optionally substituted C₁-C₅₀ alkynyl, optionally substituted C₁-C₅₀ heteroalkyl, optionally substituted C₁-C₅₀ heteroalkyl, optionally substituted C₁-C₅₀ heteroalkenyl, optionally substituted C₁-C₅₀ heteroalkynyl, optionally substituted C₃-C₈ cycloalkyl or optionally substituted 3 to 8-membered heterocycloalkyl, or PEG₁₋₅₀.

2. The molecule of claim 1, or a pharmaceutically acceptable salt thereof, wherein W₂ is —C(O)NR¹OR^{1f}.

3. The molecule of claim 2, or a pharmaceutically acceptable salt thereof, wherein W₂ is —C(O)NH(CH₂)₂C(O)—.

4. The molecule of any one of claims 1-3, wherein the first terminus comprises a structure of Formula (A-2), or a pharmaceutically acceptable salt thereof:

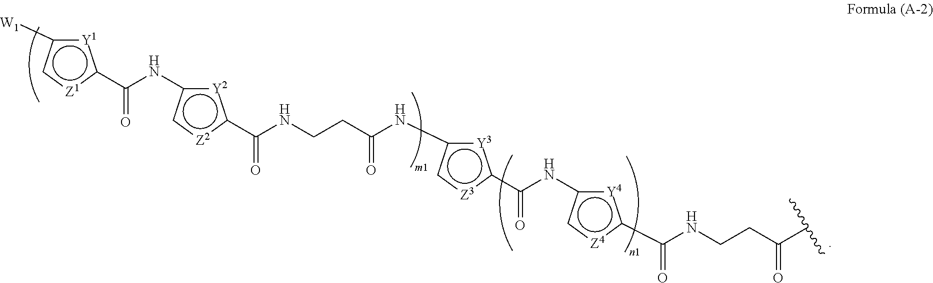

5. The molecule of any one of claims 1-4, or a pharmaceutically acceptable salt thereof, wherein each Z¹, Z², Z³, and Z⁴ is independently NR², wherein each R² is independently an optionally substituted C₁₋₆ alkyl.

6. The molecule of claim 5, or a pharmaceutically acceptable salt thereof, wherein each Z¹, Z², Z³, and Z⁴ is independently NCH₃.

7. The molecule of claim 4, or a pharmaceutically acceptable salt thereof, wherein the first terminus comprises a structure of Formula (A-3), or a pharmaceutically acceptable salt thereof:

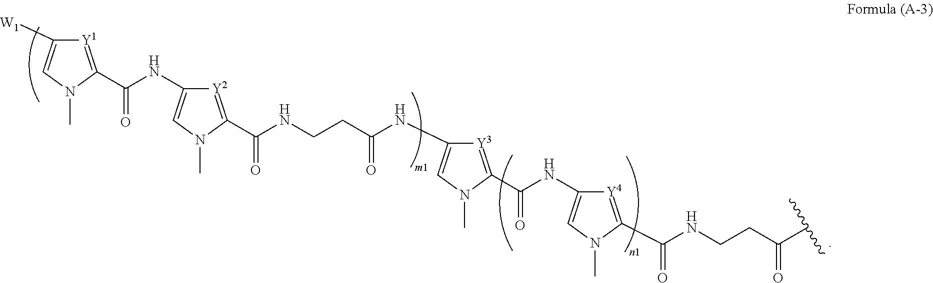

8. The molecule of any one of claims 1-7, or a pharmaceutically acceptable salt thereof, wherein each Y¹ and Y³ are N; and each Y² and Y⁴ are independently CH or N.

9. The molecule of claim 8, or a pharmaceutically acceptable salt thereof, wherein each Y² and Y⁴ are each CH.

10. The molecule of any one of claims 1-9, or a pharmaceutically acceptable salt thereof, wherein W₁ is hydrogen.

11. The molecule of any one of claims 1-10, wherein m₁ is 2 or 3; and n₁ is 0 or 1.

12. The modulator molecule of any one of claims 1-11, or a pharmaceutically acceptable salt thereof, wherein the first terminus is capable of binding the DNA with an affinity of less than 500 nM.

13. The molecule of any one of claims 1-12, or a pharmaceutically acceptable salt thereof, wherein the linker has a length of less than about 50 Angstroms.

14. The molecule of any one of claims 1-12, or a pharmaceutically acceptable salt or solvate thereof, wherein the oligomeric backbone is a linker having a length of about 10 to 60 Angstroms.

15. The molecule of any one of claims 1-12, or a pharmaceutically acceptable salt thereof, wherein the linker comprises between 5 and 50 chain atoms.

16. The molecule of any one of claims 1-15, or a pharmaceutically acceptable salt thereof, wherein the linker comprises a multimer having from 2 to 50 spacing moieties, wherein
each spacing moiety is independently selected from the group consisting of —((CR^{3a}R^{3b})ₓ—O)_{y}—, —((CR^{3a}R^{3b})ₓ—NR^{4a})_{y}—, —((CR^{3a}R^{3b})ₓ—CH═CH—(CR^{3a}R^{3b})ₓ—O)_{y}—, optionally substituted C₁-C₁₂ alkyl, optionally substituted C₂-C₁₀ alkenyl, optionally substituted C₂-C₁₀ alkynyl, optionally substituted C₆-C₁₀ arylene, optionally substituted C₃-C₇ cycloalkylene, optionally substituted 5 to 10-membered heteroarylene, optionally substituted 4 to 10-membered heterocycloalkylene, amino acid residue, —O—, —C(O)NR^{1a}—, —NR^{1a}C(O)—, —C(O)—, —NR^{1a}—, —C(O)O—, —S—, —S(O)—, —S(O)₂—, —S(O)₂NR^{1a}—, —NR^{1a}S(O)₂—, and —P(O)OH—, and any combinations thereof; wherein
each x is independently 2-4;
each y is independently 1-10;
each R^{1a} is independently a hydrogen or optionally substituted C₁-C₆ alkyl;
each R^{3a} and R^{3b} is independently selected from hydrogen, optionally substituted alkyl, optionally substituted alkenyl, optionally substituted alkynyl, optionally substituted alkoxy, optionally substituted amino, carboxyl, carboxyl ester, acyl, acyloxy, acyl amino, amino acyl, optionally substituted alkylamide, sulfonyl, optionally substituted thioalkoxy, optionally substituted aryl, optionally substituted heteroaryl, optionally substituted cycloalkyl, and optionally substituted heterocyclyl; and
each R^{4a} is independently a hydrogen or an optionally substituted C₁-C₆ alkyl.

17. The transcription modulator molecule of claim 16, or a pharmaceutically acceptable salt thereof, wherein each spacing moiety is independently selected from the group consisting of —((CH₂)ₓ—O)_{y}—, —((CH₂)ₓ—NH)_{y}—, —O—, —C(O) NH—, and —NH—, and any combinations thereof.

18. The transcription modulator molecule of any one of claims 1-15, or a pharmaceutically acceptable salt thereof, wherein the oligomeric backbone comprises —(CH₂CH₂—O)_{y}—, —(CH₂CH₂—O)_{y}—(CH₂CH₂)—NH—, —NH—(CH₂CH₂—O)_{y}—, —NH—(CH₂CH₂—O)_{y}—(CH₂CH₂)—NH—, —(CH₂CH₂—O)_{y}—(CH₂CH₂)—NHC(O)—, or —NH—(CH₂CH₂—O)_{y}—(CH₂CH₂)—NHC(O)—, wherein y is 1-50.

19. The molecule of any one of claims 1-18, or a pharmaceutically acceptable salt thereof, wherein the second terminus comprises a moiety that binds to a bromodomain protein.

20. The molecule of claim 19, or a pharmaceutically acceptable salt thereof, wherein the bromodomain protein is a BET bromodomain protein.

21. The molecule of claim 19, or a pharmaceutically acceptable salt thereof, wherein the bromodomain protein is a non-BET bromodomain protein.

22. The molecule of any one of claims 19-21, or a pharmaceutically acceptable salt thereof, wherein the bromodomain protein is not bromodomain 4 (BRD4).

23. The molecule of any one of claims 1-18, or a pharmaceutically acceptable salt thereof, wherein the second terminus is not a bromodomain 4 (BRD4) ligand

24. The molecule of any one of claims 1-18, or a pharmaceutically acceptable salt thereof, wherein the second terminus comprises a bromodomain binding moiety, selected from CBP/p300, PCAF (P300/CBP-Associated Factor), CECR2 (cat eye syndrome chromosome region candidate 2), BRPF (bromodomain and PHD finger-containing protein), ATAD2/ATAD2B (chromatin remodeling proteins), TRIM24 (Tripartite motif-containing 24), BAZ2 (Bromodomain Adjacent to Zinc finger), TAF1 (TBP associated factors), BRD 8 (bromodomain-containing protein 8), and BRD 7/9 (bromodomain-containing protein 7, 9).

25. The molecule of any one of claims 1-18, wherein the second terminus comprises Formula (2-A), or a pharmaceutically acceptable salt thereof: 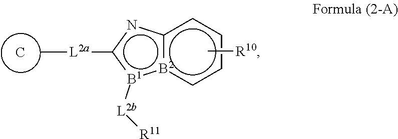
wherein;
ring C is absent, optionally substituted 6-membered aryl, or optionally substituted 6-membered heteroaryl;
B¹ and B² are each independently C or N, wherein one of B¹ or B² is N;
L^{2a} and L²b are each independently absent, optionally substituted alkylene, —O—, or —NR^{12a}—, wherein R^{12a} is hydrogen, deuterium, or optionally substituted C₁-C₁₀ alkyl;
R¹⁰ is optionally substituted 5 to 6-membered heteroaryl;
R¹¹ is hydrogen, optionally substituted C₃-C₈ cycloalkyl, or optionally substituted 3 to 8-membered heterocycloalkyl; and
wherein Formula (2-A) is connected to the linker through ring C or through R¹¹.

26. The molecule of any one of claims 1-18, wherein the second terminus comprises Formula (3-A), or a pharmaceutically acceptable salt thereof: 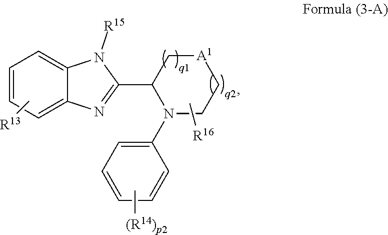
wherein;
A¹ is —CR¹⁷R¹⁷— or —NR¹⁷—, wherein
R¹⁷ is hydrogen or an optionally substituted C₁-C₆ alkyl;
R¹³ is an optionally substituted 5 to 6-membered heteroaryl;
each R¹⁴ is independently hydrogen, halogen, C₁-C₆ alkyl, or C₁-C₆ haloalkyl;
R¹⁵ is an optionally substituted C₁-C₁₀ alkyl, C₃-C₈ cycloalkyl, or 3 to 8-membered heterocycloalkyl;
R¹⁶ is hydrogen, halogen, —OH, —CN, —NO₂, —NH₂, oxo (═O), ═S, C₁-C₁₀ haloalkyl, or C₁-C₁₀ hydroxyalkyl;
p₂ is 1-4;
q₁ and q₂ are each independently 0-2; and
wherein Formula (3-A) is connected to the linker through R¹⁵ or through R¹⁷.

27. The molecule of any one of claims 1-18, wherein the second terminus comprises Formula (4-A), or a pharmaceutically acceptable salt thereof: 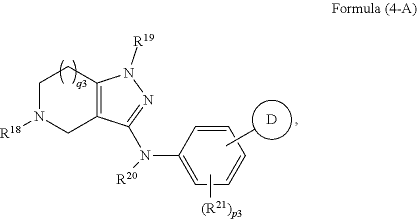
wherein;
ring D is absent or optionally substituted 5 to 6-membered heteroaryl;
R¹⁸ is optionally substituted C₁-C₆ alkyl, optionally substituted C₃-C₈ cycloalkyl, —C(O) R^{18a}, or —C(O)—NR^{18a}R^{18b}, wherein
R^{18a} and R^{18b} are each independently optionally substituted C₁-C₁₀ alkyl or optionally substituted C₃-C₈ cycloalkyl;
R¹⁹ is optionally substituted C₁-C₁₀ alkyl, optionally substituted C₁-C₁₀ haloalkyl, optionally substituted C₃-C₈ cycloalkyl, or optionally substituted 3 to 8 membered heterocycloalkyl;
R²⁰ is hydrogen or optionally substituted C₁-C₁₀ alkyl;
each R²¹ is independently hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, optionally substituted C₂-C₁₀ alkynyl, optionally substituted C₃-C₈-cycloalkyl, or optionally substituted 3- to 8-membered heterocycle;
or R²⁰ and one of R²¹ together with the atoms to which they are attached form an optionally substituted 5 to 8-membered heterocycloalkyl;
p₃ is 1-4;
q₃ is 0 or 1; and
wherein Formula (4-A) is connected to the linker through ring D or through R¹⁸.

28. The molecule of any one of claims 1-18, wherein the second terminus comprises Formula (5-A), or a pharmaceutically acceptable salt thereof: 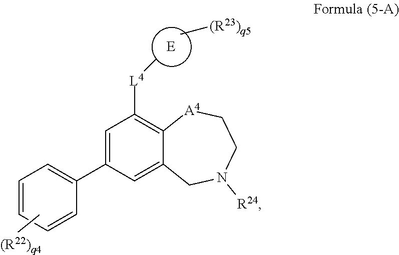
wherein;
ring E is a 5 to 6-membered heterocycloalkyl;
A⁴ is absent, CH₂, —NH—, or —O—;
L⁴ is alkylene or heteroalkylene;
each R²² is independently halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, optionally substituted C₂-C₁₀ alkynyl, optionally substituted C₃-C₈ cycloalkyl, or optionally substituted 3 to 8-membered heterocycloalkyl;
each R²³ is independently hydrogen, halogen, —OH, —CN, —NO₂, —NH₂, optionally substituted C₁-C₁₀ alkyl, optionally substituted C₁-C₁₀ haloalkyl, —O—C₁-C₁₀ alkyl, optionally substituted C₂-C₁₀ alkenyl, optionally substituted C₂-C₁₀ alkynyl, optionally substituted C₃-C₈-cycloalkyl, or optionally substituted 3 to 8-membered heterocycloalkyl;
R²⁴ is optionally substituted C₁-C₁₀ alkyl, —C(O) R^{24a}, or —C(O)—NR^{24a}R^{24b}, wherein
R^{24a} and R^{24b} are each independently optionally substituted C₁-C₁₀ alkyl or optionally substituted C₃-C₈ cycloalkyl;
q₄ is 2-3;
q₅ is 0-2; and
wherein the Formula (5-A) is connected to the linker through ring E or through one of R²².

29. The molecule of any one of claims 1-18, wherein the second terminus comprises Formula (6-A), or a pharmaceutically acceptable salt thereof: 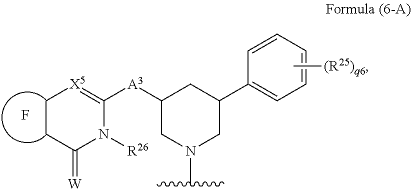
wherein;
ring F is optionally substituted 5 to 6-membered heteroaryl;
A³ is —O—, —NH—, or —CH₂—;
X⁵ is CH or N;
W is O or S;
each R²⁵ is independently hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, optionally substituted C₂-C₁₀ alkynyl, optionally substituted C₃-C₈-cycloalkyl, or optionally substituted 3 to 8-membered heterocycloalkyl;
or two R²⁵ together with the atoms to which they are attached form an optionally substituted C₅-C₈ cycloalkyl or optionally substituted 5 to 8-membered heterocycloalkyl;
R²⁶ is hydrogen or optionally substituted C₁-C₁₀ alkyl; and
q₆ is 1-4.

30. The molecule of any one of claims 1-18, wherein the second terminus comprises Formula (6-B), or a pharmaceutically acceptable salt thereof: 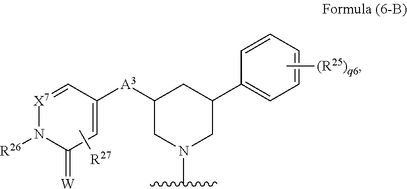
wherein;
A³ is —O—, —NH—, or —CH₂—;
X⁷ is CH or N;
W is O or S;
each R²⁵ is independently hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, optionally substituted C₂-C₁₀ alkynyl, optionally substituted C₃-C₈-cycloalkyl, or optionally substituted 3 to 8-membered heterocycloalkyl;
or two R²⁵ together with the atoms to which they are attached form an optionally substituted C₅-C₈ cycloalkyl or optionally substituted 5 to 8-membered heterocycloalkyl;
R²⁶ is hydrogen or optionally substituted C₁₋₁₀ alkyl;
R²⁷ is hydrogen, halogen, —OH, —CN, —NO₂, —NH₂, optionally substituted C₁-C₁₀ alkyl, optionally substituted C₁-C₁₀ haloalkyl, or optionally substituted C₁-C₁₀ hydroxyalkyl; and
q₆ is 1-4.

31. The molecule of any one of claims 1-18, wherein the second terminus comprises Formula (7-A), or a pharmaceutically acceptable salt thereof: 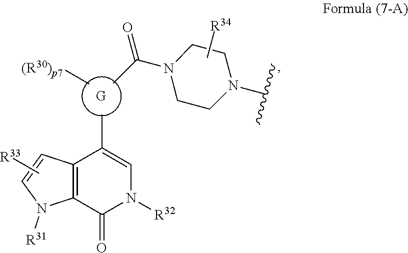
wherein;
ring G is an aryl or heteroaryl;
each R³⁰ is independently hydrogen, halogen, —OH, —CN, —NO₂, —NH₂, C₁-C₁₀ alkyl, C₁-C₁₀ haloalkyl, or C₁-C₁₀ hydroxyalkyl;
R³¹ and R³² are each independently hydrogen, optionally substituted C₁-C₁₀ alkyl, optionally substituted C₁-C₁₀ haloalkyl, optionally substituted C₂-C₁₀ alkenyl, or optionally substituted C₂-C₁₀ alkynyl;
R³³ is hydrogen, halogen, —OH, —CN, —NO₂, —NH₂, optionally substituted C₁-C₁₀ alkyl, optionally substituted C₁-C₁₀ haloalkyl, optionally substituted C₁-C₁₀ hydroxyalkyl, optionally substituted C₂-C₁₀ alkenyl, or optionally substituted C₂-C₁₀ alkynyl;
R³⁴ is hydrogen, halogen, —OH, —CN, —NO₂, —NH₂, optionally substituted C₁-C₁₀ alkyl, optionally substituted C₁-C₁₀ haloalkyl, or optionally substituted C₁-C₁₀ hydroxyalkyl; and
p₇ is 1-4.

32. The molecule of claim 31, or a pharmaceutically acceptable salt thereof, wherein ring G is phenyl.

33. The molecule of claim 31 or 32, wherein the second terminus comprises Formula (7-B): 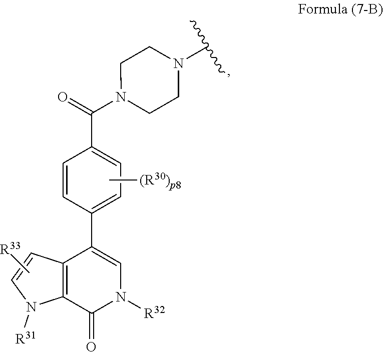
wherein; p₈ is 1-3.

34. The molecule of claim 31, or a pharmaceutically acceptable salt thereof, wherein ring G is a bicyclic heteroaryl comprising 1-2 heteroatoms selected from N, O, or S.

35. The molecule of claim 31 or 32, wherein the second terminus comprises Formula (7-C), or a pharmaceutically acceptable salt thereof: 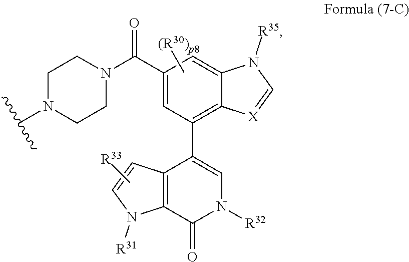
wherein;
X is CR³⁰ or N;
R³⁵ is hydrogen or optionally substituted C₁-C₁₀ alkyl; and
p₈ is 1-3.

36. The molecule of any one of claims 1-18, wherein the second terminus comprises Formula (8-A), or a pharmaceutically acceptable salt thereof: 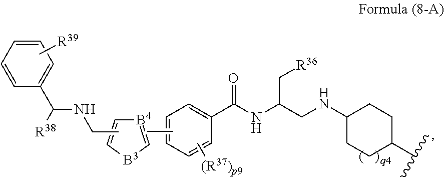
wherein;
B³ is —O—, —NH—, or S;
B⁴ is N or CH;
R³⁶ is an optionally substituted aryl or optionally substituted heteroaryl;
each R³⁷ is independently halogen, —OH, —CN, —NO₂, —NH₂, optionally substituted C₁-C₁₀ alkyl, -optionally substituted C₁-C₁₀ haloalkyl, or optionally substituted C₁-C₁₀ hydroxyalkyl;
R³⁸ is hydrogen, optionally substituted C₁-C₁₀ alkyl, optionally substituted C₁-C₁₀ haloalkyl, or optionally substituted C₁-C₁₀ hydroxyalkyl;
R³⁹ is halogen, —OH, —CN, —NO₂, —NH₂, optionally substituted C₁-C₁₀ alkyl, optionally substituted C₁-C₁₀ haloalkyl, or optionally substituted C₁-C₁₀ hydroxyalkyl;
p₉ is 1-3; and
q₄ is 0-2.

37. The molecule of any one of claims 1-18, or a pharmaceutically acceptable salt thereof, wherein the second terminus is: 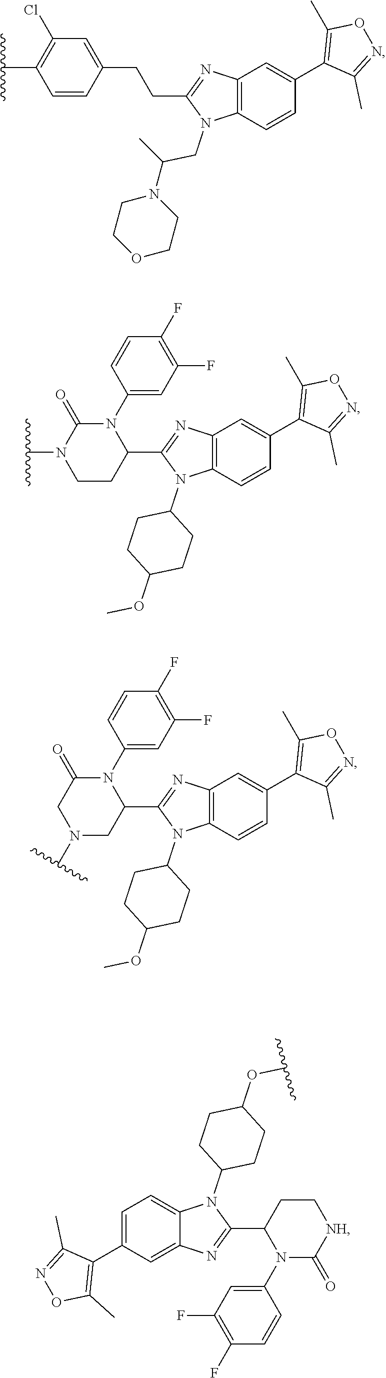 [SMILES: *c1ccc(CCc2nc3cc(-c4c(C)noc4C)ccc3n2CC(C)N2CCOCC2)cc1Cl ; *N1CCC(c2nc3cc(-c4c(C)noc4C)ccc3n2C2CCC(OC)CC2)N(c2ccc(F)c(F)c2)C1=O ; *N1CC(=O)N(c2ccc(F)c(F)c2)C(c2nc3cc(-c4c(C)noc4C)ccc3n2C2CCC(OC)CC2)C1 ; *OC1CCC(n2c(C3CCNC(=O)N3c3ccc(F)c(F)c3)nc3cc(-c4c(C)noc4C)ccc32)CC1] 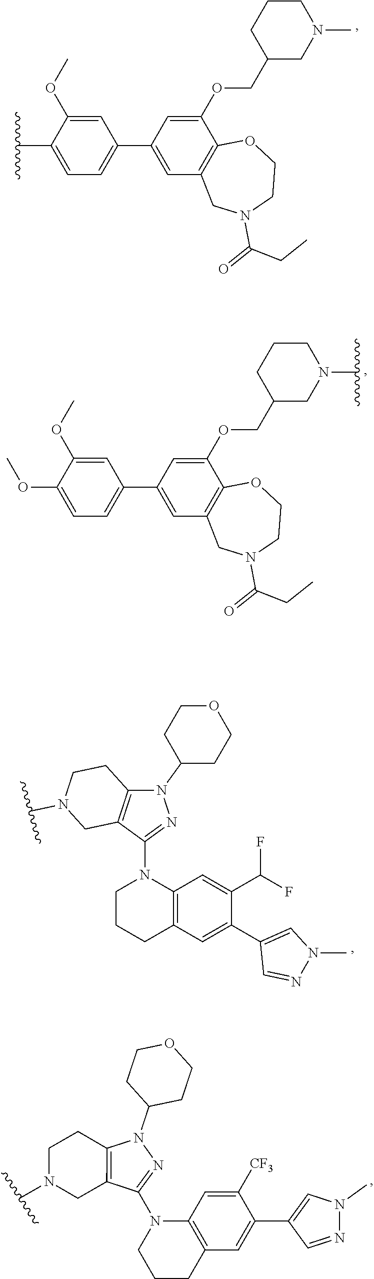 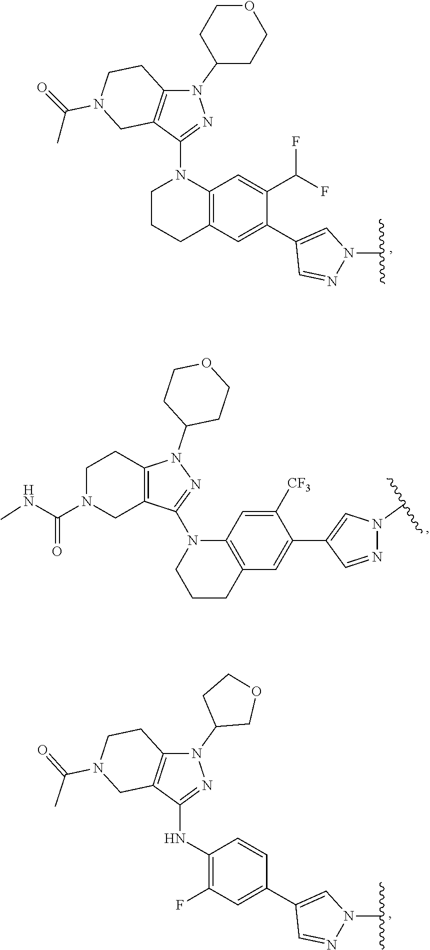 [SMILES: *n1cc(-c2cc3c(cc2C(F)F)N(c2nn(C4CCOCC4)c4c2CN(C(C)=O)CC4)CCC3)cn1 ; *n1cc(-c2cc3c(cc2C(F)(F)F)N(c2nn(C4CCOCC4)c4c2CN(C(=O)NC)CC4)CCC3)cn1 ; *n1cc(-c2ccc(Nc3nn(C4CCOC4)c4c3CN(C(C)=O)CC4)c(F)c2)cn1] 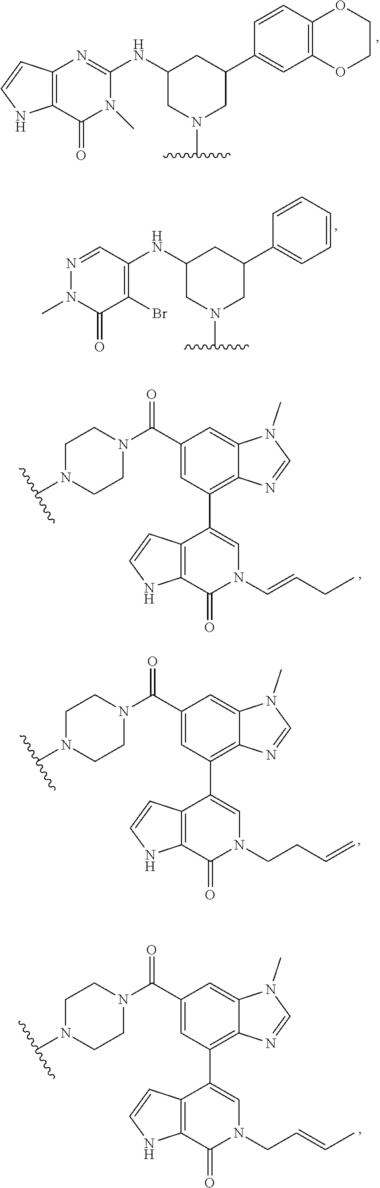 [SMILES: *N1CC(Nc2nc3cc[nH]c3c(=O)n2C)CC(c2ccc3c(c2)OCCO3)C1 ; *N1CC(Nc2cnn(C)c(=O)c2Br)CC(c2ccccc2)C1 ; *N1CCN(C(=O)c2cc(-c3cn(/C=C/CC)c(=O)c4[nH]ccc34)c3ncn(C)c3c2)CC1 ; *N1CCN(C(=O)c2cc(-c3cn(CCC=C)c(=O)c4[nH]ccc34)c3ncn(C)c3c2)CC1 ; *N1CCN(C(=O)c2cc(-c3cn(C/C=C/C)c(=O)c4[nH]ccc34)c3ncn(C)c3c2)CC1] 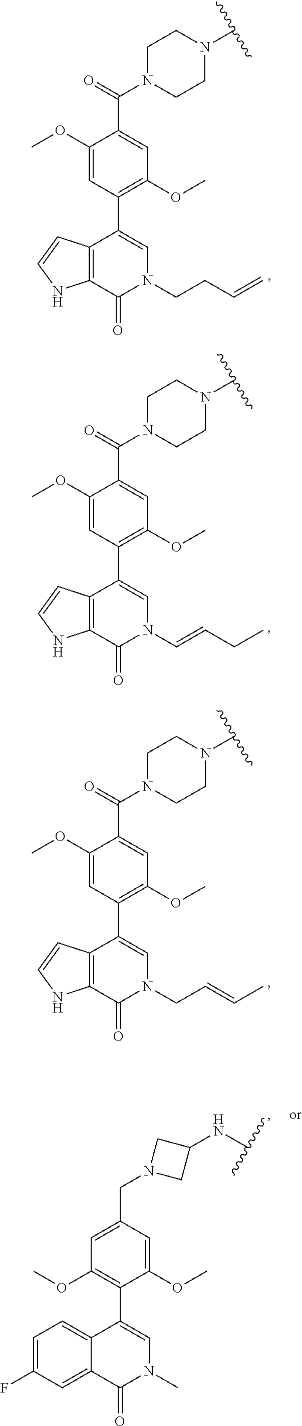 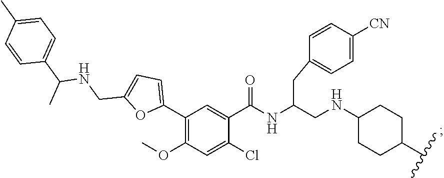 [SMILES: *C1CCC(NCC(Cc2ccc(C#N)cc2)NC(=O)c2cc(-c3ccc(CNC(C)c4ccc(C)cc4)o3)c(OC)cc2Cl)CC1]
or a pharmaceutically acceptable salt thereof.

38. A pharmaceutical composition comprising a transcription modulator molecule of any one of claims 1-37, or a pharmaceutically acceptable salt thereof, and a pharmaceutically acceptable excipient.

39. A method of modulation of the expression of fxn comprising contacting fxn with a transcription modulator molecule of any one of claims 1-37, or a pharmaceutically acceptable salt thereof.

40. A method of treatment of a disease or condition caused by expression of a defective fxn in a subject in need thereof, comprising administering to the subject an effective amount of a transcription modulator molecule of any one of claims 1-37, or a pharmaceutically acceptable salt thereof.

41. The method of claim 40, wherein the disease is Friedreich's ataxia (FA).

42. A method of treating Friedreich's ataxia (FA) in a subject in need thereof, comprising administering to the subject a transcription modulator molecule of any one of claims 1-37, or a pharmaceutically acceptable salt thereof.

43. The method of claim 42, wherein the method comprises alleviating one or more of muscular atrophy, ataxia, fasciculation, or dementia.
